(19) United States
(12) Patent Application Publication
(10) Pub. No.: US 2012/0220567 A1
(43) Pub. Date: Aug. 30, 2012

(54) BENZO-FUSED OXAZEPINE COMPOUNDS AS STEAROYL-COENZYME A DELTA-9 DESATURASE INHIBITORS

(76) Inventors: Gerald W. Shipps, JR., Stoneham, MA (US); Zhiwei Yang, San Diego, CA (US); Yongqi Deng, Newton, MA (US); Robert E. West, JR., Cranford, NJ (US); Dineshwar P. Sinha, Newton, NJ (US)

(21) Appl. No.: 13/386,533

(22) PCT Filed: Jul. 21, 2010
(86) PCT No.: PCT/US10/42734

§ 371 Date: Apr. 27, 2012

Related U.S. Application Data
(60) Provisional application No. 61/227,943, filed on Jul. 23, 2009.

(51) Int. Cl. A61K 31/553; A61P 3/10; A61P 5/48; A61P 3/00; A61P 7/00; A61P 1/16; A61P 17/00; A61P 3/06; A61P 9/10; C07D 498/10; A61P 3/04

(52) U.S. Cl. 514/211.09; 540/543

(57) ABSTRACT

The present invention relates to certain 4,5-dihydro-3H-spiro[benzo[b]-[1,4]oxazepine compounds of the Formula (I) or a pharmaceutically acceptable salt thereof, wherein R¹, R², R³, R⁴, R⁵, m, n, o, and p are as herein described. In addition, the invention relates to compositions comprising at least one such compound, and methods of using the compounds for treating or preventing disorders such as non-insulin dependent (Type 2) diabetes, insulin resistance, hyperglycemia, a lipid disorder, obesity, fatty liver disease, or a skin disorder.

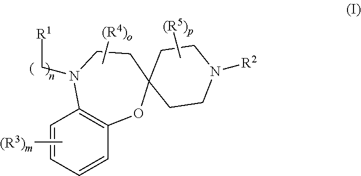

### FIELD OF THE INVENTION

The present invention relates to certain 4,5-dihydro-3H-spiro[benzo[b]-[1,4]oxazepine compounds of the Formula (I) (also referred to as the “Benzo-Fused Oxazepine Compounds”), compositions comprising at least one Benzo-Fused Oxazepine Compound, and methods of using Benzo-Fused Oxazepine Compounds for treating or preventing disorders such as non-insulin dependent (Type 2) diabetes, insulin resistance, hyperglycemia, a lipid disorder, obesity, fatty liver disease, or a skin disorder.

### BACKGROUND OF THE INVENTION

Stearoyl-coenzyme A (CoA) desaturase (SCD) is involved in the de novo synthesis of monounsaturated fats from saturated fatty acids (see e.g., Ntambi (1999) J. Lipid Res. 40, 1549 for a review). The major products of SCD are palmitoyl-CoA and oleoyl-CoA, which are formed by desaturation of palmitoyl-CoA and stearoyl-CoA, respectively. Oleate is found to be the major monounsaturated fatty acid of membrane phospholipids, triglycerides, cholesterol esters, wax esters and alkyl-1,2-diacylglycerol. The ratio of saturated to unsaturated fatty acids is one of the factors influencing membrane fluidity and its alteration is important in diseases like aging, cancer, diabetes, obesity, and neurological, vascular and heart diseases (Biochem. Biophys. Acta., 431, 469-480 (1976); J. Biol. Chem., 268, 6823-6826 (1993); Diabetes, 40, 280-289 (1991); Neurochem Res., 26, 771-782 (1994); Arthritis Rheum., 43, 894-900 (2000); Cancer Lett., 173, 139-144 (2001)).

Depending on the species, highly homologous isoforms of SCD exist differing primarily in tissue distribution. For instance, in mice, four SCD isoforms have been identified, while two SCD isoforms have been found in humans, SCD1 and SCD5. In humans, adipose and liver tissue show highest expression of SCD1, while brain and pancreatic tissues show highest expression of SCD5. Flowers and Ntambi (2008) Curr. Opin. Lipidol. 19, 248.

In vivo studies in mice support the central role of SCD in both fatty acid metabolism and metabolic conditions. Mice strains with a naturally occurring mutation in one of the isoforms of SCD, SCD1, and mice which have a targeted disruption in the SCD1 gene show reduced fatty acid and triglyceride synthesis in response to a high carbohydrate diet as compared to the amounts in wild type mice. Furthermore, mice which have a targeted disruption in the SCD1 gene show reduced body adiposity, increased insulin sensitivity and resistance to diet-induced obesity. Ntambi and Miyazaki (2003) Curr. Opin. Lipidol. 14, 255. Mice which were injected intraperitoneally with SCD-1 targeted antisense oligonucleotide showed improved insulin sensitivity and prevented occurrence of obesity in the mice in response to high fat diets. In view of the experimental evidence described above, modulation of SCD represents a promising therapeutic strategy for the treatment of obesity and related metabolic disorders.

In addition to the above-described findings, studies in mice further suggest that SCD1 activity is important to maintaining the normal functioning of the skin and eyelid as a result of its major role in lipid synthesis within sebaceous and meibomian glands. Both mice carrying a naturally occurring mutation in the SCD1 gene (Zheng et al. (1999) Nature Genet. 23, 268) and mice which have a targeted disruption in the SCD1 gene (Miyazaki et al. (2001) J. Nutr. 131, 2260) develop skin and eye abnormalities. These changes include hair loss as well as atrophy of the sebaceous and meibomian glands.

In humans, sebaceous glands secrete an oily substance called sebum which is distributed onto the skin surface which decreases the skin's stratum corneum layer's permeability and prevents the skin from cracking. These glands are present in all areas of the skin except for the palms of the hands and soles of the feet. The highest concentration of sebaceous glands occurs on the scalp and face. Despite the important functions that sebum plays, many individuals experience excess sebum production which condition is associated with increased incidence of dermatological conditions such as acne or seborrheic dermatitis. Even in individuals without acne, excess sebum production detracts from the cosmetic appearance of the skin and hair by causing the skin to look shiny, greasy or oily and hair to look limp and dirty. Decreasing the production of sebum will alleviate oily skin and hair in individuals experiencing these conditions.

In view of the findings described above, there is a need for identifying molecules that modulate SCD activity and are useful for the treatment of metabolic disorders, such as obesity and type 2 diabetes, and skin disorders such as acne.

### SUMMARY OF THE INVENTION

In one aspect, the present invention provides Compounds of Formula (I) (herein referred to as the “Benzo-Fused Oxazepine Compounds”):

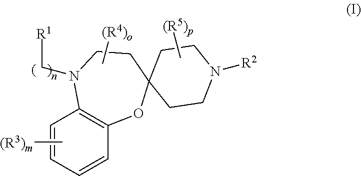

or a pharmaceutically acceptable salt thereof, wherein

R¹ is aryl or heteroaryl, wherein said aryl or heteroaryl of R¹ is unsubstituted or substituted with one to four moieties, wherein said moieties are the same or different, and wherein said moieties are selected from the group consisting of alkyl, alkoxy, haloalkyl, halo, —CN, —C(O)—R⁶, —OCF₃, and R⁷;

R² is —C(O)—Y, wherein Y is alkyl or cycloalkyl,

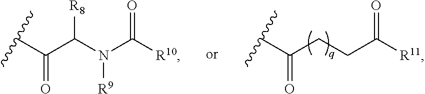

or R² is aryl or heteroaryl, wherein said aryl or heteroaryl is unsubstituted or substituted with one to three moieties, which moieties are the same or different, and wherein said moieties are selected from Z, wherein Z is Z¹ or Z²;

Z¹ is alkyl, alkoxy, halo, haloalkyl, —CN, —C(O)—OH, —C(O)—O-alkyl, —C(O)-β-cycloalkyl, —C(O)—N(R¹²)₂, —OCF₃, aryl, heteroaryl, aryl substituted with alkyl, and heteroaryl substituted by alkyl;

Z² is

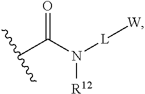

wherein L is a direct bond such that W is bonded directly to the illustrated N atom of —N(R¹²)—, or L is —(CH₂)ₓ—, —CH₂—C(H)(OH)—, or —CH₂—C(H)(OH)—CH₂—;

W is —C(O)OR¹³, —C(O)NR¹², —S(O)alkyl, —S(O)₂alkyl, —CF₃, —C(H)(OH)—CH₂OH, —CH₂OH, —C(H)(CH₃)OH, cycloalkyl, aryl, heteroaryl, heterocyclyl, wherein said cycloalkyl, aryl, heteroaryl, or heterocyclyl of W is unsubstituted or substituted with one to three moieties, wherein said moieties are selected from the group consisting of alkyl, hydroxyl, alkoxy, halo, —CF₃, —OCF₃, or —CN;

with the proviso that when W is —C(O)OR¹³, —C(O)NR¹², —S(O)alkyl, —S(O)₂alkyl, —CF₃, —C(H)(OH)—CH₂OH, or —CH₂OH or, then L must be —(CH₂)ₓ—, —CH₂—C(H)(OH)—, or —CH₂—C(H)(OH)—CF₁₂—;

each R³ is independently alkyl, alkoxy, or halo;

each R⁴ is independently alkyl or halo;

each R⁵ is independently alkyl or halo;

R⁶ is —OH, —O-alkyl, —O-cycloalkyl, —N(R¹²)₂,

R⁷ is —O-aryl, —O-heteroaryl, —N(R¹²)-aryl, or —N(R¹²)-heteroaryl,

R⁸ is H, alkyl, or hydroxyalkyl;

R⁹ is H or alkyl;

R¹⁰ is alkyl, cycloalkyl, aryl, heteroaryl, or heterocyclyl;

R¹¹ is OH, alkyl, or cycloalkyl;

each R¹² is independently H, alkyl, alkoxyethyl, alkoxypropyl, dialkylaminoethyl, dialkylaminopropyl, or wherein two R¹² are geminally substituted on a N atom, the two R¹² together with the N atom on which they are substituted form a 4- to 8-membered heterocyclyl;

R¹³ is H, alkyl, or cycloalkyl;

m is 0, 1, 2, 3, or 4;

n is 1, 2, or 3;

o is 0 or 1;

p is 0, 1, 2, 3, or 4;

q is 1 or 2; and

x is 1, 2, 3, 4, or 5;

with the proviso that the compound is other than
N-(2-oxo-2-(5-(4-phenoxybenzyl)-4,5-dihydro-3H-spiro[benzo[b][1,4]oxazepine-2,4′-piperidine]-1′-yl)ethyl)acetamide;
N-(2-(5-(4-isopropoxybenzyl)-4,5-dihydro-3H-spiro[benzo[b][1,4]oxazepine-2,4′-piperidine]-1′-yl)-2-oxoethyl)acetamide;
N-(2-(5-(biphenyl-4-ylmethyl)-4,5-dihydro-3H-spiro[benzo[b][1,4]oxazepine-2,4′-piperidine]-1′-yl)-2-oxoethyl)acetamide;
N-(2-(5-(4-tert-butylbenzyl)-4,5-dihydro-3H-spiro[benzo[b][1,4]oxazepine-2,4′-piperidine]-1′-yl)-2-oxoethyl)acetamide;
N-(2-(5-(4-isopropylbenzyl)-4,5-dihydro-3H-spiro[benzo[b][1,4]oxazepine-2,4′-piperidine]-1′-yl)-2-oxoethyl)acetamide;
4-(5-(4-isopropylbenzyl)-4,5-dihydro-3H-spiro[benzo[b][1,4]oxazepine-2,4′-piperidine]-1-yl)-4-oxobutanamide;
N-(2-(5-(4-(4-fluorobenzyloxy)benzyl)-4,5-dihydro-3H-spiro[benzo[b][1,4]oxazepine-2,4′-piperidine]-1′-yl)-2-oxoethyl)acetamide;
4-(5-(4-tert-butylbenzyl)-4,5-dihydro-3H-spiro[benzo[b][1,4]oxazepine-2,4′-piperidine]-1′-yl)-4-oxobutanamide;
4-(5-(biphenyl-4-ylmethyl)-4,5-dihydro-3H-spiro[benzo[b][1,4]oxazepine-2,4′-piperidine]-1′-yl)-4-oxobutanamide;
5-(4-ethylbenzyl)-N-(4-(methoxymethyl)phenyl)-4,5-dihydro-3H-spiro[benzo[b][1,4]oxazepine-2,4′-piperidine]-1′-carboxamide;
2-(benzo[c][1,2,5]thiadiazol-4-yl)-1-(5-(4-isopropylbenzyl)-4,5-dihydro-3 H-spiro[benzo[b][1,4]oxazepine-2,4′-piperidine]-1′-yl)ethanone;
N-(2-(5-(benzofuran-2-ylmethyl)-4,5-dihydro-3H-spiro[benzo[b][1,4]oxazepine-2,4′-piperidine]-1′-yl)-2-oxoethyl)furan-2-carboxamide;
N-(2-oxo-2-(5-(3-(trifluoromethyl)benzyl)-4,5-dihydro-3H-spiro[benzo[b][1,4]oxazepine-2,4′-piperidine]-1′-yl)ethyl)furan-2-carboxamide;
4-(5-(4-isopropoxybenzyl)-4,5-dihydro-3H-spiro[benzo[b][1,4]oxazepine-2,4′-piperidine]-1′-yl)-4-oxobutanamide;
N-(2-(5-(2-ethylbenzyl)-4,5-dihydro-3H-spiro[benzo[b][1,4]oxazepine-2,4′-piperidine]-1′-yl)-2-oxoethyl)acetamide;
N-(benzo[c][1,2,5]thiadiazol-4-yl)-5-(biphenyl-4-ylmethyl)-4,5-dihydro-3H-spiro[benzo[b][1,4]oxazepine-2,4′-piperidine]-1-carboxamide;
3-(5-(4-tert-butylbenzyl)-4,5-dihydro-3H-spiro[benzo[b][1,4]oxazepine-2,4′-piperidine]-1′-yl)-3-oxopropanenitrile;
N-(benzo[c][1,2,5]thiadiazol-4-yl)-5-(4-tert-butylbenzyl)-4,5-dihydro-3H-spiro[benzo[b][1,4]oxazepine-2,4′-piperidine]-1′-carboxamide;
methyl 4-((1′-(2-acetamidoacetyl)-3H-spiro[benzo[b][1,4]oxazepine-2,4′-piperidine]-5(4H)-yl)methyl)benzoate;
N-(2-(5-(2,3-dimethylbenzyl)-4,5-dihydro-3H-spiro[benzo[b][1,4]oxazepine-2,4′-piperidine]-1′-yl)-2-oxoethyl)acetamide;
4-oxo-4-(5-(4-phenoxybenzyl)-4,5-dihydro-3H-spiro[benzo[b][1,4]oxazepine-2,4′-piperidine]-1′-yl)butanamide;
N-(2-(5-((5-(3-chlorophenyl)furan-2-yl)methyl)-4,5-dihydro-3H-spiro[benzo[b][1,4]oxazepine-2,4′-piperidine]-1′-yl)-2-oxoethyl)furan-2-carboxamide;
N-(2-oxo-2-(5-(4-(pyridin-2-yl)benzyl)-4,5-dihydro-3H-spiro[benzo[b][1,4]oxazepine-2,4′-piperidine]-1′-yl)ethyl)acetamide;
N-(2-(5-(3-fluoro-2-methylbenzyl)-4,5-dihydro-3H-spiro[benzo[b][1,4]oxazepine-2,4′-piperidine]-1′-yl)-2-oxoethyl)acetamide;
N-(benzo[c][1,2,5]thiadiazol-4-yl)-5-(4-phenoxybenzyl)-4,5-dihydro-3H-spiro[benzo[b][1,4]oxazepine-2,4′-piperidine]-1′-carboxamide;
N-(2-(5-(2,5-difluorobenzyl)-4,5-dihydro-3H-spiro[benzo[b][1,4]oxazepine-2,4′-piperidine]-1′-yl)-2-oxoethyl)acetamide;
N-(3,5-difluorophenyl)-5-(4-isopropylbenzyl)-4,5-dihydro-3H-spiro[benzo[b][1,4]oxazepine-2,4′-piperidine]-1′-carboxamide;
3-oxo-3-(5-(3-(trifluoromethyl)benzyl)-4,5-dihydro-3H-spiro[benzo[b][1,4]oxazepine-2,4′-piperidine]-1′-yl)propanenitrile;
N-(2-(5-(4-fluoro-3-methoxybenzyl)-4,5-dihydro-3H-spiro[benzo[b][1,4]oxazepine-2,4′-piperidine]-1′-yl)-2-oxoethyl)acetamide;
5-(2-oxo-2-(5-(3-(trifluoromethyl)benzyl)-4,5-dihydro-3H-spiro[benzo[b][1,4]oxazepine-2,4′-piperidine]-1′-yl)ethyl)imidazolidine-2,4-dione;
N-(2-(5-(4-ethyl benzyl)-4,5-dihydro-3H-spiro[benzo[b][1,4]oxazepine-2,4′-piperidine]-1′-yl)-2-oxoethyl)benzamide;
N-(2-oxo-2-(5-(3-phenylpropyl)-4,5-dihydro-3H-spiro[benzo[b][1,4]oxazepine-2,4′-piperidine]-1′-yl)ethyl)acetamide;
N-(2-(5-(3-methoxybenzyl)-4,5-dihydro-3H-spiro[benzo[b][1,4]oxazepine-2,4′-piperidine]-1′-yl)-2-oxoethyl)acetamide;
N-(2-(5-(2-ethoxybenzyl)-4,5-dihydro-3H-spiro[benzo[b][1,4]oxazepine-2,4′-piperidine]-1′-yl)-2-oxoethyl)acetamide;
4-oxo-4-(5-(3-(trifluoromethyl)benzyl)-4,5-dihydro-3H-spiro[benzo[b][1,4]oxazepine-2,4′-piperidine]-1′-yl)butanamide;
N-(2-oxo-2-(5-(3-(trifluoromethyl)benzyl)-4,5-d i hydro-3H-spiro[benzo[b][1,4]oxazepine-2,4′-piperidine]-1′-yl)ethyl)benzamide;
4-(5-((5-(3-chlorophenyl)furan-2-yl)methyl)-4,5-dihydro-3H-spiro[benzo[b][1,4]oxazepine-2,4′-piperidine]-1′-yl)-4-oxobutanamide;
4-(5-(2-ethyl benzyl)-4,5-dihydro-3H-spiro[benzo[b][1,4]oxazepine-2,4′-piperidine]-1′-yl)-4-oxobutanamide;
N-(2-oxo-2-(5-(thiophen-2-ylmethyl)-4,5-dihydro-3H-spiro[benzo[b][1,4]oxazepine-2,4′-piperidine]-1′-yl)ethyl)acetamide;
3-(5-(biphenyl-4-ylmethyl)-4,5-dihydro-3H-spiro[benzo[b][1,4]oxazepine-2,4′-piperidine]-1′-yl)-3-oxopropanenitrile;
3-(5-(4-isopropylbenzyl)-4,5-dihydro-3H-spiro[benzo[b][1,4]oxazepine-2,4′-piperidine]-1′-yl)-3-oxopropanenitrile;
5-(4-ethylbenzyl)-N-(4-fluorophenyl)-4,5-dihydro-3H-spiro[benzo[b][1,4]oxazepine-2,4′-piperidine]-1′-carboxamide; (2S,3S)-methyl 2-(5-(4-ethylbenzyl)-4,5-dihydro-3H-spiro[benzo[b][1,4]oxazepine-2,4′-piperidine]-1′-ylcarboxamido)-3-methylpentanoate;
N-(2-(5-(4-ethylbenzyl)-4,5-dihydro-3H-spiro[benzo[b][1,4]oxazepine-2,4′-piperidine]-1-yl)-2-oxoethyl)acetamide; and
N-(2-oxo-2-(5-(3-(trifluoromethyl)benzyl)-4,5-dihydro-3H-spiro[benzo[b][1,4]oxazepine-2,4′-piperidine]-1-yl)ethyl)acetamide.

In one embodiment of the Compound of Formula (I), R² is heteroaryl, wherein said heteroaryl is unsubstituted or substituted with one to three moieties, which moieties are the same or different, wherein said moieties are selected from Z, wherein Z is as described above.

The invention also provides a method for treating a disorder selected from non-insulin dependent (Type 2) diabetes, insulin resistance, hyperglycemia, a lipid disorder, obesity, fatty liver disease, or a skin disorder comprising administering a Benzo-Fused Oxazepine Compound, or a pharmaceutically acceptable salt thereof, to a patient, ag., a human patient, in need of such treatment. For instance, in some embodiments, the disorder is a lipid disorder, which is dyslipidemia, hyperlipidemia, atherosclerosis, hypercholesterolemia, low LDL, or high LDL. In other embodiments, the disorder being treated is a skin disorder.

The invention also provides a use of a Benzo-Fused Oxazepine Compound for treating a disorder selected from non-insulin dependent (Type 2) diabetes, insulin resistance, hyperglycemia, a lipid disorder, obesity, fatty liver disease, or a skin disorder. For instance, in some embodiments, the disorder is a lipid disorder, which is dyslipidemia, hyperlipidemia, atherosclerosis, hypercholesterolemia, low LDL, or high LDL. In other embodiments, the disorder is a skin disorder.

The present invention further provides pharmaceutical compositions comprising an effective amount of at least one Benzo-Fused Oxazepine Compound or a pharmaceutically acceptable salt thereof, and a pharmaceutically acceptable carrier. In some embodiments, the pharmaceutical compositions can be useful for treating non-insulin dependent (Type 2) diabetes, insulin resistance, hyperglycemia, a lipid disorder, obesity, fatty liver disease, or a skin disorder in a subject in need of such treatment.

The details of the invention are set forth in the accompanying detailed description below.

Although any methods and materials similar to those described herein can be used in the practice or testing of the present invention, illustrative methods and materials are now described. Other features, objects, and advantages of the invention will be apparent from the description and the claims. All patents and publications cited in this specification are incorporated herein by reference.

### DETAILED DESCRIPTION OF THE INVENTION

The present invention provides Benzo-Fused Oxazepine Compounds, pharmaceutical compositions comprising at least one Benzo-Fused Oxazepine Compound, and methods of using the Benzo-Fused Oxazepine Compounds for treating a metabolic disorder or skin disease in a patient, e.g., a human patient.

### Definitions and Abbreviations

As used above, and throughout this disclosure, the following terms, unless otherwise indicated, shall be understood to have the following meanings:

“Patient” includes both human and animals.

“Mammal” means humans and other mammalian animals.

“Alkyl” means an aliphatic hydrocarbon group which may be straight or branched and comprising about 1 to about 20 carbon atoms in the chain.

Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. “Alkyl” may be unsubstituted or optionally substituted by one or more substituents which may be the same or different, each substituent being independently selected from the group consisting of halo, alkyl, aryl, cycloalkyl, cyano, hydroxy, alkoxy, alkylthio, amino, oxime (e.g., ═N—OH), —NH(alkyl), —NH(cycloalkyl), —N(alkyl)₂, —O—C(O)-alkyl, —O—C(O)-aryl, —O—C(O)-cycloalkyl, —SF₅, carboxy and —C(O)O-alkyl. Non-limiting examples of suitable alkyl groups include methyl, ethyl, n-propyl, isopropyl and t-butyl.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. “Alkenyl” may be unsubstituted or optionally substituted by one or more substituents which may be the same or different, each substituent being independently selected from the group consisting of halo, alkyl. aryl, cycloalkyl, cyano, alkoxy and —S(alkyl). Non-limiting examples of suitable alkenyl groups include ethenyl, propenyl, n-butenyl, 3-methylbut-2-enyl, n-pentenyl, octenyl and decenyl.

“Alkylene” means a difunctional group obtained by removal of a hydrogen atom from an alkyl group that is defined above. Non-limiting examples of alkylene include methylene, ethylene and propylene.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl and 3-methylbutynyl. “Alkynyl” may be unsubstituted or optionally substituted by one or more substituents which may be the same or different, each substituent being independently selected from the group consisting of alkyl, aryl and cycloalkyl.

“Aryl” means an aromatic monocyclic or multicyclic ring system comprising about 6 to about 14 carbon atoms, preferably about 6 to about 10 carbon atoms. The aryl group can be optionally substituted with one or more “ring system substituents” which may be the same or different, and are as defined herein. Non-limiting examples of suitable aryl groups include phenyl and naphthyl.

“Heteroaryl” means an aromatic monocyclic or multicyclic ring system comprising about 5 to about 14 ring atoms, preferably about 5 to about 10 ring atoms, in which one or more of the ring atoms is an element other than carbon, for example nitrogen, oxygen or sulfur, alone or in combination. Preferred heteroaryls contain about 5 to about 6 ring atoms. The “heteroaryl” can be optionally substituted by one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be optionally oxidized to the corresponding N-oxide. “Heteroaryl” may also include a heteroaryl as defined above fused to an aryl as defined above. Non-limiting examples of suitable heteroaryls include pyridyl, pyrazinyl, furanyl, thienyl, pyrimidinyl, pyridone (including N-substituted pyridones), isoxazolyl, isothiazolyl, oxazolyl, thiazolyl, pyrazolyl, furazanyl, pyrrolyl, pyrazolyl, triazolyl, 1,2,4-thiadiazolyl, pyrazinyl, pyridazinyl, quinoxalinyl, phthalazinyl, oxindolyl, imidazo[1,2-a]pyridinyl, imidazo[2,1-b]thiazolyl, benzofurazanyl, indolyl, azaindolyl, benzimidazolyl, benzothienyl, quinolinyl, imidazolyl, thienopyridyl, quinazolinyl, thienopyrimidyl, pyrrolopyridyl, imidazopyridyl, isoquinolinyl, benzoazaindolyl, 1,2,4-triazinyl, benzothiazolyl and the like. The term “heteroaryl” also refers to partially saturated heteroaryl moieties such as, for example, tetrahydroisoquinolyl, tetrahydroquinolyl and the like.

“Aralkyl” or “arylalkyl” means an aryl-alkyl-group in which the aryl and alkyl are as previously described. Preferred aralkyls comprise a lower alkyl group. Non-limiting examples of suitable aralkyl groups include benzyl, 2-phenethyl and naphthalenylmethyl. The bond to the parent moiety is through the alkyl.

“Alkylaryl” means an alkyl-aryl-group in which the alkyl and aryl are as previously described. Preferred alkylaryls comprise a lower alkyl group. Non-limiting example of a suitable alkylaryl group is tolyl. The bond to the parent moiety is through the aryl.

“Cycloalkyl” means a non-aromatic mono- or multicyclic ring system comprising about 3 to about 10 carbon atoms, preferably about 5 to about 10 carbon atoms. Preferred cycloalkyl rings contain about 5 to about 7 ring atoms. The cycloalkyl can be optionally substituted with one or more “ring system substituents” which may be the same or different, and are as defined above. Non-limiting examples of suitable monocyclic cycloalkyls include cyclopropyl, cyclopentyl, cyclohexyl, cycloheptyl and the like. Non-limiting examples of suitable multicyclic cycloalkyls include 1-decalinyl, norbornyl, adamantyl and the like.

“Cycloalkylalkyl” means a cycloalkyl moiety as defined above linked via an alkyl moiety (defined above) to a parent core. Non-limiting examples of suitable cycloalkylalkyls include cyclohexylmethyl, adamantylmethyl and the like.

“Cycloalkenyl” means a non-aromatic mono or multicyclic ring system comprising about 3 to about 10 carbon atoms, preferably about 5 to about 10 carbon atoms which contains at least one carbon-carbon double bond. Preferred cycloalkenyl rings contain about 5 to about 7 ring atoms. The cycloalkenyl can be optionally substituted with one or more “ring system substituents” which may be the same or different, and are as defined above. Non-limiting examples of suitable monocyclic cycloalkenyls include cyclopentenyl, cyclohexenyl, cyclohepta-1,3-dienyl, and the like. A non-limiting example of a suitable multicyclic cycloalkenyl is norbornylenyl.

“Cycloalkenylalkyl” means a cycloalkenyl moiety as defined above linked via an alkyl moiety (defined above) to a parent core. Non-limiting examples of suitable cycloalkenylalkyls include cyclopentenylmethyl, cyclohexenylmethyl and the like.

“Halo” means fluoro, chloro, bromo, or iodo. Preferred halos are fluoro, chloro and bromo.

“Ring system substituent” means a substituent attached to an aromatic or non-aromatic ring system which, for example, replaces an available hydrogen on the ring system. Ring system substituents may be the same or different, each being independently selected from the group consisting of alkyl, alkenyl, alkynyl, aryl, heteroaryl, aralkyl, alkylaryl, heteroaralkyl, heteroarylalkenyl, heteroarylalkynyl, alkylheteroaryl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylthio, arylthio, heteroarylthio, aralkylthio, heteroaralkylthio, cycloalkyl, heterocyclyl, —SF₅, —OSF₅ (for aryl), —O—C(O)-alkyl, —O—C(O)-aryl, —O—C(O)-cycloalkyl, —C(═N—CN)—NH₂, —C(═NH)—NH₂, —C(═NH)—NH(alkyl), oxime (e.g., ═N—OH), —NY₁Y₂, -alkyl-NY₁Y₂, —C(O)NY₁Y₂, —SO₂NY₁Y₂ and —SO₂NY₁Y₂, wherein Y₁ and Y₂ can be the same or different and are independently selected from the group consisting of hydrogen, alkyl, aryl, cycloalkyl, and aralkyl. “Ring system substituent” may also mean a single moiety which simultaneously replaces two available hydrogens on two adjacent carbon atoms (one H on each carbon) on a ring system. Examples of such a moiety are methylene dioxy, ethylenedioxy, —C(CH₃)₂— and the like which form moieties such as, for example:

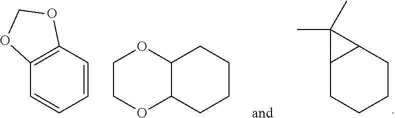

“Heteroarylalkyl” means a heteroaryl moiety as defined above linked via an alkyl moiety (defined above) to a parent core. Non-limiting examples of suitable heteroaryls include 2-pyridinylmethyl, quinolinylmethyl and the like.

“Heterocyclyl” means a non-aromatic saturated monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur, alone or in combination. There are no adjacent oxygen and/or sulfur atoms present in the ring system. Preferred heterocyclyls contain about 5 to about 6 ring atoms. The prefix aza, oxa or thia before the heterocyclyl root name means that at least a nitrogen, oxygen or sulfur atom respectively is present as a ring atom. Any —NH in a heterocyclyl ring may exist protected such as, for example, as an —N(Boc), —N(CBz), —N(Tos) group and the like; such protections are also considered part of this invention. The heterocyclyl can be optionally substituted by one or more “ring system substituents” which may be the same or different, and are as defined herein. The nitrogen or sulfur atom of the heterocyclyl can be optionally oxidized to the corresponding N-oxide, S-oxide or S,S-dioxide. Non-limiting examples of suitable monocyclic heterocyclyl rings include piperidyl, pyrrolidinyl, piperazinyl, morpholinyl, thiomorpholinyl, thiazolidinyl, 1,4-dioxanyl, tetrahydrofuranyl, tetrahydrothiophenyl, lactam, lactone, and the like. “Heterocyclyl” also includes heterocyclyl rings as described above wherein ═O replaces two available hydrogens on the same ring carbon atom. An example of such a moiety is pyrrolidone:

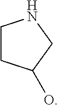

“Heterocyclylalkyl” means a heterocyclyl moiety as defined above linked via an alkyl moiety (defined above) to a parent core. Non-limiting examples of suitable heterocyclylalkyls include piperidinylmethyl, piperazinylmethyl and the like.

“Heterocyclenyl” means a non-aromatic monocyclic or multicyclic ring system comprising about 3 to about 10 ring atoms, preferably about 5 to about 10 ring atoms, in which one or more of the atoms in the ring system is an element other than carbon, for example nitrogen, oxygen or sulfur atom, alone or in combination, and which contains at least one carbon-carbon double bond or carbon-nitrogen double bond. There are no adjacent oxygen and/or sulfur atoms present in the ring system. Preferred heterocyclenyl rings contain about 5 to about 6 ring atoms. The prefix aza, oxa or thia before the heterocyclenyl root name means that at least a nitrogen, oxygen or sulfur atom respectively is present as a ring atom. The heterocyclenyl can be optionally substituted by one or more ring system substituents, wherein “ring system substituent” is as defined above. The nitrogen or sulfur atom of the heterocyclenyl can be optionally oxidized to the corresponding N-oxide. S-oxide or S,S-dioxide. Non-limiting examples of suitable heterocyclenyl groups include 1,2,3,4-tetrahydropyridinyl, 1,2-dihydropyridinyl, 1,4-dihydropyridinyl, 1,2,3,6-tetrahydropyridinyl, 1,4,5,6-tetrahydropyrimidinyl, 2-pyrrolinyl, 3-pyrrolinyl, 2-imidazolinyl, 2-pyrazolinyl, dihydroimidazolyl, dihydrooxazolyl, dihydrooxadiazolyl, dihydrothiazolyl, 3,4-dihydro-2H-pyranyl, dihydrofuranyl, fluorodihydrofuranyl, 7-oxabicyclo[2.2.1]heptenyl, dihydrothiophenyl, dihydrothiopyranyl, and the like. “Heterocyclenyl” also includes heterocyclenyl rings as described above wherein ═O replaces two available hydrogens on the same ring carbon atom. An example of such a moiety is pyrrolidinone:

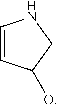

“Heterocyclenylalkyl” means a heterocyclenyl moiety as defined above linked via an alkyl moiety (defined above) to a parent core.

It should be noted that in hetero-atom containing ring systems of this invention, there are no hydroxyl groups on carbon atoms adjacent to a N, O or S, as well as there are no N or S groups on carbon adjacent to another heteroatom. Thus, for example, in the ring:

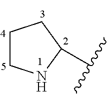

there is no —OH attached directly to carbons marked 2 and 5.

It should also be noted that tautomeric forms such as, for example, the moieties:

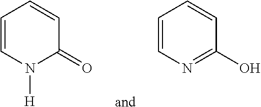

are considered equivalent in certain embodiments of this invention.

“Alkynylalkyl” means an alkynyl-alkyl-group in which the alkynyl and alkyl are as previously described. Preferred alkynylalkyls contain a lower alkynyl and a lower alkyl group. The bond to the parent moiety is through the alkyl. Non-limiting examples of suitable alkynylalkyl groups include propargylmethyl.

“Heteroaralkyl” means a heteroaryl-alkyl-group in which the heteroaryl and alkyl are as previously described. Preferred heteroaralkyls contain a lower alkyl group. Non-limiting examples of suitable aralkyl groups include pyridylmethyl, and quinolin-3-ylmethyl. The bond to the parent moiety is through the alkyl.

“Hydroxyalkyl” means a HO-alkyl-group in which alkyl is as previously defined. Preferred hydroxyalkyls contain lower alkyl. Non-limiting examples of suitable hydroxyalkyl groups include hydroxymethyl and 2-hydroxyethyl.

“Acyl” means an H—C(O)—, alkyl-C(O)— or cycloalkyl-C(O)—, group in which the various groups are as previously described. The bond to the parent moiety is through the carbonyl. Preferred acyls contain a lower alkyl. Non-limiting examples of suitable acyl groups include formyl, acetyl and propanoyl.

“Aroyl” means an aryl-C(O)— group in which the aryl group is as previously described. The bond to the parent moiety is through the carbonyl. Non-limiting examples of suitable groups include benzoyl and 1-naphthoyl.

“Alkoxy” means an alkyl-O— group in which the alkyl group is as previously described. Non-limiting examples of suitable alkoxy groups include methoxy, ethoxy, n-propoxy, isopropoxy and n-butoxy. The bond to the parent moiety is through the ether oxygen.

“Aryloxy” means an aryl-O— group in which the aryl group is as previously described. Non-limiting examples of suitable aryloxy groups include phenoxy and naphthoxy. The bond to the parent moiety is through the ether oxygen.

“Aralkyloxy” means an aralkyl-O— group in which the aralkyl group is as previously described. Non-limiting examples of suitable aralkyloxy groups include benzyloxy and 1- or 2-naphthalenemethoxy. The bond to the parent moiety is through the ether oxygen.

“Alkylthio” means an alkyl-S— group in which the alkyl group is as previously described. Non-limiting examples of suitable alkylthio groups include methylthio and ethylthio. The bond to the parent moiety is through the sulfur.

“Arylthio” means an aryl-S— group in which the aryl group is as previously described. Non-limiting examples of suitable arylthio groups include phenylthio and naphthylthio. The bond to the parent moiety is through the sulfur.

“Aralkylthio” means an aralkyl-S— group in which the aralkyl group is as previously described. Non-limiting example of a suitable aralkylthio group is benzylthio. The bond to the parent moiety is through the sulfur.

“Alkoxycarbonyl” means an alkyl-O—C(O)— group. Non-limiting examples of suitable alkoxycarbonyl groups include methoxycarbonyl and ethoxycarbonyl. The bond to the parent moiety is through the carbonyl.

“Aryloxycarbonyl” means an aryl-O—C(O)— group. Non-limiting examples of suitable aryloxycarbonyl groups include phenoxycarbonyl and naphthoxycarbonyl. The bond to the parent moiety is through the carbonyl.

“Aralkoxycarbonyl” means an aralkyl-O—C(O)— group. Non-limiting example of a suitable aralkoxycarbonyl group is benzyloxycarbonyl. The bond to the parent moiety is through the carbonyl.

“Alkylsulfonyl” means an alkyl-S(O₂)— group. Preferred groups are those in which the alkyl group is lower alkyl. The bond to the parent moiety is through the sulfonyl.

“Arylsulfonyl” means an aryl-S(O₂)— group. The bond to the parent moiety is through the sulfonyl.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. Reference to a “stable compound” or “stable structure” means a compound that is sufficiently robust to survive isolation to a useful degree of purity from a reaction mixture, and formulation into an efficacious therapeutic agent.

The term “optionally substituted” means optional substitution with the specified groups, radicals or moieties.

The term “purified”, “in purified form” or “in isolated and purified form” for a compound refers to the physical state of said compound after being isolated from a synthetic process (e.g., from a reaction mixture), or natural source or combination thereof. Thus, the term “purified”, “in purified form” or “in isolated and purified form” for a compound refers to the physical state of said compound after being obtained from a purification process or processes described herein or well known to the skilled artisan (e.g., chromatography, recrystallization and the like), in sufficient purity to be characterizable by standard analytical techniques described herein or well known to the skilled artisan.

It should also be noted that any carbon as well as heteroatom with unsatisfied valences in the text, schemes, examples and Tables herein is assumed to have the sufficient number of hydrogen atom(s) to satisfy the valences. And any one or more of these hydrogen atoms can be deuterium.

It should also be noted that in case of a discrepancy between the chemical name and structural formula for a specified compound, the description provided by the structural formula will be controlling.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

When any variable (e.g., aryl, heterocycle. R², etc.) occurs more than one time in any constituent or in Formula I, its definition on each occurrence is independent of its definition at every other occurrence.

As used herein, the term “composition” is intended to encompass a product comprising the specified ingredients in the specified amounts, as well as any product which results, directly or indirectly, from combination of the specified ingredients in the specified amounts.

Prodrugs and solvates of the compounds of the invention are also contemplated herein. A discussion of prodrugs is provided in T. Higuchi and V. Stella, Pro-drugs as Novel Delivery Systems (1987) 14 of the A.C.S. Symposium Series, and in Bioreversible Carriers in Drug Design, (1987) Edward B. Roche, ed., American Pharmaceutical Association and Pergamon Press. The term “prodrug” means a compound (e.g., a drug precursor) that is transformed in vivo to yield a compound of Formula (I) or a pharmaceutically acceptable salt, hydrate or solvate of the compound. The transformation may occur by various mechanisms (e.g., by metabolic or chemical processes), such as, for example, through hydrolysis in blood. A discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

For example, if a compound of Formula (I) or a pharmaceutically acceptable salt of the compound contains a carboxylic acid functional group, a prodrug can comprise an ester formed by the replacement of the hydrogen atom of the acid group with a group such as, for example, (C₁-C₈)alkyl, (C₂-C₁₂)alkanoyloxymethyl, 1-(alkanoyloxy)ethyl having from 4 to 9 carbon atoms, 1-methyl-1-(alkanoyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms. N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁-C₂)alkylamino(C₂-C₃)alkyl (such as β-dimethylaminoethyl), carbamoyl-(C₁-C₂)alkyl, N,N-di (C₁-C₂)alkylcarbamoyl-(C₁-C₂)alkyl and piperidino-, pyrrolidino- or morpholino(C₂-C₃)alkyl, and the like.

Similarly, if a compound of Formula (I) contains an alcohol functional group, a prodrug can be formed by the replacement of the hydrogen atom of the alcohol group with a group such as, for example, (C₁-C₆)alkanoyloxymethyl, 1-((C₁-C₆)alkanoyloxy)ethyl, 1-methyl-1-((C₁-C₆)alkanoyloxy)ethyl, (Cᵣ C₆)alkoxycarbonyloxymethyl, N—(C₁-C₆)alkoxycarbonylaminomethyl, succinoyl, (C₁-C₆)alkanoyl, α-amino(C₁-C₄)alkanyl, arylacyl and α-aminoacyl, or α-aminoacyl-α-aminoacyl, where each α-aminoacyl group is independently selected from the naturally occurring L-amino acids, P(O)(OH)₂, —P(O)(O(C₁-C₆)alkyl)₂ or glycosyl (the radical resulting from the removal of a hydroxyl group of the hemiacetal form of a carbohydrate), and the like.

If a compound of Formula (I) incorporates an amine functional group, a prodrug can be formed by the replacement of a hydrogen atom in the amine group with a group such as, for example, R-carbonyl, RO-carbonyl, NRR′-carbonyl where R and R′ are each independently (C₁-C₁₀)alkyl, (C₃-C₇) cycloalkyl, benzyl, or R-carbonyl is a natural α-aminoacyl or natural α-aminoacyl, C(OH)C(O)OY¹ wherein Y¹ is H, (C₁-C₆)alkyl or benzyl, —C(OY²)Y³ wherein Y² is (C₁-C₄) alkyl and Y³ is (C₁-C₆)alkyl, carboxy (C₁-C₆)alkyl, amino(C₁-C₄)alkyl or mono-N— or di-N,N—(C₁-C₆)alkylaminoalkyl, —C(Y⁴)Y⁵ wherein Y⁴ is H or methyl and Y⁵ is mono-N— or di-N,N—(C₁-C₆)alkylamino morpholino, piperidin-1-yl or pyrrolidin-1-yl, and the like.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may optionally be converted to a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS PharmSciTech., 5(1), article 12 (2004); and A. L. Bingham et al, Chem. Commun., 603-604 (2001). A typical, non-limiting, process involves dissolving the inventive compound in desired amounts of the desired solvent (organic or water or mixtures thereof) at a higher than ambient temperature, and cooling the solution at a rate sufficient to form crystals which are then isolated by standard methods. Analytical techniques such as, for example I. R. spectroscopy, show the presence of the solvent (or water) in the crystals as a solvate (or hydrate).

“Effective amount” or “therapeutically effective amount” is meant to describe an amount of compound or a composition of the present invention effective in inhibiting the above-noted diseases and thus producing the desired therapeutic, ameliorative, inhibitory or preventative effect.

The compounds of Formula I can form salts which are also within the scope of this invention. Reference to a compound of Formula I herein is understood to include reference to salts thereof, unless otherwise indicated. The term “salt(s)”, as employed herein, denotes acidic salts formed with inorganic and/or organic acids, as well as basic salts formed with inorganic and/or organic bases. In addition, when a compound of Formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the Formula I may be formed, for example, by reacting a compound of Formula I with an amount of acid or base, such as an equivalent amount, in a medium such as one in which the salt precipitates or in an aqueous medium followed by lyophilization.

Exemplary acid addition salts include acetates, ascorbates, benzoates, benzenesulfonates, bisulfates, borates, butyrates, citrates, camphorates, camphorsulfonates, fumarates, hydrochlorides, hydrobromides, hydroiodides, lactates, maleates, methanesulfonates, naphthalenesulfonates, nitrates, oxalates, phosphates, propionates, salicylates, succinates, sulfates, tartarates, thiocyanates, toluenesulfonates (also known as tosylates,) and the like. Additionally, acids which are generally considered suitable for the formation of pharmaceutically useful salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference thereto.

Exemplary basic salts include ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

All such acid salts and base salts are intended to be pharmaceutically acceptable salts within the scope of the invention and all acid and base salts are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, Orn-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁-C₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di (C₆₋₂₄)acyl glycerol.

Compounds of Formula I, and salts thereof, may exist in their tautomeric form (for example, as an amide or imino ether). All such tautomeric forms are contemplated herein as part of the present invention.

The compounds of Formula (I) may contain asymmetric or chiral centers, and, therefore, exist in different stereoisomeric forms. It is intended that all stereoisomeric forms of the compounds of Formula (I) as well as mixtures thereof, including racemic mixtures, form part of the present invention.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds of Formula (I) may be atropisomers (e.g., substituted biaryls) and are considered as part of this invention. Enantiomers can also be separated by use of chiral HPLC column.

It is also possible that the compounds of Formula (I) may exist in different tautomeric forms, and all such forms are embraced within the scope of the invention. Also, for example, all keto-enol and imine-enamine forms of the compounds are included in the invention.

All stereoisomers (for example, geometric isomers, optical isomers and the like) of the present compounds, such as those which may exist due to asymmetric carbons on various substituents, including enantiomeric forms (which may exist even in the absence of asymmetric carbons), rotameric forms, atropisomers, and diastereomeric forms, are contemplated within the scope of this invention. Also, for example, all keto-enol and imine-enamine forms of the compounds are included in the invention.) Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations. The use of the terms “salt”, “solvate”, “ester”, “prodrug” and the like, is intended to equally apply to the salt, solvate, ester and prodrug of enantiomers, stereoisomers, rotamers, tautomers, racemates or prodrugs of the inventive compounds.

The present invention also embraces isotopically-labelled compounds of the present invention which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, fluorine and chlorine and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl and ¹²³I, respectively.

Certain isotopically-labelled compounds of Formula (I) (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Certain isotopically-labelled compounds of Formula (I) can be useful for medical imaging purposes. E.g., those labeled with positron-emitting isotopes like ¹¹C or ¹⁸F can be useful for application in Positron Emission Tomography (PET) and those labeled with gamma ray emitting isotopes like ¹²³I can be useful for application in Single photon emission computed tomography (SPECT). Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Additionally, isotopic substitution at a site where epimerization occurs may slow or reduce the epimerization process and thereby retain the more active or efficacious form of the compound for a longer period of time. Isotopically labeled compounds of Formula (I), in particular those containing isotopes with longer half lives (T1/2>1 day), can generally be prepared by following procedures analogous to those disclosed in the Schemes and/or in the Examples herein below, by substituting an appropriate isotopically labeled reagent for a non-isotopically labeled reagent.

Polymorphic forms of the compounds of Formula I, and of the salts of the compounds of Formula I, are intended to be included in the present invention.

The present invention further includes the compounds of Formula (I) in all their isolated forms. For example, the above-identified compounds are intended to encompass all forms of the compounds such as, any solvates, hydrates, stereoisomers, and tautomers thereof.

The compounds according to the invention have pharmacological properties; in particular, the compounds of Formula I can be inhibitors of SCD1.

The following abbreviations are used below and have the following meanings: BINAP is racemic-2,2′-bis(diphenylphosphino)-1,1′-binaphthyl; BOC or Boc is tert-butyloxycarbonyl; CDI is carbonyl diimidazole; Ci/mmol is Curie/mmol; CSA is camphorsulfonic acid; DBPD is 2-(Di-t-butylphosphino)biphenyl, DBU is 1,8-diazabicyclo[5.4.0]undec-7-ene; DBN is 1,5-diazabicyclo[4.3.0]non-5-ene; DCC is dicyclohexylcarbodiimide; DCM is dichloromethane; Dibal-His diisobutylaluminum hydride; DIPEA is N,N-Diisopropylethylamine; DMAP is dimethylaminopyridine; DME is dimethoxyethane; DMF is dimethylformamide; dppf is diphenylphosphinoferrocene; EDCI is 1-(3-dimethylaminopropyl)-3-ethylcarbodiimide; EtOAc is ethyl acetate; FABMS is fast atom bombardment mass spectrometry; HATU is O-(7-azabenzotriazol-1-yl)-N,N,N′,N′-tetramethyluronium hexafluorophosphate; HOBT is 1-hydroxybenzotriazole; HOOBt is 3-hydroxy-1,2,3-benzotriazin-4(3H\-one; HPLC is high performance liquid chromatography; HRMS is high resolution mass spectrometry; Hunig's base is N,N-diisopropylethylamine; LAH is lithium aluminum hydride; LDA is lithium diisopropylamide; LRMS is low resolution mass spectrometry; m-CPBA is m-chloroperbenzoic acid; MeOH is methanol; NaBH(OAc)₃ is sodium triacetoxyborohydride; NaBH₄ is sodium borohydride; NaBH₃CN is sodium cyanoborohydride; NaHMDS is sodium hexamethyldisilazane; NH₄OAc is ammonium acetate; p-TsOH is p-toluenesulfonic acid; p-TsCl is p-toluenesulfonyl chloride; Pd(PPh₃)₄ is tetrakis(triphenylphosphine)palladium(0); PPTS is pyridinium p-toluenesulfonate; PYBROP is bromotripyrrolidinophosphoniunn hexafluorophosphate; SEM is β-(trimethylsilyl)ethoxy]methyl; SEMCI is β-(trimethylsilyl)ethoxymethyl chloride; TFA is trifluoroacetic acid; THF is tetrahydrofuran; TLC is thin-layer chromatography; TMAD is N,N,N′,N′-tetramethylazodicarboxamide; Tr is triphenylmethyl; and Tris is tris(hydroxymethyl)aminomethane.

The Compounds of Formula (I)

The present invention provides Compounds of Formula (I):

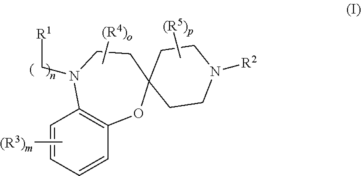

and pharmaceutically acceptable salts thereof, wherein R¹, R², R³, R⁴, R⁵, m, n, o, and p are defined above for the Compounds of Formula (I).

In one embodiment of the Compounds of Formula (I), R¹ is heteroaryl.

In another embodiment, R¹ is aryl.

In another embodiment, R¹ is phenyl.

In another embodiment, R¹ is phenyl substituted with one to four moieties, wherein said moieties are the same or different, and wherein said moieties are selected from the group consisting of alkyl, alkoxy, haloalkyl, halo, —CN, —C(O)—R⁶, —OCF₃, and R⁷. In particular instances, the moieties of said phenyl of R¹ are selected from the group consisting of alkyl, —CF₃, alkoxy, or —O-phenyl.

In yet another embodiment, R¹ is phenyl substituted with one moiety selected from the group consisting of alkyl, alkoxy, haloalkyl, halo, —CN, —C(O)—R⁶, —OCF₃, and R⁷. In particular instances, the moiety substituted on said phenyl of R¹ is alkyl, —CF₃, alkoxy, or —O-phenyl.

In one embodiment of the Compounds of Formula (I), R² is heteroaryl.

In another embodiment, said heteroaryl of R² comprises at least one nitrogen atom as a ring atom. For example, in some instances, said heteroaryl of R² is thiazole, pyridine, or pyridazine.

In yet another embodiment, said heteroaryl of R² is thiazole.

In another embodiment, R² is heteroaryl substituted with one to three moieties, which moieties are the same or different, and wherein said moieties are selected from the group consisting of alkyl, alkoxy, halo, haloalkyl, —CN, —C(O)—OH, —C(O)—O-alkyl, —C(O)-β-cycloalkyl, —C(O)—N(R¹²)₂, —OCF₃, aryl, heteroaryl, aryl substituted with alkyl, heteroaryl substituted by alkyl, and Z². In particular instances, said heteroaryl of R² is substituted with one —CN, —C(O)—N(R¹²)₂, —C(O)—OH, heteroaryl substituted with alkyl, or Z². For example in some instances, said heteroaryl of R² is substituted with one —CN. In other instances, said heteroaryl of R² is substituted with one —C(O)—N(R¹²)₂. In other instances, said heteroaryl of R² is substituted with one —C(O)—N(R¹²)₂, wherein at least one R¹² is H.

In another embodiment, R² is heteroaryl substituted with one Z², wherein Z² is

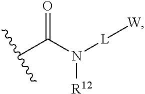

wherein L is a direct bond such that W is bonded directly to the illustrated N atom of —N(R¹²)—, or L is —(CH₂)ₓ—, —CH₂—C(H)(OH)—, or —CH₂—C(H)(OH)—CH₂—;

W is —C(O)OR¹³, —C(O)NR¹², —S(O)alkyl, —S(O)₂alkyl, —CF₃, —C(H)(OH)—CH₂OH, —CH₂OH, —C(H)(CH₃)OH, cycloalkyl, aryl, heteroaryl, heterocyclyl, wherein said cycloalkyl, aryl, heteroaryl, or heterocyclyl of W is unsubstituted or substituted with one to three moieties, wherein said moieties are selected from the group consisting of alkyl, hydroxyl, alkoxy, halo, —CF₃, —OCF₃, or —CN; and

with the proviso that when W is —C(O)OR¹³, —C(O)NR¹², —S(O)alkyl, —S(O)₂alkyl, —CF₃, —C(H)(OH)—CH₂OH, or —CH₂OH or, then L must be —(CH₂)ₓ—, —CH₂—C(H)(OH)—, or —CH₂—C(H)(OH)—CH₂—.

In some instances wherein R² is heteroaryl substituted with one Z², R¹² of Z² is H.

In other instances, R¹² of Z² is H;

L is —(CH)ₓ—; and

W is —C(O)OR¹³, —C(O)N(R¹²)₂, —C(H)(OH)—CH₂OH, —CH₂OH, —C(H)(CH₃)OH, cycloalkyl, or heterocyclyl.

In one embodiment of the Compounds of Formula (I), n is 1.

In another embodiment of the Compounds of Formula (I), R³ is halo and m is 1. In some instances, for example, R³ is fluoro and m is 1.

In another embodiment, m is 0.

In another embodiment of the Compounds of Formula (I), o is 0.

In another embodiment of the Compounds of Formula (I), p is 0.

In yet another embodiment of the Compounds of Formula (I), both o and p are 0.

In another aspect of the Compounds of Formula (I), R¹ is phenyl substituted with one alkyl, —CF₃, alkoxy, or —O-phenyl;

R² is heteroaryl substituted with one to three moieties, which moieties are the same or different, and wherein said moieties are selected from the group consisting of alkyl, alkoxy, halo, haloalkyl, —CN, —C(O)—OH, —C(O)—O-alkyl, —C(O)-β-cycloalkyl, —C(O)—N(R¹²)₂, —OCF₃, aryl, heteroaryl, aryl substituted with alkyl, heteroaryl substituted by alkyl, and Z²;

R³ is halo;

m is 0 or 1;

n is 1;

o is 0; and

p is 0.

In certain embodiments of this aspect, said heteroaryl of R² is substituted with one —CN, —C(O)—N(R¹²)₂, —C(O)—OH, heteroaryl substituted with alkyl, or Z². For example, in certain instances, the heteroaryl of R² is substituted with one —CN. In other instances, the heteroaryl of R² is substituted with one —C(O)—N(R¹²)₂ and at least one R¹² is H. In other embodiments of this aspect, said heteroaryl of R² is substituted with one Z², wherein R¹² of said Z² is H, L is —(CH)ₓ— and W is —C(O)OR¹³, —C(O)N(R¹²)₂, —C(H)(OH)—CH₂OH, —CH₂OH, —C(H)(CH₃)OH, cycloalkyl, or heterocyclyl.

In other embodiments of this aspect, said heteroaryl of R² comprises at least one nitrogen atom as a ring member. For example, said heteroaryl can be thiazole, pyridine, or pyridazine.

In one embodiment of the Compounds of Formula (I), R¹, R², R³, R⁴, R⁵, R⁶, R⁸, R⁹, R¹⁰, R¹¹, R¹², R¹³, W, Y, Z¹, Z² n, o, and p are selected independently from each other.

In another embodiment, a Compound of Formula (I) is in purified form.

Non-limiting examples of the Compounds of Formula (I) include compounds I-184 and pharmaceutically acceptable salts of such compounds as set forth below in Tables 1, 2, and 3 in the Examples section.

Compounds B1-B45, whose structural formulas and chemical names are listed below are not part of the present invention.

| | | |
|---|---|---|
| 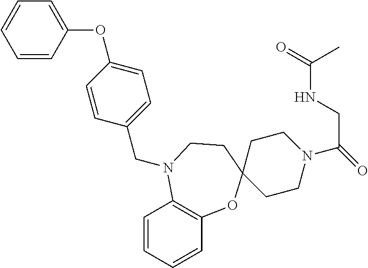 | B1 | N-(2-oxo-2-(5-(4-phenoxybenzyl)-4,5- dihydro-3H-spiro[benzo[b][1,4]oxazepine- 2,4′-piperidine]-1′-yl)ethyl)acetamide |
| 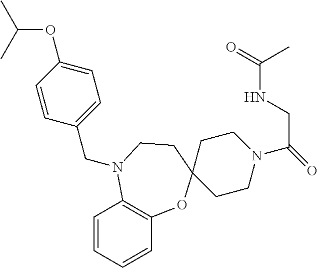 | B2 | N-(2-(5-(4-isopropoxybenzyl)-4,5-dihydro- 3H-spiro[benzo[b][1,4]oxazepine-2,4′- piperidine]-1′-yl)-2-oxoethyl)acetamide |
| 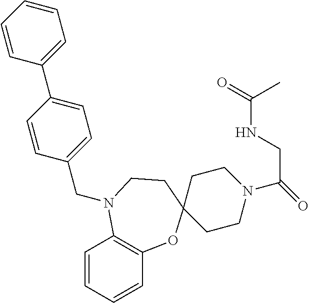 | B3 | N-(2-(5-(biphenyl-4-ylmethyl)-4,5-dihydro- 3H-spiro[benzo[b][1,4]oxazepine-2,4′- piperidine]-1′-yl)-2-oxoethyl)acetamide |
| 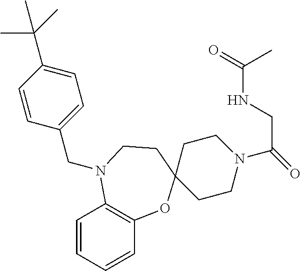 | B4 | N-(2-(5-(4-tert-butylbenzyl)-4,5-dihydro-3H- spiro[benzo[b][1,4]oxazepine-2,4′-piperidine]- 1′-yl)-2-oxoethyl)acetamide |
| 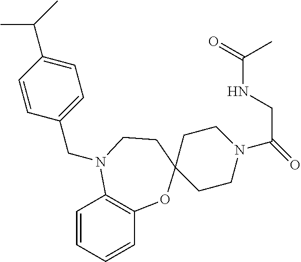 | B5 | N-(2-(5-(4-isopropylbenzyl)-4,5-dihydro-3H- spiro[benzo[b][1,4]oxazepine-2,4′-piperidine]- 1′-yl)-2-oxoethyl)acetamide |
| 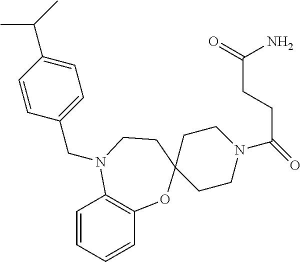 | B6 | 4-(5-(4-isopropylbenzyl)-4,5-dihydro-3H- spiro[benzo[b][1,4]oxazepine-2,4′-piperidine]- 1′-yl)-4-oxobutanamide |
| 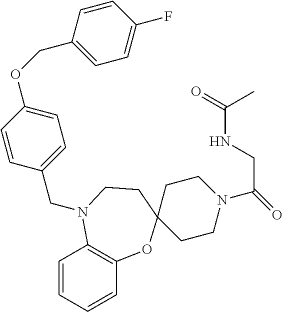 | B7 | N-(2-(5-(4-(4-fluorobenzyloxy)benzyl)-4,5- dihydro-3H-spiro[benzo[b][1,4]oxazepine- 2,4′-piperidine]-1′-yl)-2-oxoethyl)acetamide |
| 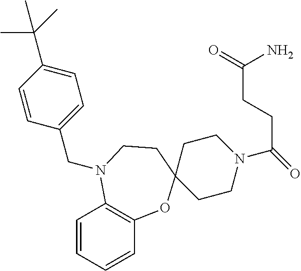 | B8 | 4-(5-(4-tert-butylbenzyl)-4,5-dihydro-3H- spiro[benzo[b][1,4]oxazepine-2,4′-piperidine]- 1′-yl)-4-oxobutanamide |
| 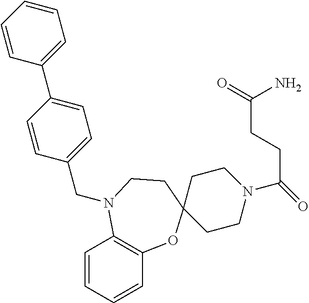 | B9 | 4-(5-(biphenyl-4-ylmethyl)-4,5-dihydro-3H- spiro[benzo[b][1,4]oxazepine-2,4′-piperidine]- 1′-yl)-4-oxobutanamide |
| 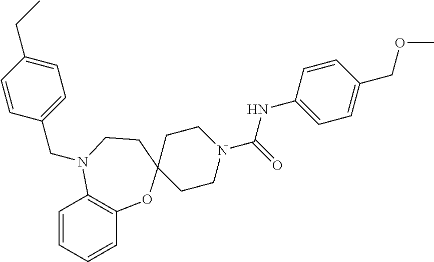 | B10 | 5-(4-ethylbenzyl)-N-(4-(methoxymethyl) phenyl)-4,5-dihydro-3H-spiro[benzo[b][1,4] oxazepine-2,4′-piperidine]-1′-carboxamide |
| 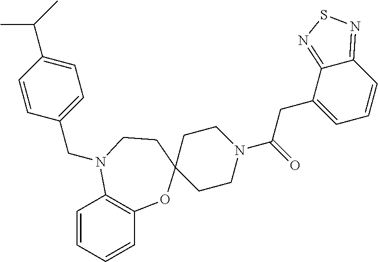 | B11 | 2-(benzo[c][1,2,5]thiadiazol-4-yl)-1-(5-(4- isopropylbenzyl)-4,5-dihydro-3H- spiro[benzo[b][1,4]oxazepine-2,4′- piperidine]-1′-yl)ethanone |
| 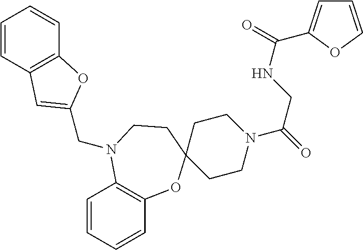 | B12 | N-(2-(5-(benzofuran-2-ylmethyl)-4,5-dihydro- 3H-spiro[benzo[b][1,4]oxazepine-2,4′- piperidine]-1′-yl)-2-oxoethyl)furan-2- carboxamide |
| 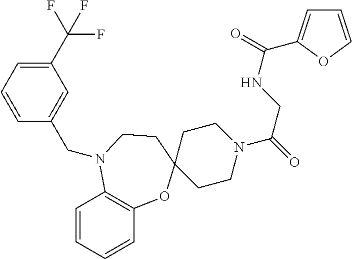 | B13 | N-(2-oxo-2-(5-(3-(trifluoromethyl)benzyl)-4,5- dihydro-3H-spiro[benzo[b][1,4]oxazepine- 2,4′-piperidine]-1′-yl)ethyl)furan-2- carboxamide |
| 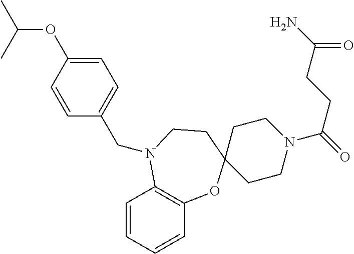 | B14 | 4-(5-(4-isopropoxybenzyl)-4,5-dihydro-3H- spiro[benzo[b][1,4]oxazepine-2,4′-piperidine]- 1′-yl)-4-oxobutanamide |
| 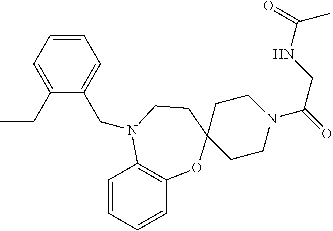 | B15 | N-(2-(5-(2-ethylbenzyl)-4,5-dihydro-3H- spiro[benzo[b][1,4]oxazepine-2,4′-piperidine]- 1′-yl)-2-oxoethyl)acetamide |
| 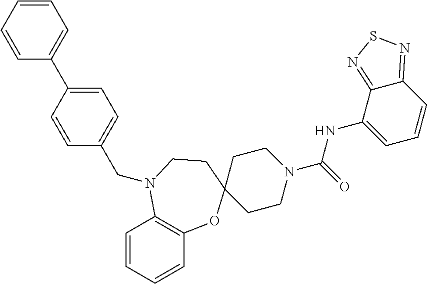 | B16 | N-(benzo[c][1,2,5]thiadiazol-4-yl)-5- (biphenyl-4-ylmethyl)-4,5-dihydro-3H- spiro[benzo[b][1,4]oxazepine-2,4′- piperidine]-1′-carboxamide |
| 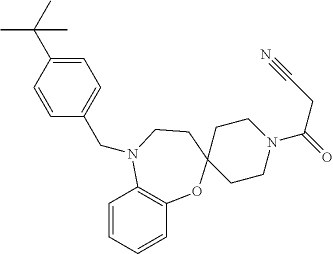 | B17 | 3-(5-(4-tert-butylbenzyl)-4,5-dihydro-3H- spiro[benzo[b][1,4]oxazepine-2,4′-piperidine]- 1′-yl)-3-oxopropanenitrile |
| 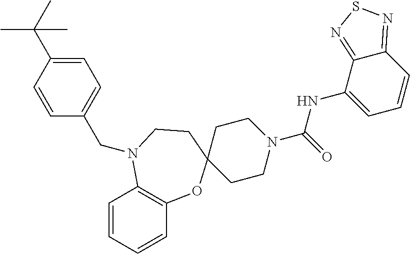 | B18 | N-(benzo[c][1,2,5]thiadiazol-4-yl)-5-(4-tert- butylbenzyl)-4,5-dihydro-3H- spiro[benzo[b][1,4]oxazepine-2,4′- piperidine]-1′-carboxamide |
| 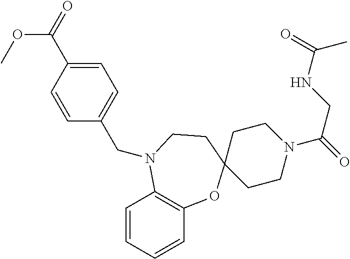 | B19 | methyl 4-((1′-(2-acetamidoacetyl)-3H- spiro[benzo[b][1,4]oxazepine-2,4′-piperidine]- 5(4H)-yl)methyl)benzoate |
| 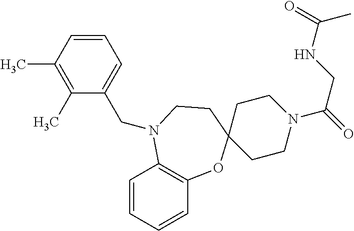 | B20 | N-(2-(5-(2,3-dimethylbenzyl)-4,5-dihydro-3H- spiro[benzo[b][1,4]oxazepine-2,4′-piperidine]- 1′-yl)-2-oxoethyl)acetamide |
| 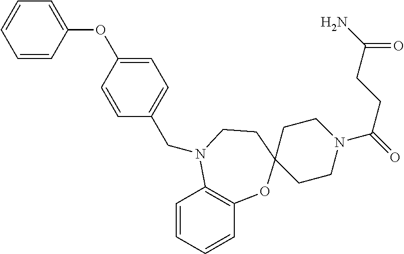 | B21 | 4-oxo-4-(5-(4-phenoxybenzyl)-4,5-dihydro- 3H-spiro[benzo[b][1,4]oxazepine-2,4′- piperidine]-1′-yl)butanamide |
| 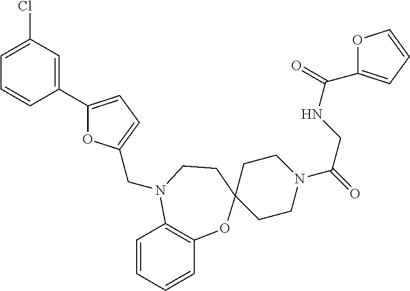 | B22 | N-(2-(5-((5-(3-chlorophenyl)furan-2-yl)methyl)- 4,5-dihydro-3H-spiro[benzo[b][1,4]oxazepine- 2,4′-piperidine]-1′-yl)-2-oxoethyl)furan-2- carboxamide |
| 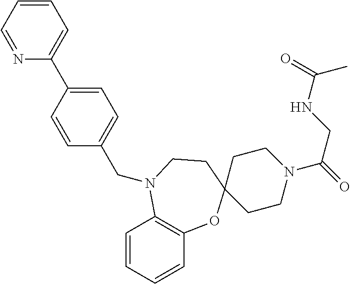 | B23 | N-(2-oxo-2-(5-(4-(pyridin-2-yl)benzyl)-4,5- dihydro-3H-spiro[benzo[b][1,4]oxazepine- 2,4′-piperidine]-1′-yl)ethyl)acetamide |
| 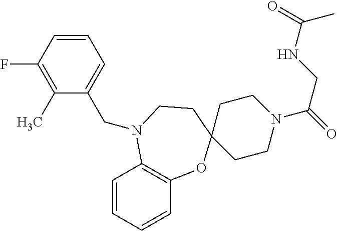 | B24 | N-(2-(5-(3-fluoro-2-methylbenzyl)-4,5- dihydro-3H-spiro[benzo[b][1,4]oxazepine- 2,4′-piperidine]-1′-yl)-2-oxoethyl)acetamide |
| 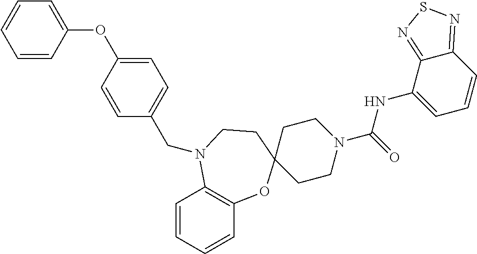 | B25 | N-(benzo[c][1,2,5]thiadiazol-4-yl)-5-(4- phenoxybenzyl)-4,5-dihydro-3H- spiro[benzo[b][1,4]oxazepine-2,4′- piperidine]-l′-carboxamide |
| 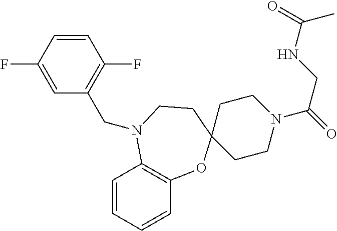 | B26 | N-(2-(5-(2,5-difluorobenzyl)-4,5-dihydro-3H- spiro[benzo[b][1,4]oxazepine-2,4′-piperidine]- 1′-yl)-2-oxoethyl)acetamide |
| 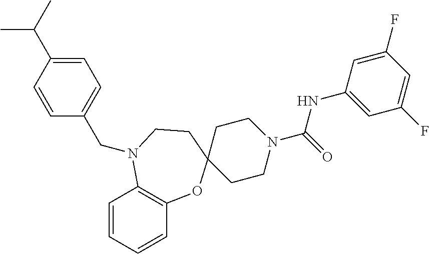 | B27 | N-(3,5-difluorophenyl)-5-(4-isopropylbenzyl)- 4,5-dihydro-3H-spiro[benzo[b][1,4]oxazepine- 2,4′-piperidine]-1′-carboxamide |
| 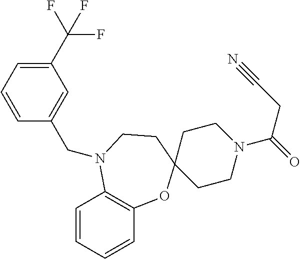 | B28 | 3-oxo-3-(5-(3-(trifluoromethyl)benzyl)-4,5- dihydro-3H-spiro[benzo[b][1,4]oxazepine- 2,4′-piperidine]-1′-yl)propanenitrile |
| 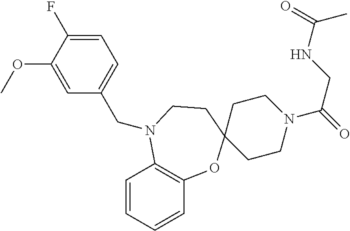 | B29 | N-(2-(5-(4-fluoro-3-methoxybenzyl)-4,5- dihydro-3H-spiro[benzo[b][1,4]oxazepine- 2,4′-piperidine]-1′-yl)-2-oxoethyl)acetamide |
| 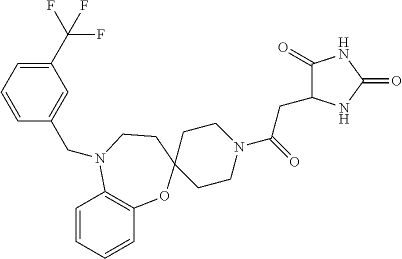 | B30 | 5-(2-oxo-2-(5-(3-(trifluoromethyl)benzyl)-4,5- dihydro-3H-spiro[benzo[b][1,4]oxazepine- 2,4′-piperidine]-1′-yl)ethyl)imidazolidine-2,4- dione |
| 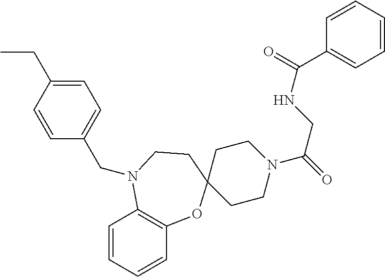 | B31 | N-(2-(5-(4-ethylbenzyl)-4,5-dihydro-3H- spiro[benzo[b][1,4]oxazepine-2,4′-piperidine]- 1′-yl)-2-oxoethyl)benzamide |
| 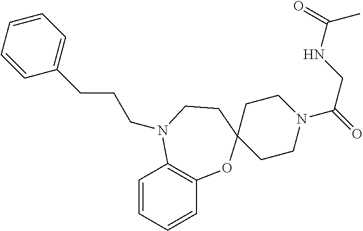 | B32 | N-(2-oxo-2-(5-(3-phenylpropyl)-4,5-dihydro- 3H-spiro[benzo[b][1,4]oxazepine-2,4′- piperidine]-1′-yl)ethyl)acetamide |
| 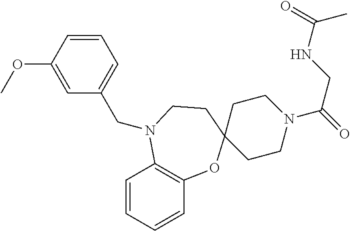 | B33 | N-(2-(5-(3-methoxybenzyl)-4,5-dihydro-3H- spiro[benzo[b][1,4]oxazepine-2,4′-piperidine]- 1′-yl)-2-oxoethyl)acetamide |
| 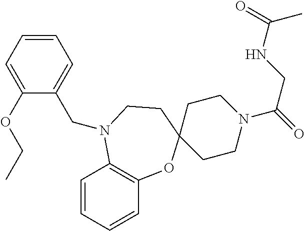 | B34 | N-(2-(5-(2-ethoxybenzyl)-4,5-dihydro-3H- spiro[benzo[b][1,4]oxazepine-2,4′-piperidine]- 1′-yl)-2-oxoethyl)acetamide |
| 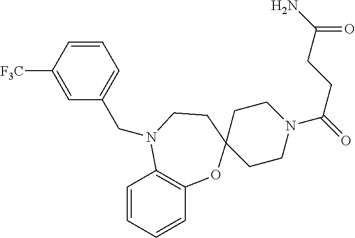 | B35 | 4-oxo-4-(5-(3-(trifluoromethyl)benzyl)-4,5- dihydro-3H-spiro[benzo[b][1,4]oxazepine- 2,4′-piperidine]-1′-yl)butanamide |
| 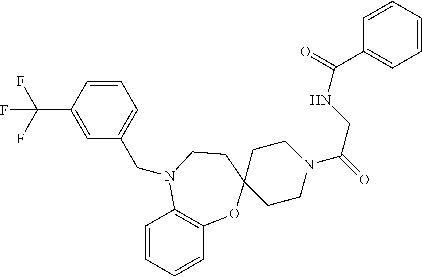 | B36 | N-(2-oxo-2-(5-(3-(trifluoromethyl)benzyl)-4,5- dihydro-3H-spiro[benzo[b][1,4]oxazepine- 2,4′-piperidine]-1′-yl)ethyl)benzamide |
| 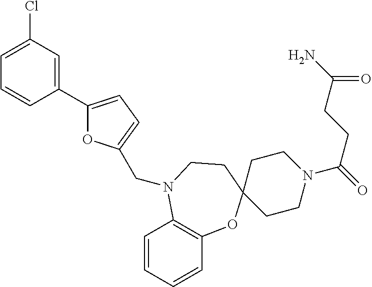 | B37 | 4-(5-((5-(3-chlorophenyl)furan-2-yl)methyl)- 4,5-dihydro-3H-spiro[benzo[b][1,4]oxazepine- 2,4′-piperidine]-1′-yl)-4-oxobutanamide |
| 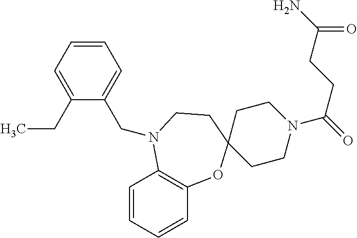 | B38 | 4-(5-(2-ethylbenzyl)-4,5-dihydro-3H- spiro[benzo[b][1,4]oxazepine-2,4′-piperidine]- 1′-yl)-4-oxobutanamide |
| 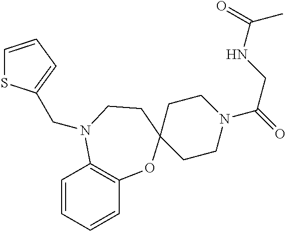 | B39 | N-(2-oxo-2-(5-(thiophen-2-ylmethyl)-4,5- dihydro-3H-spiro[benzo[b][1,4]oxazepine- 2,4′-piperidine]-1′-yl)ethyl)acetamide |
| 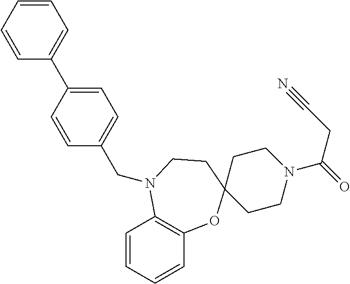 | B40 | 3-(5-(biphenyl-4-ylmethyl)-4,5-dihydro-3H- spiro[benzo[b][1,4]oxazepine-2,4′-piperidine]- 1′-yl)-3-oxopropanenitrile |
| 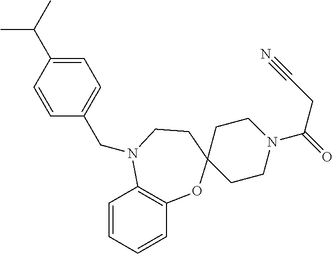 | B41 | 3-(5-(4-isopropylbenzyl)-4,5-dihydro-3H- spiro[benzo[b][1,4]oxazepine-2,4′-piperidine]- 1′-yl)-3-oxopropanenitrile |
| 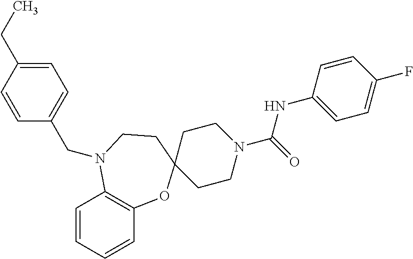 | B42 | 5-(4-ethylbenzyl)-N-(4-fluorophenyl)-4,5- dihydro-3H-spiro[benzo[b][1,4]oxazepine- 2,4′-piperidine]-1′-carboxamide |
| 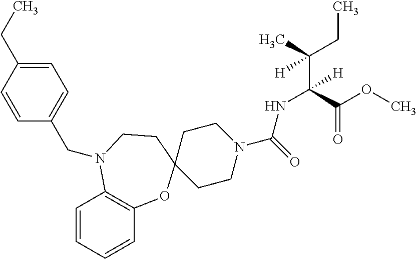 | B43 | (2S,3S)-methyl 2-(5-(4-ethylbenzyl)-4,5- dihydro-3H-spiro[benzo[b][1,4]oxazepine- 2,4′-piperidine]-1′-ylcarboxamido)-3- methylpentanoate |
| 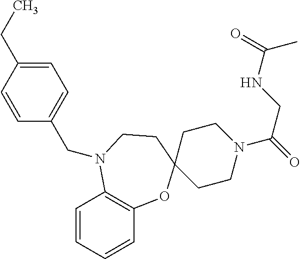 | B44 | N-(2-(5-(4-ethylbenzyl)-4,5-dihydro-3H- spiro[benzo[b][1,4]oxazepine-2,4′-piperidine]- 1′-yl)-2-oxoethyl)acetamide |
| 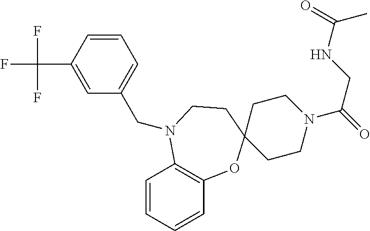 | B45 | N-(2-oxo-2-(5-(3-(trifluoromethyl)benzyl)-4,5- dihydro-3H-spiro[benzo[b][1,4]oxazepine- 2,4′-piperidine]-1′-yl)ethyl)acetamide |

### Methods For Making the Compounds of Formula (I)

The Compounds of Formula (I) may be prepared from known or readily prepared starting materials, following methods known to one skilled in the art of organic synthesis. Methods useful for making the Compounds of Formula (I) are set forth in the Examples below and are generalized in Schemes 1-3. Alternative synthetic pathways and analogous structures will be apparent to those skilled in the art of organic synthesis. All stereoisomers and tautomeric forms of the compounds are contemplated.

The starting materials and reagents described in the Examples and in Schemes 1-3 below are either available from commercial suppliers such as Sigma-Aldrich (St. Louis, Mo.) and Acros Organics Co. (Fair Lawn, N.J.), or can be prepared using methods well-known to one skilled in the art of organic synthesis.

One skilled in the art of organic synthesis will also recognize that the synthesis of the Compounds of Formula (I) may require protection of certain functional groups (i.e., derivatization for the purpose of chemical compatibility with a particular reaction condition). Suitable protecting groups for the various functional groups of these compounds and methods for their installation and removal can be found in Greene et al., Protective Groups in Organic Synthesis, Wiley-Interscience, New York, (1999).

The starting materials used and the intermediates prepared using the methods set forth in the schemes above may be isolated and purified if desired using conventional techniques, including but not limited to filtration, distillation, crystallization, chromatography and alike. Such materials can be characterized using conventional means, including physical constants and spectral data.

Scheme 1 illustrates the preparation of benzo-fused oxazepine starting materials A-4 from hydroxyphenylethanones.

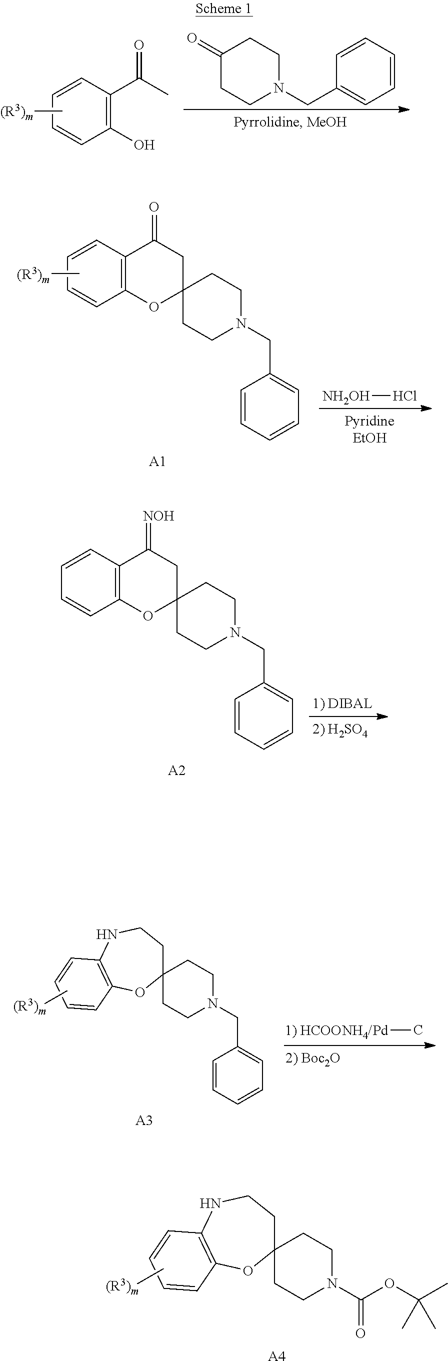

The preparation of Intermediate A4 is modified from the procedure described in Willand et al., Synthesis and Structural Studies of a Novel Scaffold for Drug Discovery: A 4,5-dihydro-3H-spiro[1,5-benzoxazepine-2,4′-piperidine, 45 Tetrahedron Lett 1051-1054 (2004), hereinafter “Willand et al.” Intermediate A1 is synthesized using substituted hydroxyphenylethanones as starting materials. The crude product A1 is converted into the corresponding oxime A2 using hydroxylamine hydrochloride. Reductive rearrangement of oxime A2 by DIBAL gives intermediate A3. After removing the benzyl protecting group of intermediate using palladium-catalyzed reduction, the crude, de-protected product is reacted with Boc₂O without purification to give intermediate A4.

In some embodiments in the synthesis of intermediate A3, a side product, intermediate A3′

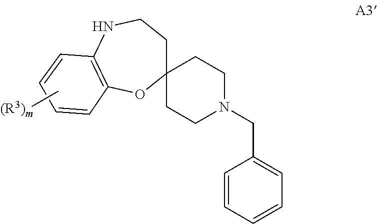

is recovered in addition to the fully reduced intermediate, A3. Treating the mixture of A3′ and A3 with lithium aluminum hydride in a suitable solvent, e.g., tetrahydrofuran, converts the mixture to the fully reduced intermediate A3. Scheme 2 illustrates the alkylation of N-1 of the benzo-fused oxazepine intermediate A4 to give the intermediate A5, and the acylation of N-6 of the benzo-fused oxazepine core. The acylation reaction can be used in the preparation of Compounds of the Formula (I), wherein R² is C(O)Y, wherein Y is alkyl or cycloalkyl, or wherein R² is

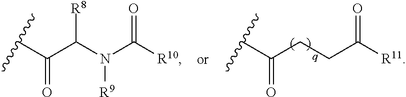

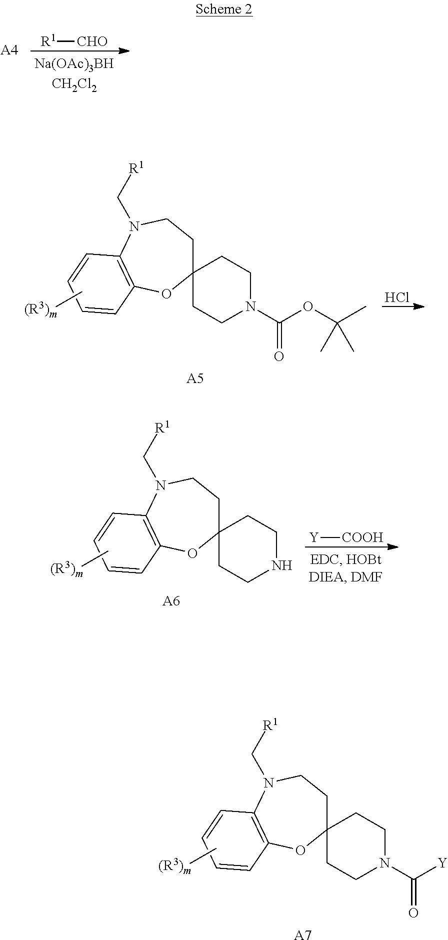

The N-1 of the benzo-fused oxazepine core of intermediate A4 can be alkylated with an R¹ alkylene group, e.g., R¹ methylene, by reductive amination to afford the BOC-protected intermediate A5. Intermediate A5 is de-protected under acidic conditions, and the free amine can be coupled with carboxylic acids give compounds A7.

Scheme 3 illustrates a method for preparing a compound of the Formula (I) wherein R² is substituted or unsubstituted aryl or heteroaryl. The displacement reaction of A6 to A8 can be performed under microwave conditions, or under Buchwald coupling conditions such as those described in Surry et al., Biaryl Phosphane Ligands in Palladium-Catalyzed Amination, 47 Angew. Chem. Int. Ed. 6338-6361 (2008).

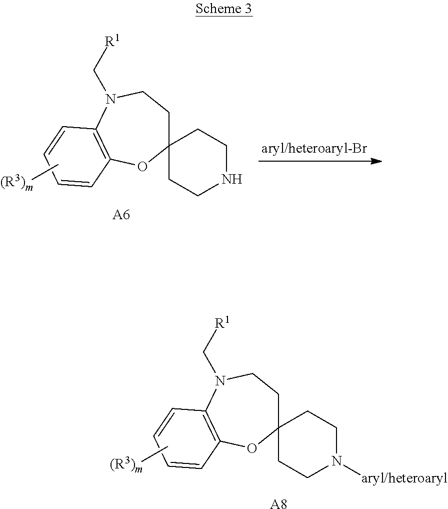

### EXAMPLES

### General Methods

Solvents, reagents, and intermediates that are commercially available were used as received. Reagents and intermediates that are not commercially available were prepared in the manner as described below. ¹H NMR spectra were obtained on a Varian AS-400 (400 MHz) or Varian 500 MHz NMR and are reported as ppm down field from Me₄Si with number of protons, multiplicities, and coupling constants in Hz indicated parenthetically.

Where LC/MS data are presented, analyses was performed using an Applied Biosystems API-100 mass spectrometer and Shimadzu SCL-10A LC column: Altech platinum C18, 3 micron, 33 mm×7 mm ID; 1 ml/min; gradient flow: 0 min-10% CH₃CN, 5 min-95% CH₃CN, 7 min-95% CH₃CN, 7.5 min-10% CH₃CN, 9 min-stop. The retention time and observed parent ion are given. MS data were obtained using Agilent Technologies LC/MSD SL or 1100 series LC/MSD mass spectrometer. The molecular ion peaks and retention time data for compounds 1-184 are provided in Tables 1-3 below.

Final compounds were purified by PrepLC using the column of Varian Pursuit XRs C18 10 mm 250×21.2 mm and an eluent mixture of mobile phase A and B. The mobile phase A is composed of 0.1% TFA in H₂O and the mobile phase B is composed of CH₃CN (95%)/H₂O (5%)/TFA (0.1%). The mixture of mobile phase A and B was eluted through the column at a flow rate of 20 mL/min at room temperature. The purity of all the final discrete compounds was checked by LCMS using a Higgins Haisil HL C18 5 mm 150×4.6 mm column and an eluent mixture of mobile phase A and B, wherein mobile phase A is composed of 0.1% TFA in H₂O and the mobile phase B is composed of CH₃CN (95%)/H₂O (5%)/TFA (0.1%). The column was eluted at a flow rate of 3 mL/min at a temperature of 60 C. Intermediate compounds were characterized by LCMS using a Higgins Haisil HL C18 5 mm 50×4.6 mm column and an eluent mixture of mobile phase A and B, wherein mobile phase A is composed of 0.1% TFA in H₂O and the mobile phase B is composed of CH3CN (95%)/H2O (5%)/TFA (0.1%). The column was eluted at a flow rate of 3 mL/min at a column temperature of 60 C.

### Example 1

### Preparation of Intermediates of Type A1

### Synthesis of 1′-benzyl-5-fluorospiro[chroman-2,4′-piperidin]-4-one (Int-1a)

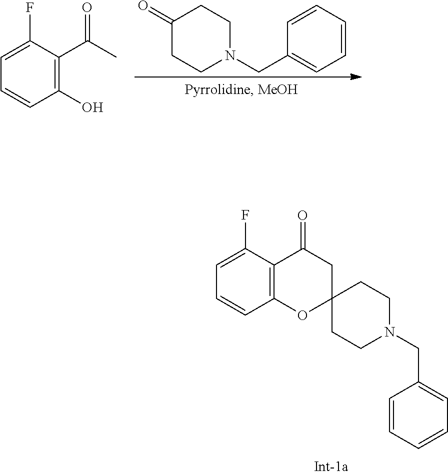

1-(2-fluoro-6-hydroxyphenyl)ethanone (0.46 gm, 3.0 mmole) and 1-benzylpiperidin-4-one (0.68 gm, 3.6 mmole, 1.2 eq) were dissolved in methanol (3.0 mL). Pyrrolidine (125 μL, 0.50 eq) was added and the mixture was stirred at 60° C. overnight. The solvent was evaporated under vacuum. The residue was dissolved in ethyl acetate and washed with saturated 1N Na₂CO₃ and water, dried with MgSO₄, and concentrated to afford Int-1a as a sticky, yellowish solid. LCMS: 326.2 (M+1).

### Synthesis of 1′-benzyl-4-fluorospiro[chroman-2,4′-piperidin]-4-one (Int-1b)

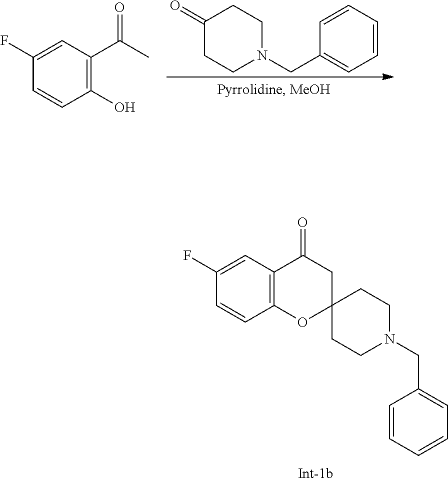

Int-1 b was synthesized in a similar fashion as Int-1a using 1-(3-fluoro-6-hydroxyphenyl)ethanone as starting material. LCMS: 326.2 (M+1).

### Synthesis of 1′-benzyl-4-fluorospiro[chroman-2,4′-piperidin]-4-one (Int-1c)

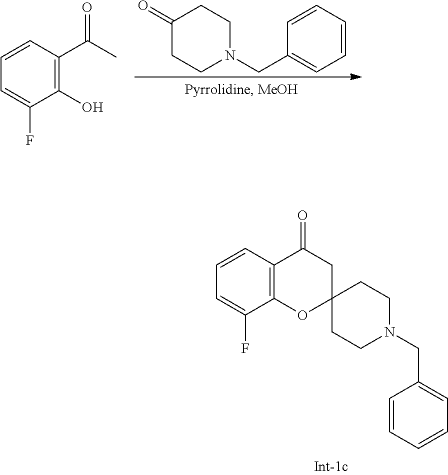

Int-1c was synthesized in a similar fashion as Int-1a using 1-(3-fluoro-2-hydroxyphenyl)ethanone as starting material. LCMS: 326.2 (M+1).

### Synthesis of 1′-benzyl-4-chlorospiro[chroman-2,4′-piperidin]-4-one (Int-1d)

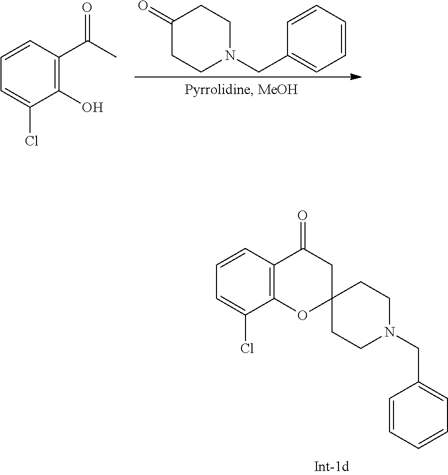

It-1d was synthesized in a similar fashion as It-1a using 1-(3-chloro-2-hydroxyphenyl)ethanone as starting material. LCMS: 342.1 (M+1). Proton NMR (400 MHz in DMSO-d₆): δ7.

### Example 2

### Preparation of Intermediates of Type A2

### Synthesis of 1′-benzyl-5-fluorospiro[chroman-2,4′-piperidin]-4-one oxime (Int-2a)

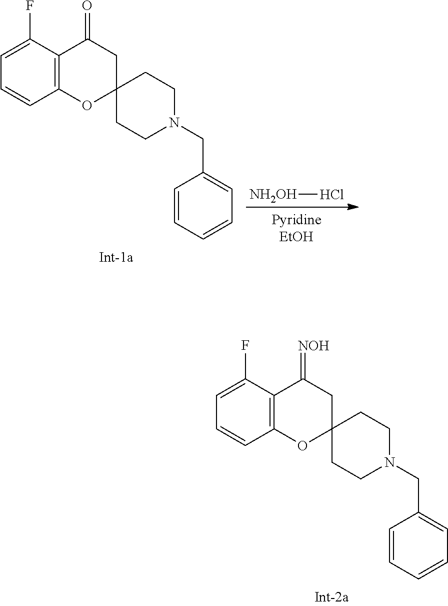

Int-1a (1.5 gm, 4.62 mmole) and hydroxylamine hydrochloride (0.70 gm, 10.0 mmole, 2.2 eq) were dissolved in a mixture of ethanol (10.0 mL) and pyridine (1.0 mL). The reaction was refluxed overnight. After cooling to room temperature, water (100 mL) was mixed with the crude reaction mixture. The mixture was settled for 30 minutes at room temperature. The precipitate was filtered out and washed with cold water. After drying under high vacuum overnight. Int-2a was obtained as a slightly off-white powder.

### Synthesis of 1′-benzyl-4-fluorospiro[chroman-2,4′-piperidin]-4-one oxime (Int-2b)

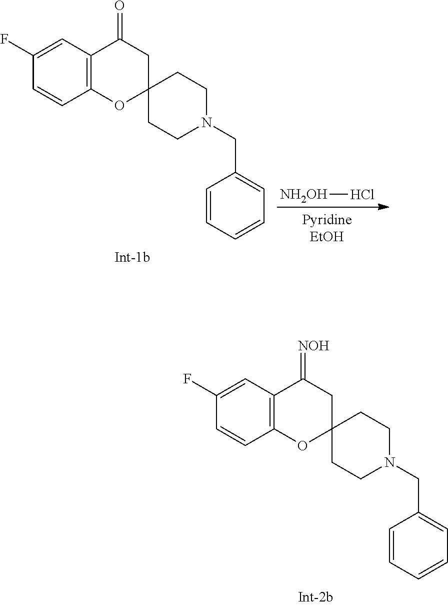

Int-2b was synthesized in a similar fashion as Int-2a using Int-1b as starting material. LCMS: 341.2 (M+1).

### Synthesis of 1′-benzyl-8-fIuorospiro[chroman-2,4′-piperidin]-4-one oxime (Int-2c)

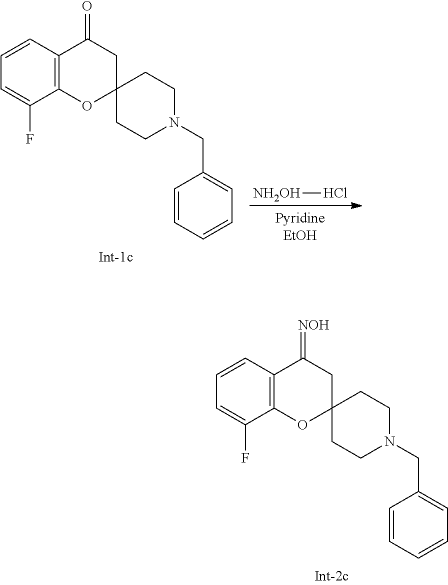

Int-2c was synthesized in a similar fashion as Int-2a using Int-1c as starting material. LCMS: 341.2 (M+1).

### Synthesis of 1′-benzyl-8-chlorospiro[chroman-2,4′-piperidin]-4-one oxime (Int-2d)

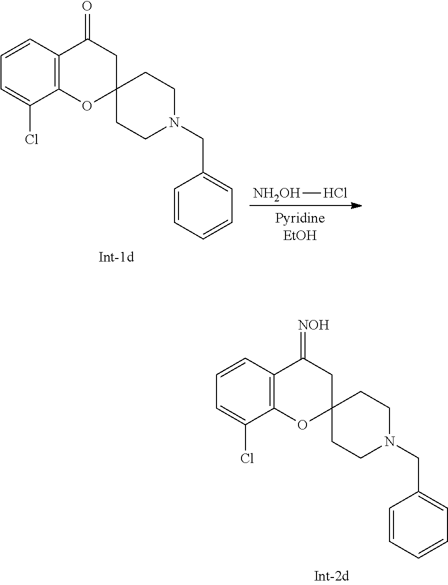

Int-2d was synthesized in a similar fashion as Int-2d using Int-1d as starting material. LCMS: 366.1 (M+1).

### Example 3

### Preparation of Intermediates of Type A3

### Synthesis of 1′-benzyl-6-fluoro-4,5-dihydro-3H-spiro[benzo[b]-[1,4]oxazepine-2,4′-piperidine] (Int-3a)

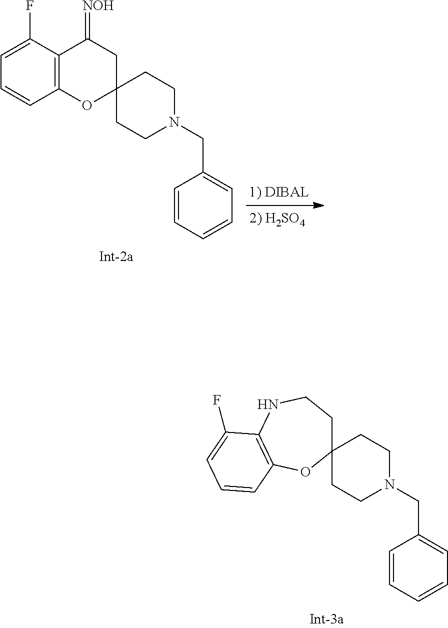

In an oven-dried flask, Int-2a (0.36 gm, 1.1 mmole) was dissolved in dry dichloromethane (2.28 mL) and flushed with argon. The solution was stirred at 0° C. for 20 minutes followed by slow addition of diisobutylaluminum hydride solution (1.0 N in dichloromethane, 6.4 mL, 5.8 eq). The mixture was stirred at 0° C. for 4 hours. The reaction was quenched by sequential addition of methanol (1.0 mL), water (1.0 mL), and 20% sulfuric acid (5.8 mL). The mixture was stirred at room temperature for 20 minutes. The crude mixture was adjusted to pH 9 by KOH (5N). The precipitate was filtered off, and the filtrate was extracted with ethyl acetate. The organic phase was collected, dried with MgSO₄, and evaporated to afford the crude product as yellowish oil. This crude product was used in the following steps without further purification. LCMS: 327.2 (M+1).

### Synthesis of 1′-benzyl-7-fluoro-4,5-dihydro-3H-spiro[benzo[b]-[1,4]oxazepine-2,4′-piperidine] (Int-3b)

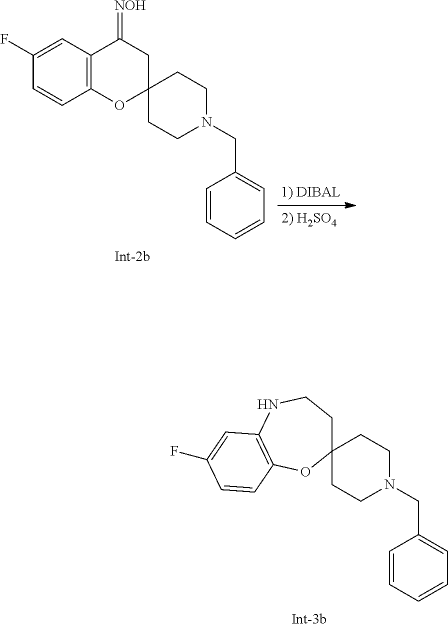

Int-3b was synthesized in a similar fashion as Int-3a using Int-2b as starting material. LCMS: 327.2 (M+1).

### Synthesis of 1′-benzyl-9-fluoro-4,5-dihydro-3H-spiro[benzo[b]1,4]oxazepine-2,4′-piperidine] (Int-3c)

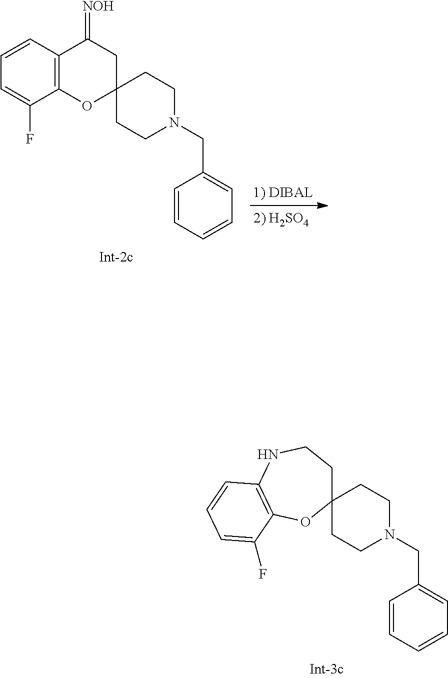

Int-3c was synthesized in a similar fashion as Int-3a using Int-2c as starting material. LCMS: 327.2 (M+1).

### Synthesis of 1′-benzyl-9-chloro-4,5-dihydro-3H-spiro[benzo[b][1,4]oxazepine-2,4′-piperidine] (Int-3d)

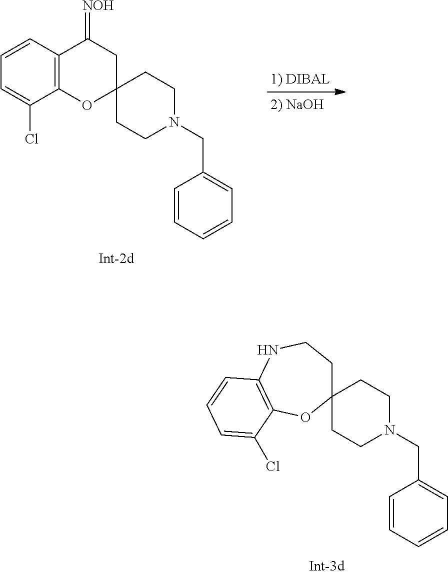

Int-3d was synthesized in a similar fashion as Int-3a using Int-2d as starting material. The product was worked up under basic conditions. LCMS: 327.2 (M+1).

### Synthesis of 1′-benzyl-4,5-dihydro-3H-spiro[benzo[b][1,4]oxazepine-2,4′-piperidine] (Int-3e)

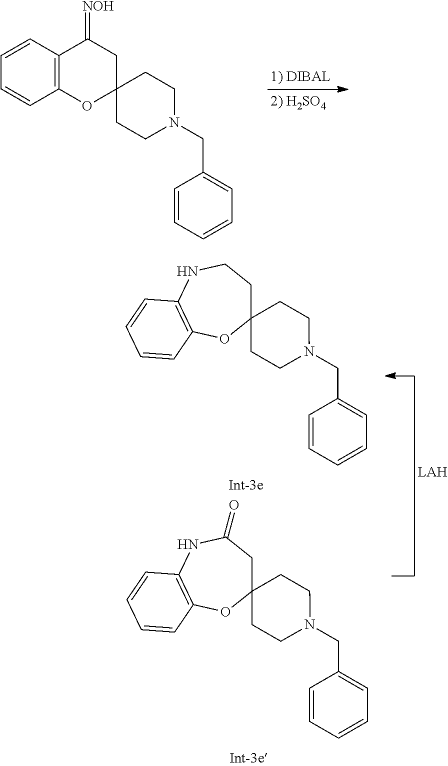

The oxime was synthesized according to the procedure published in Willand et al. (supra). The oxime (3.0 gm, 9.3 mmol) was dissolved in 20 ml of dry dichloromethane and stirred in an ice bath for 30 minutes. Diisobutylaluminium hydride in dichloromethane (1 N. 54 mL, 54 mmol, 5.8 equiv) was added dropwise over 1 h while stirring. The mixture was stirred for 3 h under argon at 0° C., and then quenched by slowly adding MeOH (9 mL) followed by distilled water (9 mL) and 20% sulfuric acid (50 mL). The solution was stirred for a further 20 min. After basified to pH9 using 30% sodium hydroxide solution, the reaction crude was extracted with ethyl acetate (100 mL twice). The organic layer was dried by MgSO₄ and concentrated to give a yellow oil. LCMS showed the major products are Int-3e′ and Int-3e (roughly 1:1). To the residue was added lithium aluminum hydride solution in tetrahydrofuran (1 N. 23.2 mL, 18.6 mmol, 2.5 eq) and refluxed for 2 hr. The reaction was cooled down to room temperature and quenched by slowly addition of ethyl acetate (100 mL) and saturated NaHCO₃ (50 mL). The white precipitate was filtered off. The filtrate was settled in a separation funnel. The organic layer was separated and washed by water, brine, dried over MgSO₄, and concentrated to afford a yellow oil. This crude product was used in the next reaction without further purification. LCMS: 309.2 (M+1) at 1.12 min (5 min method).

### Example 4

### Preparation of Intermediates of Type A4 Synthesis of tert-butyl 6-fluoro-4,5-dihydro-3H-spiro[benzo[b]-[1,4]oxazepine-2,4′-piperidine]-1′-carboxylate (Int-4-a)

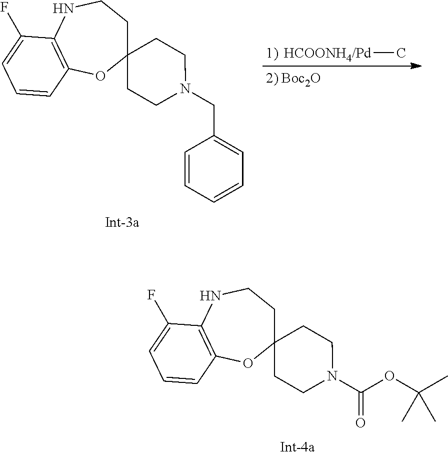

The crude Int-3a from Example 3 (0.50 gm) and ammonium formate (0.36 gm) were dissolved in methanol (6.6 mL). The mixture was flushed with argon followed by addition of palladium on activated carbon (10%, 0.36 gm). The reaction was stirred at 58° C. for 4 hours. After filtration through Celite, the filtrate was concentrated under reduced pressure.

The deprotected crude product (73 mg) was dissolved in a mixture of triethylamine (0.086 mL, 0.62 mmole), dichloromethane (0.52 mL) and dioxane (0.52 mL). A solution of di-tert-butyl dicarbonate (71 mg, 0.33 mmole) in dichloromethane (1.0 mL) was slowly added while stirring. The crude reaction mixture was stirred at room temperature overnight. The solvent was removed under vacuum. The residue was dissolved in dichloromethane, washed with 10% NaHCO₃ solution, brine, dried with Na₂SO₄, and loaded onto a silica column. The column was eluted with a linear gradient of hexane/ethyl acetate. The desired fractions were collected and the solvent was evaporated to afford the pure Int-4-a as a white solid. LCMS: 337.2 (M+1).

### Synthesis of tert-butyl 7-fluoro-4,5-dihydro-3H-spiro[benzo[b]-[1,4]oxazepine-2,4′-piperidine]-1′-carboxylate (Int-4-b)

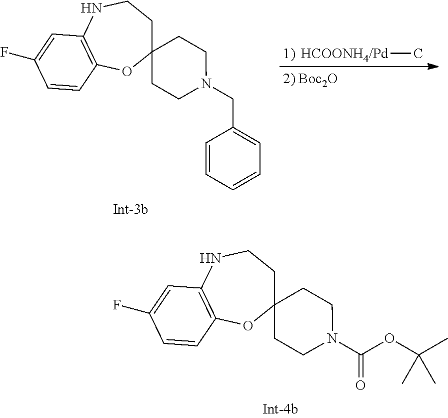

Int-4-b was synthesized in a similar fashion as Int-4-a using Int-3b as starting material. LCMS: 337.2 (M+1).

### Synthesis of tert-butyl 9-fluoro-4,5-dihydro-3H-spiro[benzo[b]-[1,4]oxazepine-2,4′-piperidine]-1′-carboxylate (Int-4-c)

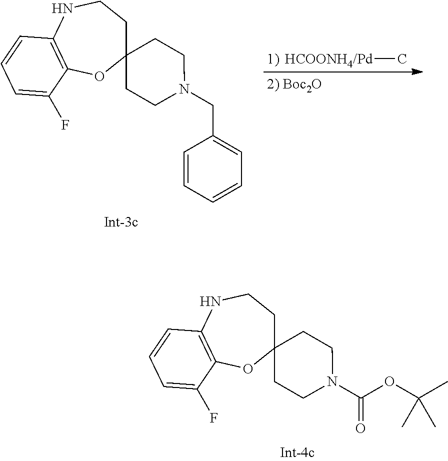

Int-4-c was synthesized in a similar fashion as Int-4-a using Int-3c as starting material. LCMS: 337.2 (M+1).

### Synthesis of tert-butyl 4,5-dihydro-3H-spiro[benzo[b][1,4]oxazepine-2,4′-piperidine]-1′-carboxylate (Int-4-e)

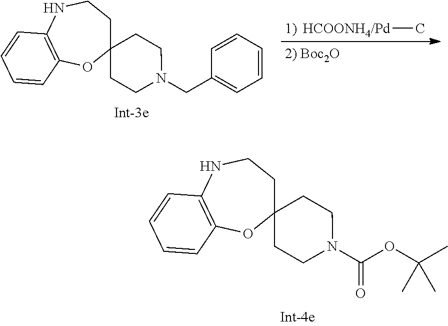

Int-4-e was synthesized in a similar fashion as Int-4-a using Int-3e as starting material. LCMS: 318.2 (M+1) at 1.38 min (5 min method).

### Example 5

### Preparation of Compound 1

This example illustrates the preparation of Compound of the Formula (I) wherein R² is —C(O)Y.

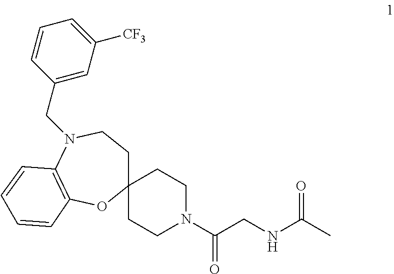

### Step A—Preparation of tert-Butyl 4,5-dihydro-3H-spiro[benzo[b][1,4]oxazepine-2,4′-piperidine]-1′-carboxylate (Int-5a)

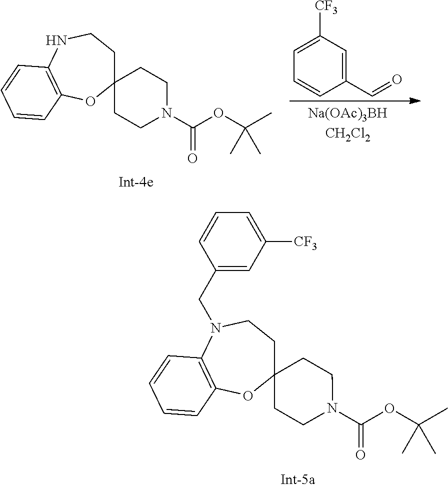

Int-4-e (300 mg, 0.94 mmole), 3-(trifluoromethyl)benzaldehyde (174 mg, 1.0 mmole), and triacetoxy sodium borohydride (414 mg, 2.0 mmole) were dissolved in dichloromethane (5.0 mL). The mixture was stirred at room temperature overnight, and then loaded onto a silica column. The column was eluted with a mixture of hexane/ethyl acetate, and the desired fractions were collected. The solvent was removed under vacuum to give the Int-5a as a white solid (408 mg, yield=91%). LCMS: 309.2 (M+1).

### Step B—Synthesis of 5-(3-(trifluoromethyl)benzyl)-4,5-dihydro-3H-spiro[benzo[b][1,4]oxazepine-2,4′-piperidine] (Int-5b)

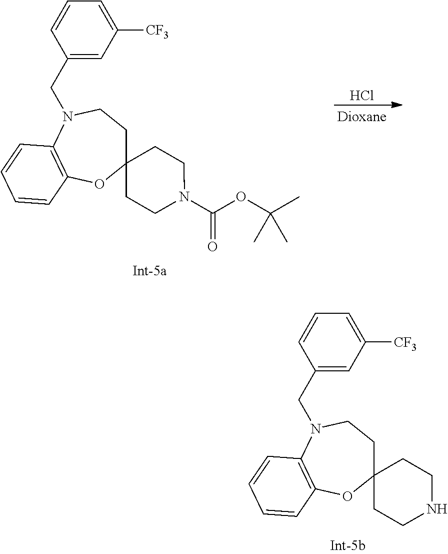

Int-5a (408 mg, 0.86 mmole) was dissolved in a THF solution of hydrochloric acid (4 N, 4 mL). The reaction was stirred at room temperature overnight. The solvent was removed in vacuo to afford the hydrochloric acid salt of Int-5b as a white solid (385 mg, quantitative).

### Step C—Synthesis of N-(2-oxo-2-(5-(3-(trifluoromethyl)benzyl)-4,5-dihydro-3H-spiro[benzo[b][1,4]oxazepine-2,4′-piperidine]-1′-yl)ethyl)acetamide (1)

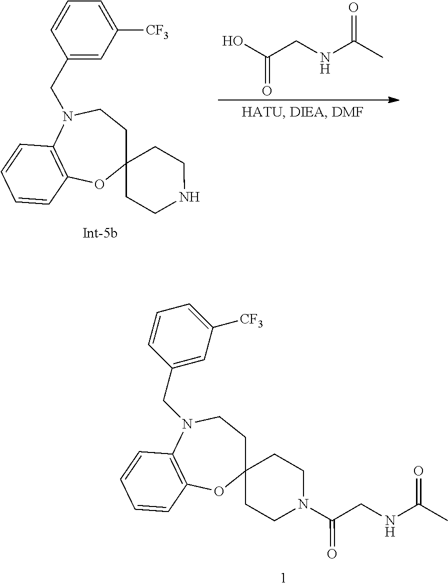

N-acetylglycine (26 mg, 0.22 mmole, 3.3 eq) and HATU (76 mg, 0.20 mmole, 3.0 eq) were dissolved in a mixture of DMF (2.0 mL) and DIEA (0.2 mL). Int-5b (30 mg, 0.067 mmole) was added to the mixture, and the reaction was stirred at room temperature overnight. The crude mixture was concentrated and the residue was purified by preparative LCMS. After lyophilization, the TEA salt of the product was dissolved in a mixture of HCl (1.0 N, 1.0 mL) and acetonitrile (1.0 mL). The solution was lyophilized again to afford hydrochloric acid salt of compound I. LCMS: 490.2 (M+1).

### Example 6

### Preparation of tert-butyl 6-chloro-4,5-dihydro-3H-spiro[benzo[b][1,4]oxazepine-2,4′-piperidine]-1′-carboxylate (Int-6a)

(Illustrates synthesis of intermediates useful in preparing Compounds of Formula (I) wherein R³ is 9-chloro)

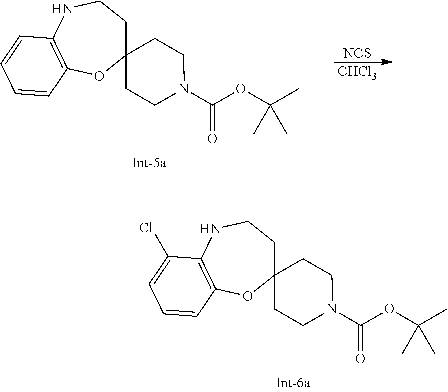

Int-5a (40 mg, 0.13 mmole) was dissolved in chloroform (0.25 mL). N-chlorosuccinimide (20 mg, 0.15 mmole, 1.1 eq) was added and the mixture was stirred at room temperature for 10 minutes. The crude product was loaded onto a silica column, and eluted with a liner gradient of hexane-ethyl acetate. The desired fractions were collected and the solvent was evaporated to afford the product, Int-6a. LCMS: 352.2 (M+1). Int-6a can be used to prepare compounds wherein R³ is 9-chloro.

Using the methods described in Examples 1-6, compounds of the formula (Ia) are prepared.

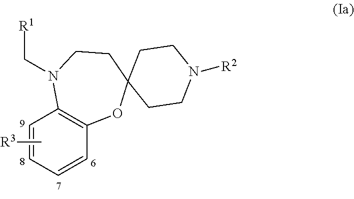

Representative compounds 1-49 in Table 1 (see below) were prepared using these methods.

### Example 7

### Preparation of methyl 2-(5-(3-(trifluoromethyl)benzyl)-4,5-dihydro-3H-spiro[benzo[b][1,4]oxazepine-2,4′-piperidine]-1′-yl)thiazole-5-carboxylate](Compound 50)

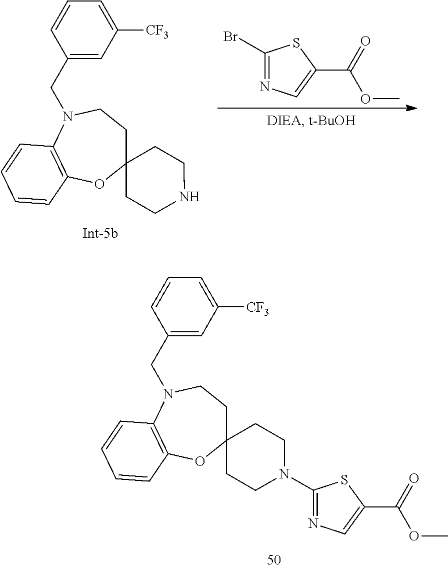

5-(3-(trifluoromethyl)benzyl)-4,5-dihydro-3H-spiro[benzo[b][1,4]oxazepine-2,4′-piperidine]dihydrochloric acid salt (1.8 gm, 4.0 mmole) and methyl 2-bromothiazole-5-carboxylate (1.0 gm, 4.5 mmole, 1.12 eq) were dissolved in a mixture of tert-butanol (10.0 mL) and diisopropylethylamine (2.0 mL). The mixture was heated to 165° C. for 10 min in a Biotage microwave synthesizer. The solvent was removed under vacuum. The residue was dissolved in minimum amount of dichloromethane and loaded onto a silica column, eluted with a gradient of hexane and ethyl acetate. The desired fractions were collected and concentrated to give compound 50 as a white solid (1.92 gm, yield=93%)

### Example 8

### Preparation of 2-(5-(3-(trifluoromethyl)benzyl)-4,5-dihydro-3H-spiro[benzo[b][1,4]oxazepine-2,4′-piperidine]-1′-yl)thiazole-5-carboxylic Acid (Compound 54)

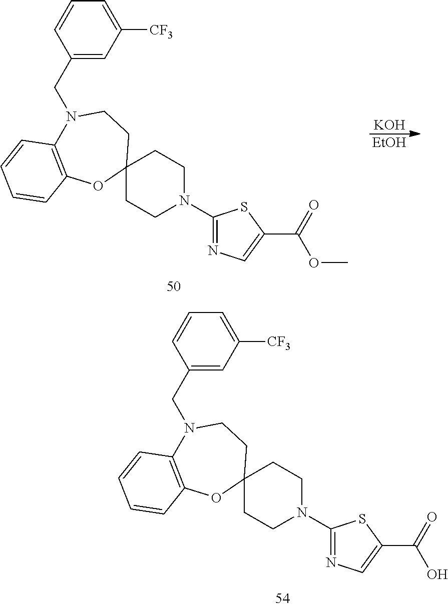

Potassium hydroxide (1.0 gm, 17.9 mmole) was dissolved in ethanol (30 mL, 9.0 eq) and was added to compound 50 (1.0 gm). The mixture was refluxed and monitored by TLC. When all the starting material disappeared, the reaction was neutralized with HCl in dioxane (4.0 M. 4.48 mL, 17.9 mmole). The solid was filtered off, and the filtrate was concentrated to give the compound 54 as a slightly off-white solid (0.866 gm, yield=89%).

### Example 9

### Preparation of N-methyl-2-(5-(3-(trifluoromethyl)benzyl)-4,5-dihydro-3H-spiro[benzo[b][1,4]oxazepine-2,4′-piperidine]-1′-yl)thiazole-5-carboxamide (58)

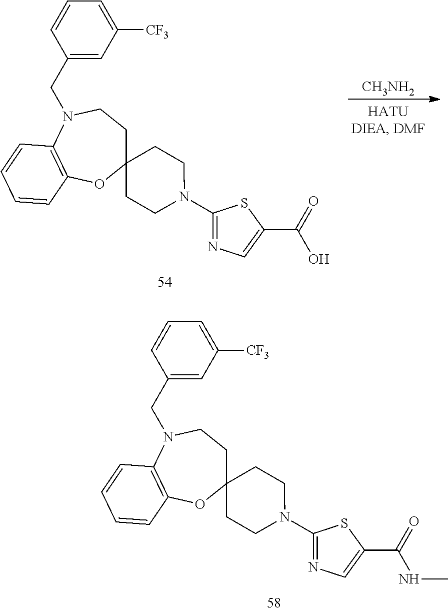

Compound 54 (50 mg, 0.10 mmole) and HATU (38 mg, 0.10 mmole, 1.0 eq) were dissolved in a mixture of dimethylformamide (0.50 mL) and diisopropylethylamine (0.10 mL). Methylamine in THF (2.0 N. 0.20 mL, 4 eq) was added and the reaction was stirred at room temperature overnight. The solvent was removed under vacuum, and the residue was purified by preparative LC-MS. The desired fractions were collected and lyophilized to give compound 58 as a white powder (42 mg, yield=82%).

Following procedures similar to those described in Examples 7-9, compounds of the Formula (Ib) can be prepared

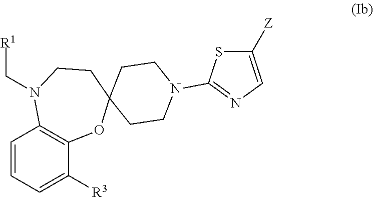

For instance, compounds 50-138 in Table 2 (belo ere prepared using such procedures.

### Example 10

### Preparation of methyl 6-(5-(3-(trifluoromethyl)benzyl)-4,5-dihydro-3H-spiro-[benzo[b][1,4]oxazepine-2,4′-piperidine]-1′-yl)pyridazine-3-carboxylate (Compound 170)

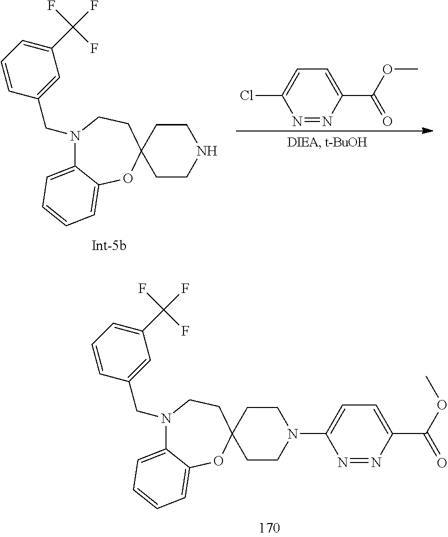

The hydrochloric acid salt of Int-5b (450 mg, 1.0 mmole) and methyl 6-chloropyridazine-3-carboxylate (190 mg, 1.1 mmole, 1.1 eq) were dissolved in a mixture of t-butyl alcohol (5.0 mL) and diisopropylethylamine (1.0 mL). The mixture was heated to 170° C. for 15 min in a Biotage microwave synthesizer.

The solvent was removed under vacuum, and the residue was purified with a silica column. Compound 170 was obtained as a white solid (480 mg, yield=94%).

### Example 11

### Preparation of 6-(5-(3-(trifluoromethyl)benzyl)-4,5-dihydro-3H-spiro[benzo-[b][1,4]oxazepine-2,4′-piperidine]-1′-yl)pyridazine-3-carboxylic Acid (Compound 172)

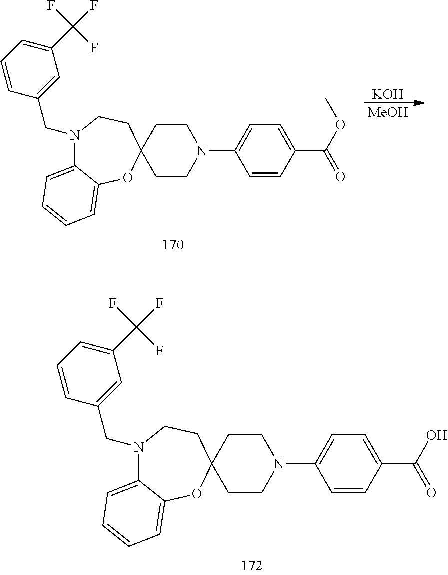

Compound 170 (400 mg, 0.78 mmole) and potassium hydroxide (400 mg, 7.1 mmole, 9 eq) was dissolved in methanol (5 mL). The mixture was stirred at 50° C. overnight. Hydrochloric acid in dioxane (4.0M, 1.78 mL) was added, and the precipitation was filtered off. The filtrate was concentrated under vacuum to give compound 172 as a white solid (346 mg, yield=89%).

### Example 12

### Preparation of 6-(5-(3-(trifluoromethyl)-benzyl)-4,5-dihydro-3H-spiro-[benzo[b]-[1,4]oxazepine-2,4′-piperidine]-1′-yl)pyridazine-3-carboxylic Acid (Compound 155)

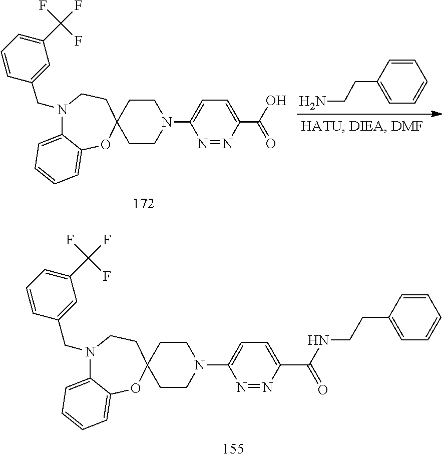

Compound 172 (50 mg, 0.10 mmole) and HATU (38 mg, 1.0 mmole, 1.0 eq) were dissolved in a mixture of dimethylformamide (1.0 mL) and diisopropylethylamine (0.10 mL). 2-Phenylethanamine (18.2 mg, 0.15 mmole, 1.5 eq) was added and the mixture was stirred at room temperature overnight. Solvent was removed under high vacuum, and the residue was purified by preparative LC-MS. The desired fractions were collected and lyophilized. The product was dissolved in a 1:1 mixture of acetonitrile and 1N HCl (2.0 mL) and was lyophilized again. Compound 155 was obtained as a white solid (38.0 mg, yield=63%).

Following procedures similar to those described in Examples 10-12, compounds of the Formula (Ic) can be prepared.

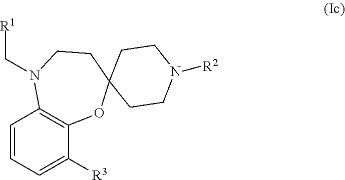

For instance, compounds 139-184 in Table 3 were prepared using such methods.

### Example 13

### Stearoyl-CoA desaturase Assay

Stearoyl-CoA desaturase assays were performed in according to Talamo and Bloch. See Talamo, B R & Bloch, K, A new assay for fatty acid desaturation,” 29 Anal. Biochem. 300-304 (1969). Assays were run in triplicate in 100-μl volumes of 100 mM TrisHCl, pH 7.3, containing 20 μM stearoyl-CoA, 2 mM β-NADH, and 50 μg of protein from a HepG2 cell P2 pellet. Since SCD-1 is the only isoform of SCD expressed in these cells, the assay is specific for SCD-1 with these cells as the source of enzyme. Reaction mixtures were incubated fifteen minutes at 25° C. and reactions were then stopped with a volume of trichloroacetic acid giving a final concentration of 0.2%. After five minutes, a 90-μl volume was transferred to a Millipore Multiscreen HTS 96-well filtration plate (MSHVN4B50) containing 125 μl of 10% charcoal in each well, to which vacuum had been previously applied. Plates were shaken fifteen minutes and then filtered into a collection plate. Fifty-μI volumes of filtrate were transferred to another plate containing 150 μl of MicroScint 40 for counting on a TopCount scintillation counter. Total activity was determined in reaction mixtures containing 2% DMSO and blank with a standard inhibitor at 10⁻⁴ M. Test compounds were run at five concentrations from 10⁻⁵ to 10⁻⁹ M and IC₅₀ values were interpolated from the data.

Stearoyl CoA desaturase assay data was determined for certain compounds of the present invention using the above-described method. IC₅₀ data for selected compounds of the present invention, i.e., compounds I-49 (Table 1), compounds 50-138 (Table 2), and compounds 139-184 (Table 3), are provided below wherein A is 0.5-49 nM, B is 50-499 nM, C is 500-10,000 nM, and D is >10,000 nM.

TABLE 1

| | |
|---|---|
| | (Ia) |
| 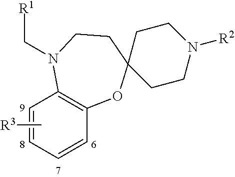 | |

| | | | | | LCMass Spec |
|---|---|---|---|---|---|
| | | | | Ave IC₅₀ | M + 1 @ ret. |
| Compound | R¹ | R² | R³ | nM | time |
| 1 | 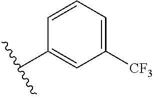 | 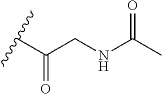 | H | D | 490.2 @ 4.39 min |
| 2 | 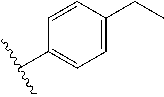 | 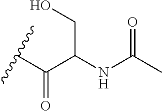 | H | D | 466.3 @ 3.33 min |
| 3 | 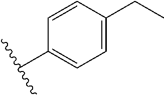 | 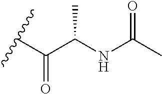 | H | D | 450.3 @ 3.68 min |
| 4 | 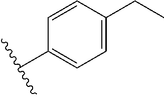 | 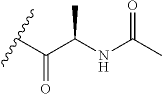 | H | C | 450.3 @ 3.67 min |
| 5 | 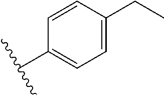 | 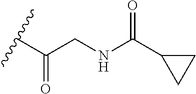 | H | C | 462.3 @ 3.68 min |
| 6 | 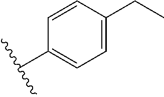 | 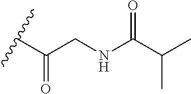 | H | C | 464.3 @ 3.76 min |
| 7 | 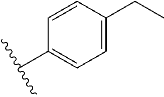 | 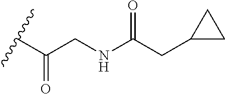 | H | B | 476.3 @ 3.84 min |
| 8 | 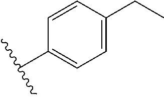 | 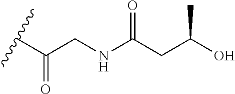 | H | C | 480.3 @ 3.44 min |
| 9 | 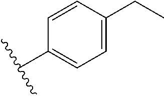 | 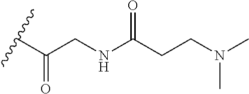 | H | D | 493.3 @ 3.36 min |
| 10 | 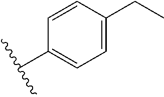 | 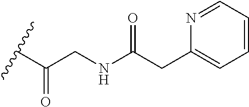 | H | D | 513.3 @ 3.42 min |
| 11 | 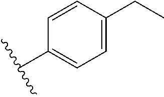 | 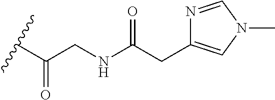 | H | C | 516.3 @ 3.35 min |
| 12 | 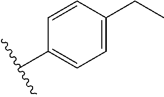 | 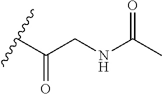 | 9-F | B | 454.2 @ 4.28 min |
| 13 | 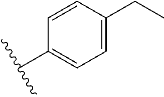 | 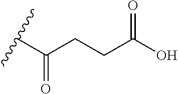 | H | C | 494.3 @ 3.33 min |
| 14 | 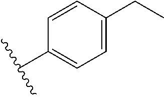 | 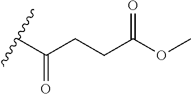 | H | C | 508.3 @ 3.61 min |
| 15 | 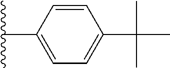 | 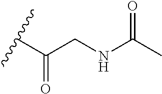 | 6-Cl | C | 498.2 @ 4.94 min |
| 16 | 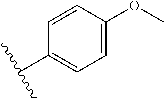 | 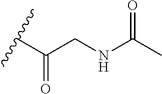 | H | C | 438.2 @ 2.67 min |
| 17 | 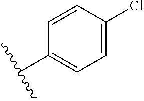 | 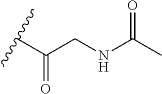 | H | D | 442.2 @ 3.56 min |
| 18 | 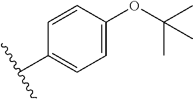 | 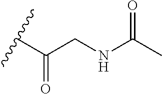 | H | D | 480.3 @ 3.42 min |
| 19 | 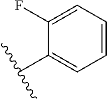 | 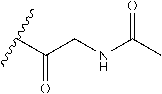 | H | D | 426.2 @ 3.22 min |
| 20 | 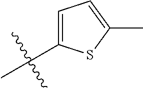 | 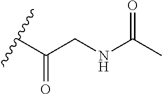 | H | D | 428.2 @ 3.20 min |
| 21 | 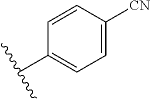 | 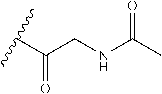 | H | C | 433.2 @ 3.20 min |
| 22 | 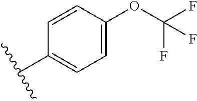 | 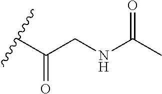 | H | C | 492.2 @ 3.95 min |
| 23 | 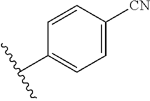 | 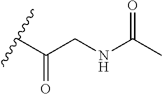 | H | D | 433.2 @ 3.25 min |
| 24 | 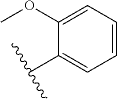 | 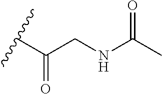 | H | C | 438.2 @ 2.56 min |
| 25 | 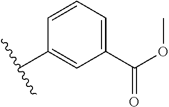 | 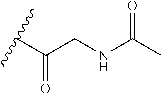 | H | C | 466.2 @ 3.27 min |
| 26 | 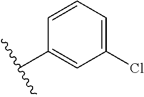 | 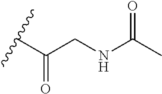 | H | C | 442.2 @ 3.68 min |
| 27 | 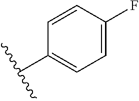 | 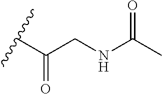 | H | B | 426.2 @ 3.04 min |
| 28 | 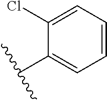 | 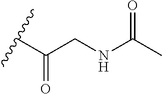 | H | C | 442.2 @ 3.81 min |
| 29 | 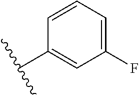 | 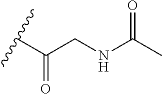 | H | C | 426.2 @ 3.34 min |
| 30 | 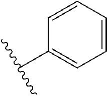 | 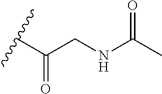 | H | C | 408.2 @ 2.92 min |
| 31 | 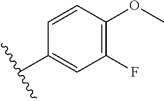 | 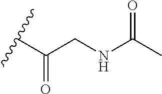 | H | C | 456.2 @ 3.09 min |
| 32 | 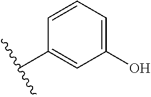 | 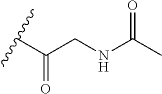 | H | C | 424.2 @ 2.34 min |
| 33 | 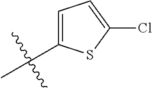 | 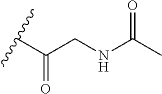 | H | C | 448.1 @ 3.81 min |
| 34 | 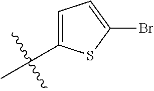 | 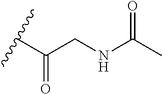 | H | C | 492.1 @ 3.84 min |
| 35 | 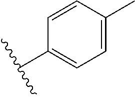 | 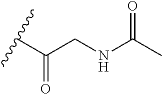 | H | C | 422.2 @ 3.16 min |
| 36 | 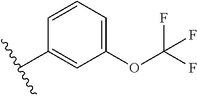 | 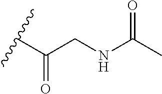 | H | C | 492.2 @ 4.54 min |
| 37 | 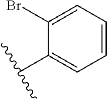 | 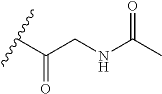 | H | C | 486.1 @ 4.00 min |
| 38 | 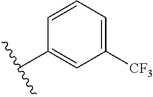 | 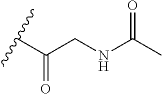 | 7-F | C | 494.2 @ 4.17 min |
| 39 | 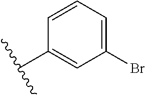 | 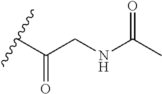 | H | C | 486.1 @ 3.83 min |
| 40 | 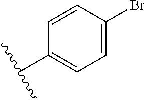 | 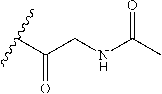 | H | C | 486.1 @ 3.77 min |
| 41 | 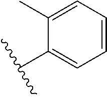 | 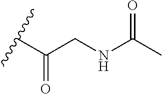 | H | C | 422.2 @ 3.46 min |
| 42 | 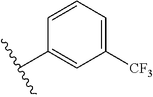 | 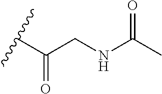 | 9-Cl | C | 510.2 @ 5.73 min |
| 43 | 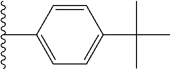 | 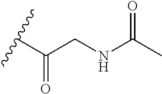 | 9-Cl | C | 498.2 @ 6.44 min |
| 44 | 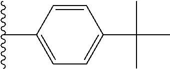 | 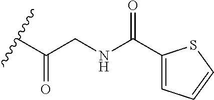 | H | B | 532.3 @ 6.61 min |
| 45 | 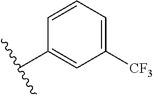 | 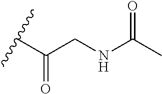 | 6-F | C | 494.2 @ 5.47 min |
| 46 | 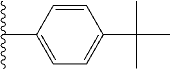 | 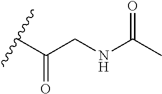 | 6-F | C | 482.3 @ 6.19 min |
| 47 | 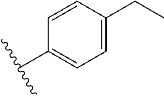 | 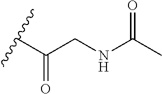 | 6-F | C | 454.2 @ 5.70 min |
| 48 | 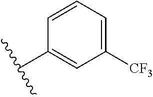 | 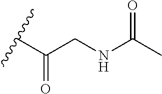 | 9-F | C | 494.2 @ 5.47 min |
| 49 | 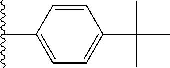 | 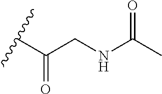 | 9-F | B | 482.3 @ 6.21 min |

TABLE 2

| | |
|---|---|
| | (Ib) |
| 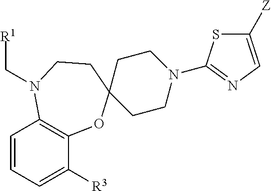 | |

| | | | | | LCMass Spec |
|---|---|---|---|---|---|
| | | | | Ave IC₅₀ | M + 1 @ ret. |
| Compound | R1 | Z | R³ | nM | time |
| 50 | 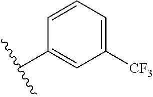 | 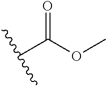 | H | A | 518.2 @ 7.14 min |
| 51 | 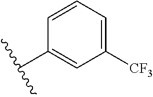 | 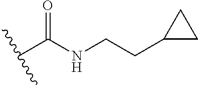 | H | C | 571.2 @ 6.61 min |
| 52 | 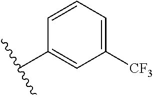 | 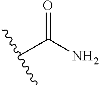 | H | A | 503.2 @ 5.67 min |
| 53 | 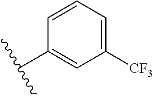 | 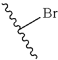 | H | C | 538.1 @ 7.61 min |
| 54 | 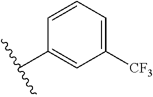 | 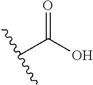 | H | A | 504.1 @ 6.06 min |
| 55 | 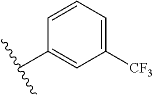 | 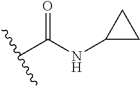 | H | C | 546.2 @ 6.05 min |
| 56 | 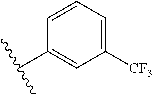 | 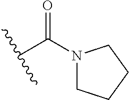 | H | C | 557.2 @ 6.23 min |
| 57 | 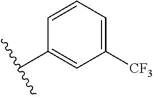 | 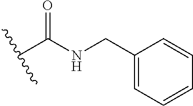 | H | C | 593.2 @ 6.55 min |
| 58 | 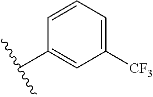 | 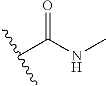 | H | A | 517.2 @ 5.77 min |
| 59 | 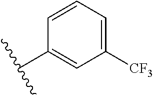 | 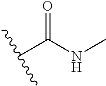 | H | A | 535.2 @ 5.81 min |
| 60 | 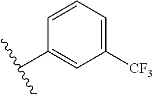 | 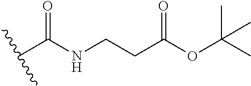 | H | B | 631.2 @ 6.62 min |
| 61 | 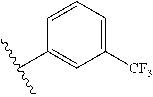 | 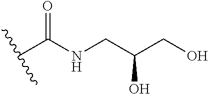 | H | A | 577.2 @ 5.29 min |
| 62 | 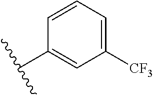 | 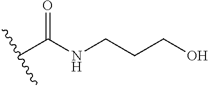 | H | A | 561.2 @ 5.57 min |
| 63 | 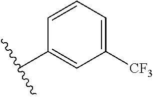 | 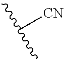 | H | A | 485.2 @ 2.53 min |
| 64 | 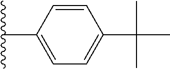 | 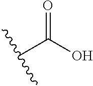 | H | B | 492.2 @ 6.52 min |
| 65 | 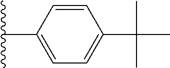 | 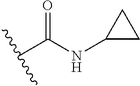 | H | B | 531.3 @ 6.52 min |
| 66 | 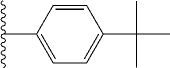 | 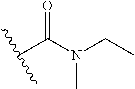 | H | B | 533.3 @ 6.79 min |
| 67 | 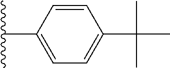 | 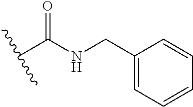 | H | C | 581.3 @ 7.01 min |
| 68 | 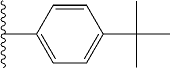 | 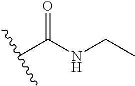 | H | A | 519.3 @ 6.49 min |
| 69 | 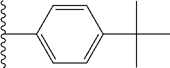 | 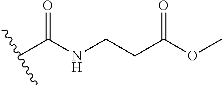 | H | A | 577.3 @ 6.47 min |
| 70 | 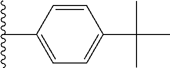 | 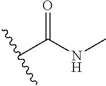 | H | A | 505.3 @ 6.26 min |
| 71 | 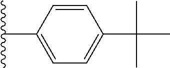 | 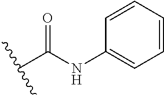 | H | D | 567.3 @ 7.19 min |
| 72 | 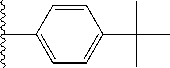 | 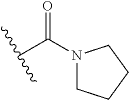 | H | C | 545.3 @ 6.75 min |
| 73 | 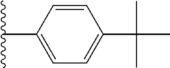 | 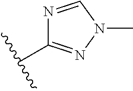 | H | A | 529.3 @ 6.29 min |
| 74 | 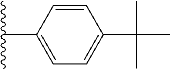 | 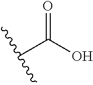 | F | A | 510.2 @ 2.58 min |
| 75 | 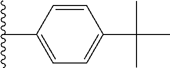 | 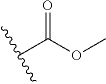 | F | A | 536.2 @ 2.56 min |
| 76 | 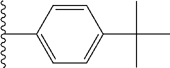 | 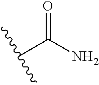 | F | A | 509.2 @ 6.33 min |
| 77 | 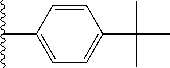 | 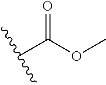 | F | A | 595.3 @ 6.69 min |
| 78 | 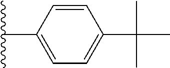 | 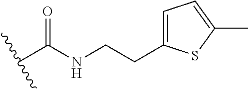 | F | C | 633.3 @ 5.84 min |
| 79 | 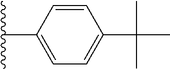 | 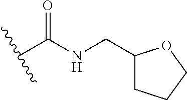 | F | B | 593.3 @ 5.40 min |
| 80 | 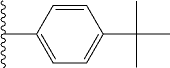 | 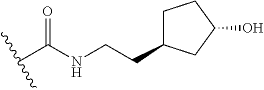 | F | C | 621.3 @ 5.27 min |
| 81 | 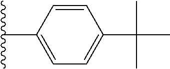 | 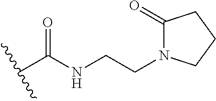 | F | C | 620.3 @ 5.26 min |
| 82 | 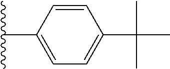 | 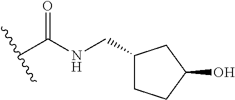 | F | D | 607.3 @ 5.17 min |
| 83 | 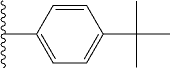 | 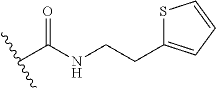 | F | C | 619.3 @ 5.67 min |
| 84 | 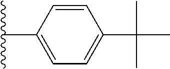 | 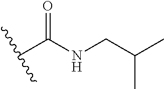 | F | C | 565.3 @ 5.58 min |
| 85 | 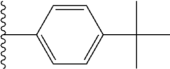 | 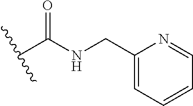 | F | B | 600.3 @ 4.98 min |
| 86 | 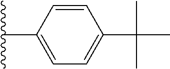 | 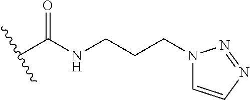 | F | C | 618.3 @ 5.17 min |
| 87 | 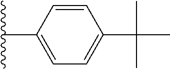 | 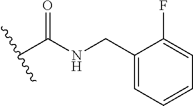 | F | B | 617.3 @ 5.68 min |
| 88 | 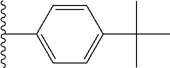 | 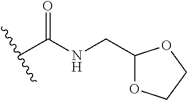 | F | A | 595.3 @ 5.25 min |
| 89 | 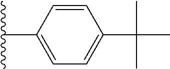 | 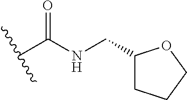 | F | B | 593.3 @ 5.40 min |
| 90 | 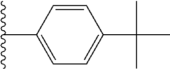 | 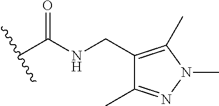 | F | C | 631.3 @ 5.16 min |
| 91 | 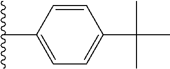 | 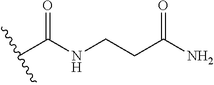 | F | B | 580.3 @ 4.97 min |
| 92 | 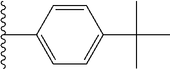 | 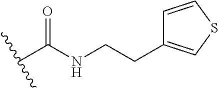 | F | C | 619.2 @ 5.67 min |
| 93 | 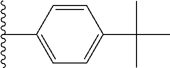 | 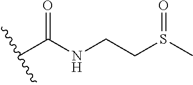 | F | B | 599.2 @ 5.18 min |
| 94 | 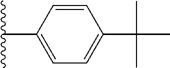 | 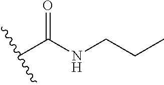 | F | B | 551.3 @ 5.43 min |
| 95 | 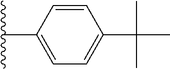 | 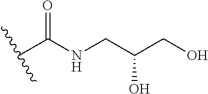 | F | B | 583.3 @ 4.90 min |
| 96 | 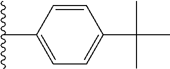 | 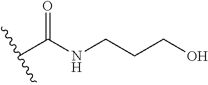 | F | A | 567.3 @ 5.06 min |
| 97 | 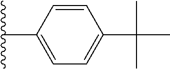 | 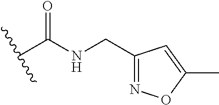 | F | C | 604.3 @ 5.38 min |
| 98 | 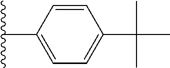 | 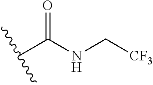 | F | B | 591.2 @ 5056 min |
| 99 | 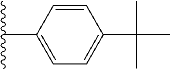 | 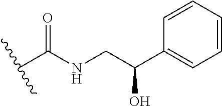 | F | C | 629.3 @ 5.37 min |
| 100 | 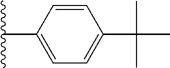 | 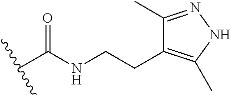 | F | D | 631.3 @ 5.73 min |
| 101 | 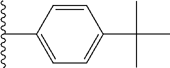 | 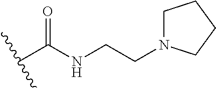 | F | C | 606.3 @ 5.66 min |
| 102 | 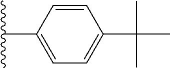 | 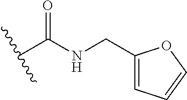 | F | C | 589.2 @ 6.94 min |
| 103 | 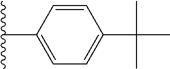 | 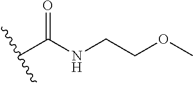 | F | B | 567.3 @ 6.64 min |
| 104 | 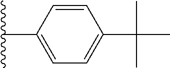 | 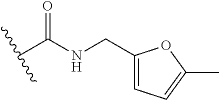 | F | B | 603.3 @ 7.11 min |
| 105 | 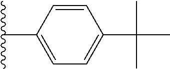 | 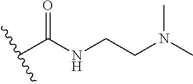 | F | D | 580.3 @ 5.57 min |
| 106 | 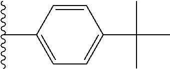 | 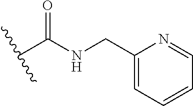 | F | D | 600.3 @ 5.60 min |
| 107 | 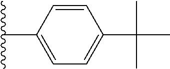 | 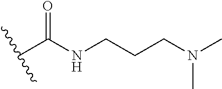 | F | C | 594.3 @ 5.57 min |
| 108 | 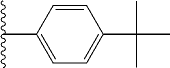 | 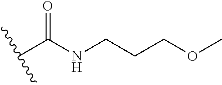 | F | B | 581.3 @ 6.75 min |
| 109 | 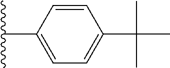 | 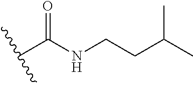 | F | C | 579.3 @ 7.28 min |
| 110 | 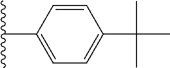 | 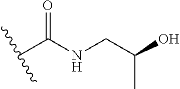 | F | A | 567.3 @ 6.34 min |
| 111 | 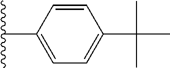 | 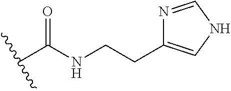 | F | D | 603.3 @ 5.47 min |
| 112 | 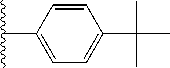 | 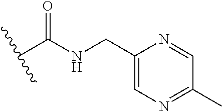 | F | C | 615.3 @ 6.65 min |
| 113 | 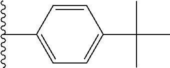 | 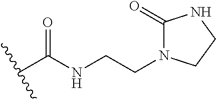 | F | C | 621.3 @ 6.15 min |
| 114 | 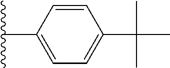 | 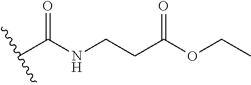 | F | C | 623.3 @ 6.84 min |
| 115 | 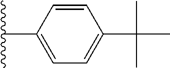 | 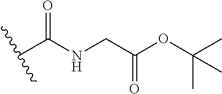 | F | A | 623.3 @ 7.18 min |
| 116 | 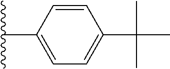 | 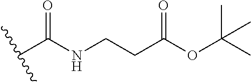 | F | A | 637.3 @ 7.24 min |
| 117 | 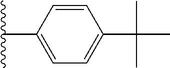 | 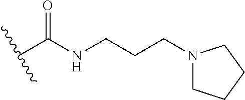 | F | D | 620.3 @ 5.59 min |
| 118 | 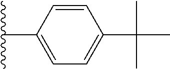 | 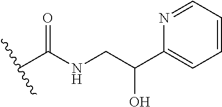 | F | B | 630.3 @ 5.53 min |
| 119 | 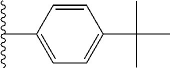 | 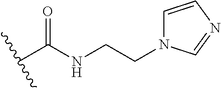 | F | D | 603.3 @ 5.44 min |
| 120 | 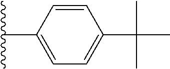 | 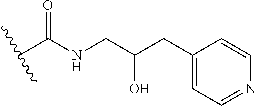 | F | C | 630.3 @ 5.38 min |
| 121 | 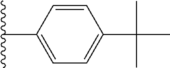 | 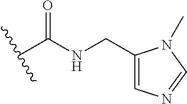 | F | D | 603.3 @ 5.51 min |
| 122 | 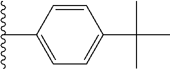 | 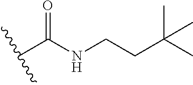 | F | D | 593.3 @ 7.42 min |
| 123 | 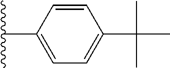 | 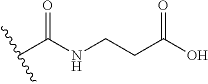 | F | A | 581.3 @ 6.21 min |
| 124 | 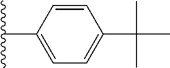 | 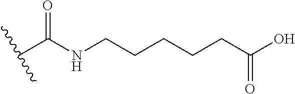 | F | C | 623.3 @ 6.41 min |
| 125 | 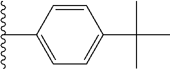 | 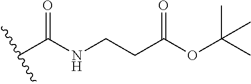 | H | C | 619.3 @ 7.12 min |
| 126 | 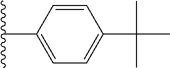 | 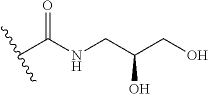 | H | A | 565.3 @ 5.81 min |
| 127 | 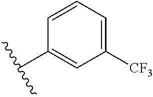 | 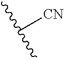 | H | A | 485.2 @ 5.3 min |
| 128 | 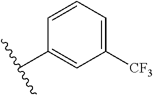 | 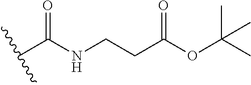 | H | B | 649.1 @ 6.7 min |
| 129 | 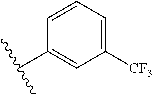 | 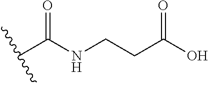 | H | A | 575.1 @ 5.5 min |
| 130 | 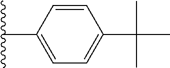 | 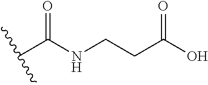 | H | A | 563.2 @ 4.7 min |
| 131 | 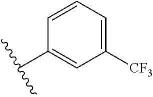 | 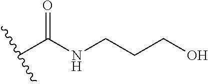 | F | A | 579.2 @ 5.7 min |
| 132 | 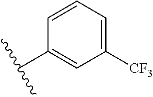 | 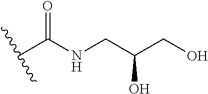 | F | A | 595.1 @ 5.42 min |
| 133 | 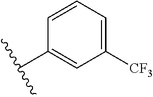 | 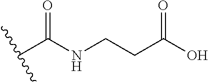 | F | A | 593.2 @ 5.8 min |
| 134 | 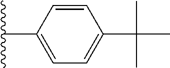 | 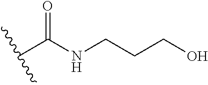 | H | A | 549.2 @ 6.0 min |
| 135 | 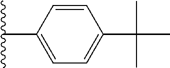 | 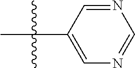 | H | B | 526.2 @ 5.7 min |
| 136 | 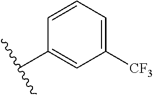 | 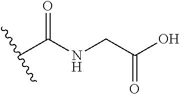 | H | A | 561.1 @ 5.6 min |
| 137 | 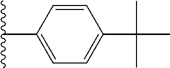 | 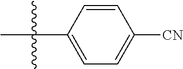 | H | C | 549.2 @ 6.01 min |
| 138 | 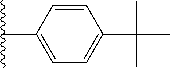 | 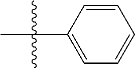 | H | C | 524 @ 7.3 min |

TABLE 3

| | |
|---|---|
| | (Ic) |
| 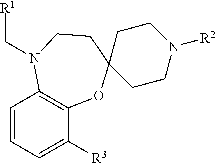 | |

| | | | | | LCMass |
|---|---|---|---|---|---|
| | | | | | Spec M + 1 @ |
| | | | | Ave | ret. |
| Compound | R¹ | R² | R³ | IC₅₀ nM | time |
| 139 | 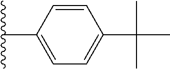 | 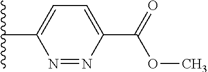 | H | C | 501.3 @ 4.47 min |
| 140 | 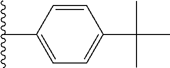 | 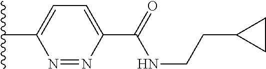 | H | A | 554.3 @ 7.04 min |
| 141 | 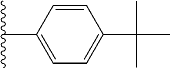 | 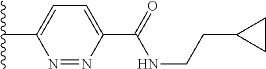 | H | B | 553.3 @ 5.84 min |
| 142 | 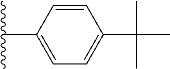 | 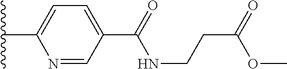 | F | B | 590.3 @ 6.59 min |
| 143 | 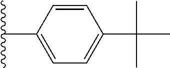 | 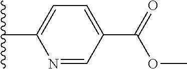 | F | B | 518.3 @ 5.40 min |
| 144 | 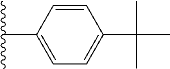 | 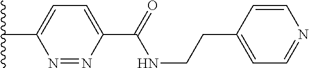 | F | A | 609.3 @ 5.43 min |
| 145 | 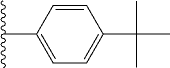 | 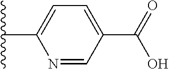 | F | B | 504.3 @ 5.91 min |
| 146 | 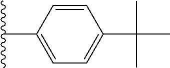 | 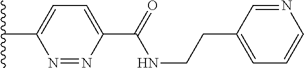 | F | B | 608.3 @ 4.97 min |
| 147 | 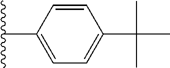 | 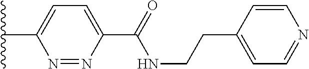 | F | B | 608.3 @ 4.96 min |
| 148 | 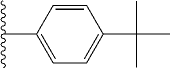 | 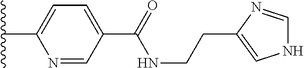 | F | C | 597.3 @ 4.91 min |
| 149 | 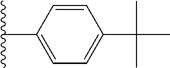 | 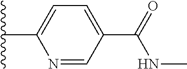 | F | A | 503.3 @ 5.47 min |
| 150 | 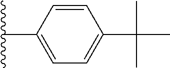 | 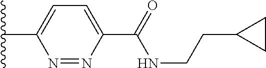 | F | B | 572.3 @ 7.07 min |
| 151 | 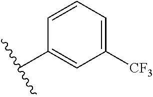 | 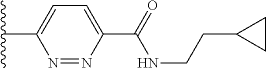 | H | A | 566.3 @ 6.54 min |
| 152 | 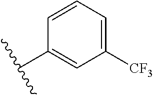 | 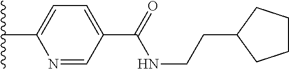 | H | C | 595.3 @ 4.81 min |
| 153 | 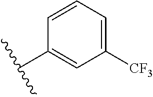 | 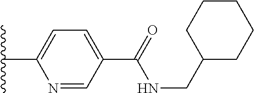 | H | C | 594.3 @ 7.14 min |
| 154 | 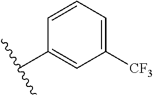 | 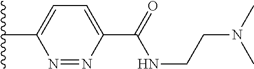 | H | C | 569.3 @ 4.69 min |
| 155 | 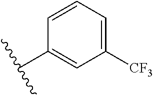 | 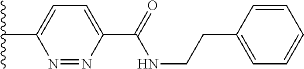 | H | A | 602.3 @ 6.71 min |
| 156 | 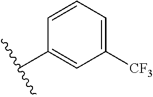 | 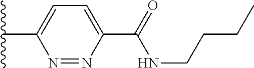 | H | A | 554.3 @ 6.55 min |
| 157 | 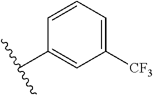 | 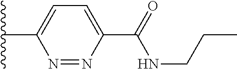 | H | B | 540.3 @ 6.26 min |
| 158 | 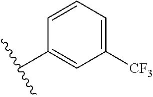 | 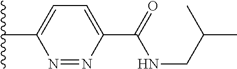 | H | B | 554.3 @ 6.56 min |
| 159 | 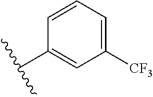 | 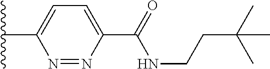 | H | C | 582.3 @ 6.99 min |
| 160 | 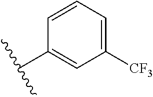 | 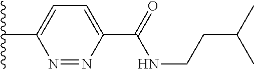 | H | B | 568.3 @ 6.80 min |
| 161 | 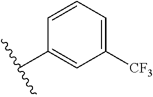 | 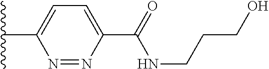 | H | B | 556.2 @ 5.35 min |
| 162 | 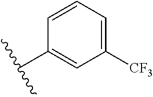 | 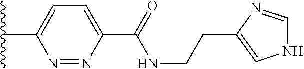 | H | A | 592.3 @ 4.68 min |
| 163 | 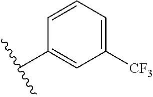 | 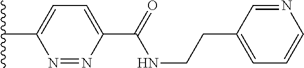 | H | A | 603.3 @ 4.74 min |
| 164 | 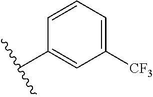 | 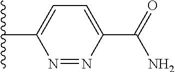 | H | A | 498.2 @ 5.43 min |
| 165 | 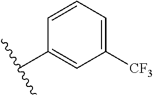 | 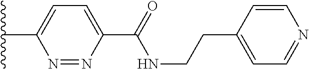 | H | A | 603.3 @ 4.77 min |
| 166 | 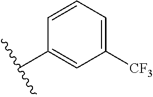 | 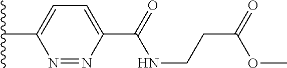 | H | A | 584.2 @ 5.93 min |
| 167 | 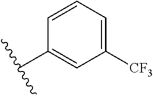 | 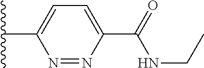 | H | B | 526.2 @ 5.96 min |
| 168 | 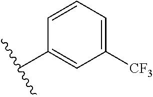 | 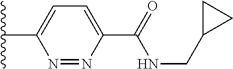 | H | B | 552.3 @ 6.33 min |
| 169 | 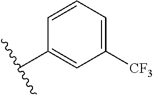 | 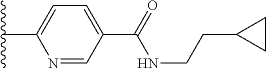 | H | A | 565.3 @ 5.47 min |
| 170 | 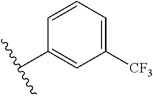 | 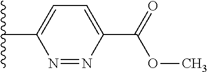 | H | A | 513.2 @ 6.13 min |
| 171 | 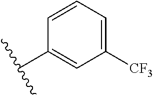 | 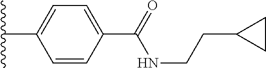 | H | C | 564.3 @ 6.83 min |
| 172 | 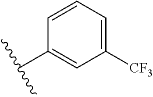 | 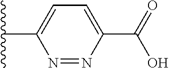 | H | C | 499.2 @ 5.30 min |
| 173 | 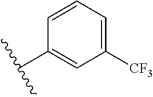 | 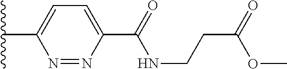 | F | A | 602.2 @ 5.92 min |
| 174 | 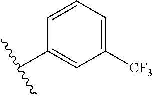 | 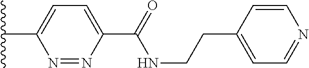 | F | A | 621.3 @ 4.80 min |
| 175 | 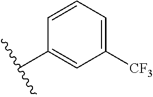 | 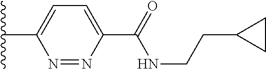 | F | A | 584.3 @ 6.52 min |
| 176 | 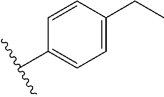 | 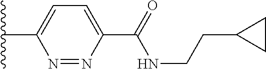 | H | A | 526.3 @ 6.57 min |
| 177 | 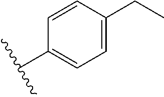 | 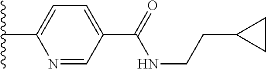 | H | B | 525.3 @ 5.46 min |
| 178 | 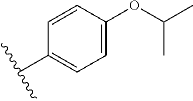 | 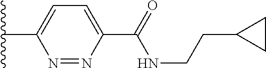 | H | A | 556.3 @ 6.20 min |
| 179 | 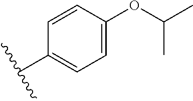 | 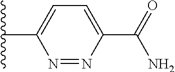 | H | A | 488.3 @ 4.73 min |
| 180 | 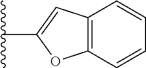 | 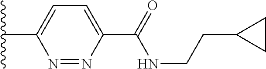 | H | A | 538.3 @ 6.29 min |
| 181 | 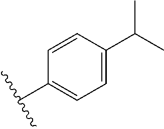 | 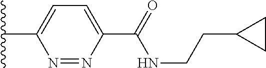 | H | A | 540.3 @ 6.78 min |
| 182 | 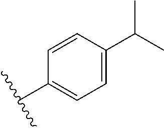 | 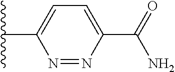 | H | A | 472.3 @ 5.47 min |
| 183 | 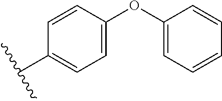 | 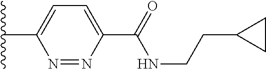 | H | A | 590.3 @ 6.68 min |
| 184 | 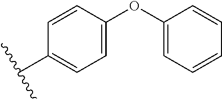 | 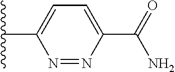 | H | A | 522.2 @ 5.49 min |

### Uses of the Benzo-Fused Oxazepine Compounds

The Benzo-Fused Oxazepine Compounds are useful in human and veterinary medicine. The Benzo-Fused Oxazepine Compounds are useful in a method of inhibiting the stearoyl-coenzyme A delta-9 desaturase enzyme (SCD) in a patient such as a mammal in need of such inhibition comprising the administration of an effective amount of the compound. The Benzo-Fused Oxazepine Compounds are therefore useful to control, prevent, and/or treat conditions and diseases mediated by high or abnormal SCD enzyme activity. For instance, the Benzo-Fused Oxazepine Compounds can be administered to a patient in need of treatment for a metabolic or skin disease/disorder.

The general value of the compounds of the invention in inhibiting, the activity of SCD can be determined, for example, using the assay described above in Example 13. Alternatively, the general value of the compounds in treating disorders and diseases may be established in industry standard animal models for demonstrating the efficacy of compounds in treating, for example, acne, obesity, diabetes or elevated triglyceride or cholesterol levels or for improving glucose tolerance. For instance, for testing whether the compounds are capable of modulating sebaceous gland function and sebum secretion, the assay described in Luderschmidt et al., Effects of cyproterone acetate and carboxylic acid derivatives on the sebaceous glands of the Syrian hamster, 258(2) Arch Dermatol Res. 185-91 (1977).

In one embodiment, the present invention provides a method of treating hyperglycemia, diabetes or insulin resistance in a mammalian patient in need of such treatment, which comprises administering to said patient an effective amount of a Benzo-Fused Oxazepine Compound or a pharmaceutically salt thereof.

In another embodiment, the present invention provides a method of treating non-insulin dependent diabetes mellitus (Type 2 diabetes) in a mammalian patient in need of such treatment comprising administering to the patient an antidiabetic effective amount of a Benzo-Fused Oxazepine Compound.

In another embodiment, the present invention provides a method of treating obesity in a mammalian patient in need of such treatment comprising administering to said patient a Benzo-Fused Oxazepine Compound in an amount that is effective to treat obesity.

In yet another embodiment, the present invention provides a method of treating metabolic syndrome and its sequelae in a mammalian patient in need of such treatment comprising administering to said patient a Benzo-Fused Oxazepine Compound in an amount that is effective to treat metabolic syndrome and its sequelae. The sequelae of the metabolic syndrome include hypertension, elevated blood glucose levels, high triglycerides, and low levels of HDL cholesterol.

In another embodiment, the present invention provides a method of treating a lipid disorder selected from the group consisting of dyslipidemia, hyperlipidemia, hypertriglyceridemia, hypercholesterolemia, low HDL and high LDL in a mammalian patient in need of such treatment comprising administering to said patient a Benzo-Fused Oxazepine Compound in an amount that is effective to treat said lipid disorder.

In yet another embodiment, the present invention provides a method of treating atherosclerosis in a mammalian patient in need of such treatment comprising administering to said patient a Benzo-Fused Oxazepine Compound in an amount effective to treat atherosclerosis.

In yet another embodiment, the present invention provides a method of treating cancer in a mammalian patient in need of such treatment comprising administering to said patient a Benzo-Fused Oxazepine Compound in an amount effective to treat cancer.

In still another embodiment, the invention provides a method for treating a skin disorder, including but not limited to eczema, acne, psoriasis, keloid scar formation or prevention, oily skin, shiny or greasy-looking skin, seborrheic dermatitis, disorders related to production or secretions from mucous membranes, such as monounsaturated fatty acids, wax esters, and the like in a mammalian patient in need of such treatment comprising administering to the patient a Benzo-Fused Oxazepine Compound in an amount that is effective to treat such a skin disorder. In particular instances the skin disorder being treated is acne.

In addition, the present invention provides a method of treating a cosmetic condition such as greasy or oily-looking hair, comprising administering to the patient a Benzo-Fused Oxazepine Compound in an amount that is effective to treat such cosmetic condition.

In another embodiment, the present invention provides a method of treating a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) a skin disorder, (24) greasy or oily-looking hair, (25) metabolic syndrome, and (26) other conditions and disorders where insulin resistance is a component, in a mammalian patient in need of such treatment comprising administering to the patient a Benzo-Fused Oxazepine Compound in an amount that is effective to treat said condition.

In still another embodiment, the present invention provides a method of delaying the onset of a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) a skin disorder, (24) greasy or oily-looking hair, (25) metabolic syndrome, and (26) other conditions and disorders where insulin resistance is a component, in a mammalian patient in need of such treatment comprising administering to the patient a Benzo-Fused Oxazepine Compound in an amount that is effective to delay the onset of said condition.

In another embodiment, the present invention provides a method of reducing the risk of developing a condition selected from the group consisting of (1) hyperglycemia, (2) low glucose tolerance, (3) insulin resistance, (4) obesity, (5) lipid disorders, (6) dyslipidemia, (7) hyperlipidemia, (8) hypertriglyceridemia, (9) hypercholesterolemia, (10) low HDL levels, (11) high LDL levels, (12) atherosclerosis and its sequelae, (13) vascular restenosis, (14) pancreatitis, (15) abdominal obesity, (16) neurodegenerative disease, (17) retinopathy, (18) nephropathy, (19) neuropathy, (20) fatty liver disease, (21) polycystic ovary syndrome, (22) sleep-disordered breathing, (23) a skin disorder, (24) greasy or oily-looking hair, (25) metabolic syndrome, and (26) other conditions and disorders where insulin resistance is a component, in a mammalian patient in need of such treatment comprising administering to the patient a Benzo-Fused Oxazepine Compound in an amount that is effective to reduce the risk of developing said condition.

In another aspect, the invention provides a method for treating a condition where increasing lean body mass or lean muscle mass is desired, such as is desirable in enhancing performance through muscle building, comprising administering to a patient in need of such treatment an amount of a Benzo-Fused Oxazepine Compound effective treating such condition. Myopathies and lipid myopathies such as carnitine palmitoyltransferase deficiency (CPT I or CPT II) are also included as such conditions. Such treatments are useful in humans and in animal husbandry, including for administering to bovine, porcine or avian domestic animals or any other animal to reduce triglyceride production and/or provide leaner meat products and/or healthier animals.

### Combination Therapy

In another embodiment, the present methods for treating or preventing a viral infection or a virus-related disorder can further comprise the administration of one or more additional therapeutic agents which are not Benzo-Fused Oxazepine Compounds.

The compounds of the present invention may be used in combination with one or more other agents in the treatment, prevention, suppression or amelioration of diseases or conditions for which the Benzo-Fused Oxazepine Compounds or the other agents may have utility, where the combination of the drugs together are safer or more effective than either agent alone. Such other agent(s) may be administered, by a route and in an amount commonly used therefore, contemporaneously or sequentially with a Benzo-Fused Oxazepine Compound. When a Benzo-Fused Oxazepine Compound is used contemporaneously with one or more other therapeutic agents, a pharmaceutical composition in unit dosage form containing such other agents and the Benzo-Fused Oxazepine Compound is preferred. However, the combination therapy may also include therapies in which the Benzo-Fused Oxazepine Compound and one or more other agents are administered on different overlapping schedules. In some embodiments, when used in combination with one or more other therapeutic agents, the Benzo-Fused Oxazepine Compounds and the other therapeutic agents may be used in lower doses than when each is used singly.

Accordingly, as discussed further below, the pharmaceutical compositions of the present invention include those that contain one or more other therapeutic agents, in addition to a Benzo-Fused Oxazepine Compound.

Examples of other therapeutic agents that may be administered in combination with a Benzo-Fused Oxazepine Compound, and either administered separately or in the same pharmaceutical composition, include, but are not limited to:

(a) dipeptidyl peptidase IV (DPP-IV) inhibitors;

(b) insulin sensitizers including (i) PPAR-gamma-agonists, such as the glitazones (e.g., troglitazone, pioglitazone, englitazone, MCC-555, rosiglitazone, balaglitazone, and the like) and other PPAR ligands, including PPAR alpha/gamma dual agonists, such as KRP-297, muraglitazar, naveglitazar, Galida, TAK-559, PPAR gamma agonists, such as fenofibric acid derivatives (gemfibrozil, clofibrate, fenofibrate and bezafibrate), and selective PPAR-gamma-modulators (SPPAR-gammaM's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) .alpha-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPAR-alpha agonists such as fenofibric acid derivatives (gemfibrozil, clofibrate, fenofibrate and bezafibrate), (v) PPAR-alpha/.gamma dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPAR-delta agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, .beta₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11-beta-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476.

(t) antibiotic agents, such as tetracycline and clindamycin;

(u) retinoids, such as etretinate, tretinoin, and aliretinoin; and

(v) estrogen and progesterone.

Dipeptidyl peptidase-IV inhibitors that can be combined with the Benzo-Fused Oxazepine Compounds include those disclosed in U.S. Pat. No. 6,699,871; WO 02/076450; WO 03/004498; WO 03/004496; EP 1 258 476; WO 02/083128; WO 02/062764; WO 03/000250; WO 03/002530; WO 03/002531; WO 03/002553; WO 03/002593; WO 03/000180; WO 03/082817; WO 03/000181; WO 04/007468; WO 04/032836; WO 04/037169; and WO 04/043940. Specific DPP-IV inhibitor compounds include isoleucine thiazolidide (P32/98); NVP-DPP-728; LAF 237; P93/01; and saxagliptin (BMS 477118).

Antiobesity compounds that can be combined with the Benzo-Fused Oxazepine Compounds include fenfluramine, dexfenfluramine, phentermine, sibutramine, orlistat, neuropeptide Y₁ or Y₅ antagonists, cannabinoid CBI receptor antagonists or inverse agonists, melanocortin receptor agonists, in particular, melanocortin-4 receptor agonists, ghrelin antagonists, bombesin receptor agonists, and melanin-concentrating hormone (MCH) receptor antagonists.

Neuropeptide Y5 antagonists that can be combined with the Benzo-Fused Oxazepine Compounds include those disclosed in U.S. Pat. No. 6,335,345 and WO 01/14376; and specific compounds identified as GW 59884A; GW 569180A; LY366377; and CGP-71683A.

Cannabinoid CB1 receptor antagonists that can be combined with Benzo-Fused Oxazepine Compounds include those disclosed in PCT Publication WO 03/007887; U.S. Pat. No. 5,624,941, such as rimonabant; PCT Publication WO 02/076949, such as SLV-319; U.S. Pat. No. 6,028,084; PCT Publication WO 98/41519; PCT Publication WO 00/10968; PCT Publication WO 99/02499; U.S. Pat. No. 5,532,237; U.S. Pat. No. 5,292,736; PCT Publication WO 03/086288; POT Publication WO 03/087037; POT Publication WO 04/048317; POT Publication WO 03/007887; PCT Publication WO 03/063781; PCT Publication WO 03/075660; POT Publication WO 03/077847; PCT Publication WO 03/082190; POT Publication WO 03/082191; PCT Publication WO 03/087037; PCT Publication WO 03/086288; PCT Publication WO 04/012671; PCT Publication WO 04/029204; PCT Publication WO 04/040040; PCT Publication WO 01/64632; PCT Publication WO 01/64633; and POT Publication WO 01/64634.

Melanocortin-4 receptor (MC4R) agonists useful in combination with the Benzo-Fused Oxazepine Compounds include, but are not limited to, those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entireties; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entireties; and in WO 99/64002, WO 00/74679, WO 02/15909, WO 01/70708, WO 01/70337, WO 01/91752, WO 02/068387, WO 02/068388, WO 02/067869, WO 03/007949, WO 2004/024720, WO 2004/089307, WO 2004/078716, WO 2004/078717, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/009850, WO 03/013571, WO 03/031410, WO 03/053927, WO 03/061660, WO 03/066597, WO 03/094918, WO 03/099818, WO 04/037797, WO 04/048345, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/093234, WO 03/095474, and WO 03/104761.

One particular aspect of combination therapy relates to a method of treating a condition selected from the group consisting of hypercholesterolemia, atherosclerosis, low HDL levels, high LDL levels, hyperlipidemia, hypertriglyceridemia, and dyslipidemia, in a mammalian patient in need of such treatment comprising administering to the patient a therapeutically effective amount of a Benzo-Fused Oxazepine Compound and an HMG-CoA reductase inhibitor.

More particularly, this aspect of combination therapy concerns a method of treating a condition selected from the group consisting of hypercholesterolemia, atherosclerosis, low HDL levels, high LDL levels, hyperlipidemia, hypertriglyceridemia and dyslipidemia in a mammalian patient in need of such treatment wherein the HMG-CoA reductase inhibitor is a statin selected from the group consisting of lovastatin, simvastatin, pravastatin, cerivastatin, fluvastatin, atorvastatin, and rosuvastatin.

Another aspect of the invention relates to a method of reducing the risk of developing a condition selected from the group consisting of hypercholesterolemia, atherosclerosis, low HDL levels, high LDL levels, hyperlipidemia, hypertriglyceridemia and dyslipidemia, and the sequelae of such conditions is disclosed comprising administering to a mammalian patient in need of such treatment a therapeutically effective amount of a Benzo-Fused Oxazepine Compound and an HMG-CoA reductase inhibitor.

In another aspect, the invention provides a method for delaying the onset or reducing the risk of developing atherosclerosis in a human patient in need of such treatment comprising administering to said patient an effective amount of a Benzo-Fused Oxazepine Compound and an HMG-CoA reductase inhibitor. In particular embodiments, the HMG-CoA reductase inhibitor is a statin selected from the group consisting of: lovastatin, simvastatin, pravastatin, cerivastatin, fluvastatin, atorvastatin, and rosuvastatin.

In another aspect, the invention provides a method for delaying the onset or reducing the risk of developing atherosclerosis in a human patient in need of such treatment, comprising administering to said patient a Benzo-Fused Oxazepine Compound, a statin-type HMG-CoA reductase inhibitor, and further administering a cholesterol absorption inhibitor. In particular embodiments, the cholesterol absorption inhibitor is ezetimibe.

In another aspect, the invention provides a method for treating acne in a human patient in need of such treatment, comprising administering to said patient a Benzo-Fused Oxazepine Compound and an antibiotic, such as tetracycline or clindamycin. The antibiotic is useful for eradicating the effect of the microorganism, Propionibacterium acnes, which contributes to developing acne.

In another aspect, the invention provides a method for treating acne in a human patient in need of such treatment, comprising administering to said patient a Benzo-Fused Oxazepine Compound and a retinoid, such as etretinate, tretinoin, and aliretinoin.

In another aspect, the invention provides a method for treating acne in a human patient in need of such treatment, comprising administering to said patient a Benzo-Fused Oxazepine Compound and estrogen or progesterone.

When administering a second therapeutic agent in combination with a Benzo-Fused Oxazepine Compound, the weight ratio of the Benzo-Fused Oxazepine Compound to the second agent may be varied and will depend upon the effective dose of each agent. Generally, an effective dose of each will be used. Thus, for example, when a Benzo-Fused Oxazepine Compound is combined with another agent, the weight ratio of the compound of the present invention to the other agent will generally range from about 1000:1 to about 1:1000, preferably about 200:1 to about 1:200. Combinations of a Benzo-Fused Oxazepine Compound and other therapeutic agents will generally also be within the aforementioned range, but in each case, an effective dose of each active ingredient should be used.

In such combinations the Benzo-Fused Oxazepine Compound and other therapeutic agents may be administered separately or in conjunction. In addition, the administration of one therapeutic agent may be prior to, concurrent to, or subsequent to the administration of other agent(s).

### Compositions and Administration

This invention is also directed to pharmaceutical compositions which comprise at least one Benzo-Fused Oxazepine Compound, or a pharmaceutically acceptable salt of said compound and at least one pharmaceutically acceptable carrier.

When administered to a patient, the Benzo-Fused Oxazepine Compounds can be administered as a component of a composition that comprises a pharmaceutically acceptable carrier or vehicle. The present invention provides pharmaceutical compositions comprising an effective amount of at least one Benzo-Fused Oxazepine Compound and a pharmaceutically acceptable carrier. In the pharmaceutical compositions and methods of the present invention, the active ingredients will typically be administered in admixture with suitable carrier materials suitably selected with respect to the intended form of administration, i.e., oral tablets, capsules (either solid-filled, semi-solid filled or liquid filled), powders for constitution, oral gels, elixirs, dispersible granules, syrups, suspensions, and the like, and consistent with conventional pharmaceutical practices. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa. For example, for oral administration in the form of tablets or capsules, the active drug component may be combined with any oral non-toxic pharmaceutically acceptable inert carrier, such as lactose, starch, sucrose, cellulose, magnesium stearate, dicalcium phosphate, calcium sulfate, talc, mannitol, ethyl alcohol (liquid forms) and the like. Solid form preparations include powders, tablets, dispersible granules, capsules, cachets and suppositories. Powders and tablets may be comprised of from about 0.5 to about 95 percent inventive composition. Tablets, powders, cachets and capsules can be used as solid dosage forms suitable for oral administration.

Moreover, when desired or needed, suitable binders, lubricants, disintegrating agents and coloring agents may also be incorporated in the mixture. Suitable binders include starch, gelatin, natural sugars, corn sweeteners, natural and synthetic gums such as acacia, sodium alginate, carboxymethylcellulose, polyethylene glycol and waxes. Among the lubricants there may be mentioned for use in these dosage forms, boric acid, sodium benzoate, sodium acetate, sodium chloride, and the like. Disintegrants include starch, methylcellulose, guar gum, and the like. Sweetening and flavoring agents and preservatives may also be included where appropriate.

Liquid form preparations include solutions, suspensions and emulsions and may include water or water-propylene glycol solutions for parenteral injection.

Liquid form preparations may also include solutions for intranasal administration.

Liquid form preparations may include compositions suitable for topical applications, such as are used for dermatological applications. For instance, in one embodiment, the Benzo-Fused Oxazepine Compound is present in a vehicle containing propylene glycol:transcutanol:ethanol (20:20:60, v/v/v) and propylene glycol:ethanol (30:70, v/v). In some embodiments, the Benzo-Fused Oxazepine Compound may be present in the topical composition at concentrations of between about 1.5% to about 2.0% (w/v).

Aerosol preparations suitable for inhalation may include solutions and solids in powder form, which may be in combination with a pharmaceutically acceptable carrier, such as an inert compressed gas.

Also included are solid form preparations which are intended to be converted, shortly before use, to liquid form preparations for either oral or parenteral administration. Such liquid forms include solutions, suspensions and emulsions.

For preparing suppositories, a low melting wax such as a mixture of fatty acid glycerides or cocoa butter is first melted, and the active ingredient is dispersed homogeneously therein as by stirring. The molten homogeneous mixture is then poured into convenient sized molds, allowed to cool and thereby solidify.

The Benzo-Fused Oxazepine Compounds of the present invention may also be delivered transdermally. The transdermal compositions can take the form of creams, lotions, aerosols, foams and/or emulsions and can be included in a transdermal patch of the matrix or reservoir type as are conventional in the art for this purpose.

Additionally, the compositions of the present invention may be formulated in sustained release form to provide the rate controlled release of any one or more of the components or active ingredients to optimize therapeutic effects, i.e., anti-cancer activity and the like. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

In one embodiment, the Benzo-Fused Oxazepine Compound is administered orally.

In another embodiment, the Benzo-Fused Oxazepine Compound is administered intravenously.

In still another embodiment, the Benzo-Fused Oxazepine Compound is administered sublingually.

In another embodiment, the Benzo-Fused Oxazepine Compound is administered topically, for example, for use in treating a skin disorder of the type described above. Typically, in such embodiments, the Benzo-Fused Oxazepine Compound is a component of topical composition which can take the form of solutions, salves, creams, ointments, in liposomal formulations, sprays, gels, lotions, aerosols, foams, emulsions, or any other formulation routinely used in dermatology. Such topical compositions can be administered using a patch, e.g., of the matrix type, or a roller stick, as are conventional in the art for this purpose.

In one embodiment, a pharmaceutical preparation comprising at least one Benzo-Fused Oxazepine Compound is in unit dosage form. In such form, the preparation is subdivided into unit doses containing effective amounts of the active components.

Compositions can be prepared according to conventional mixing, granulating or coating methods, respectively, and the present compositions can contain, in one embodiment, from about 0.1% to about 99% of the Benzo-Fused Oxazepine Compound(s) by weight or volume. In various embodiments, the present compositions can contain, in one embodiment, from about 1% to about 70% or from about 5% to about 60% of the Benzo-Fused Oxazepine Compound(s) by weight or volume.

The quantity of Benzo-Fused Oxazepine Compound in a unit dose of preparation may be varied or adjusted from about 1 mg to about 2500 mg. In various embodiments, the quantity is from about 10 mg to about 1000 mg, 1 mg to about 500 mg, 1 mg to about 100 mg, and 1 mg to about 50 mg.

For convenience, the total daily dosage may be divided and administered in portions during the day if desired. In one embodiment, the daily dosage is administered in one portion. In another embodiment, the total daily dosage is administered in two divided doses over a 24 hour period. In another embodiment, the total daily dosage is administered in three divided doses over a 24 hour period. In still another embodiment, the total daily dosage is administered in four divided doses over a 24 hour period.

For administration to human patients, the amount and frequency of administration of the Benzo-Fused Oxazepine Compound will be regulated according to the judgment of the attending clinician considering such factors as age, condition and size of the patient as well as severity of the symptoms being treated. Generally, a total daily dosage of the Benzo-Fused Oxazepine Compound is in the range of from about 0.1 to about 3000 mg per day, although variations will necessarily occur depending on the target of therapy, the patient and the route of administration. In one embodiment, the dosage is from about 1 to about 300 mg/day, administered in a single dose or in 2-4 divided doses. In another embodiment, the dosage is from about 10 to about 3000 mg/day, administered in a single dose or in 2-4 divided doses. In another embodiment, the dosage is from about 100 to about 3000 mg/day, administered in a single dose or in 2-4 divided doses. In still another embodiment, the dosage is from about 500 to about 3000 mg/day, administered in a single dose or in 2-4 divided doses.

For dermatological administration, such as for the treatment of a skin disorder, the dose of the Benzo-Fused Oxazepine Compound will vary, but typically the compound will be present in a pharmaceutically acceptable composition in an amount of from about 0.01 to 50 w/w %, and more typically from about 0.1 to 10 w/w %. In some embodiments, the formulation may be applied to the affected area from 1 to 4 times daily.

The compositions of the invention can further comprise one or more additional therapeutic agents, selected from those listed above herein. Accordingly, in one embodiment, the present invention provides compositions comprising: (i) at least one Benzo-Fused Oxazepine Compound or a pharmaceutically acceptable salt thereof; (ii) one or more additional therapeutic agents that are not a Benzo-Fused Oxazepine Compound; and (iii) a pharmaceutically acceptable carrier, wherein the amounts in the composition are together effective to treat disease or disorder associated with aberrant SCD activity.

In certain embodiments the compositions of the invention, a pharmaceutical composition is disclosed which comprise:

(1) a Benzo-Fused Oxazepine Compound; (3) a pharmaceutically acceptable carrier; and (3) a compound selected from the group consisting of:

(a) dipeptidyl peptidase IV (DPP-IV) inhibitors;

(b) insulin sensitizers including (i) PPAR gamma agonists, such as the glitazones (e.g., troglitazone, pioglitazone, englitazone. MCC-555, rosiglitazone, balaglitazone, and the like) and other PPAR ligands, including PPAR alpha/gamma dual agonists, such as KRP-297, muraglitazar, naveglitazar, Galida, TAK-559, PPAR alpha agonists, such as fenofibric acid derivatives (gemfibrozil, clofibrate, fenofibrate and bezafibrate), and selective PPAR gamma modulators (SPPAR gamma M's), such as disclosed in WO 02/060388, WO 02/08188, WO 2004/019869, WO 2004/020409, WO 2004/020408, and WO 2004/066963; (ii) biguanides such as metformin and phenformin, and (iii) protein tyrosine phosphatase-1B (PTP-1B) inhibitors;

(c) insulin or insulin mimetics;

(d) sulfonylureas and other insulin secretagogues, such as tolbutamide, glyburide, glipizide, glimepiride, and meglitinides, such as nateglinide and repaglinide;

(e) alpha-glucosidase inhibitors (such as acarbose and miglitol);

(f) glucagon receptor antagonists, such as those disclosed in WO 98/04528, WO 99/01423, WO 00/39088, and WO 00/69810;

(g) GLP-1, GLP-1 analogues or mimetics, and GLP-1 receptor agonists, such as exendin-4 (exenatide), liraglutide (N,N-2211), CJC-1131, LY-307161, and those disclosed in WO 00/42026 and WO 00/59887;

(h) GIP and GIP mimetics, such as those disclosed in WO 00/58360, and GIP receptor agonists;

(i) PACAP, PACAP mimetics, and PACAP receptor agonists such as those disclosed in WO 01/23420;

(j) cholesterol lowering agents such as (i) HMG-CoA reductase inhibitors (lovastatin, simvastatin, pravastatin, cerivastatin, fluvastatin, atorvastatin, itavastatin, and rosuvastatin, and other statins), (ii) sequestrants (cholestyramine, colestipol, and dialkylaminoalkyl derivatives of a cross-linked dextran), (iii) nicotinyl alcohol, nicotinic acid or a salt thereof, (iv) PPAR alpha agonists such as fenofibric acid derivatives (gemfibrozil, clofibrate, fenofibrate and bezafibrate), (v) PPAR alpha/gamma dual agonists, such as naveglitazar and muraglitazar, (vi) inhibitors of cholesterol absorption, such as beta-sitosterol and ezetimibe, (vii) acyl CoA:cholesterol acyltransferase inhibitors, such as avasimibe, and (viii) antioxidants, such as probucol;

(k) PPAR delta agonists, such as those disclosed in WO 97/28149;

(l) antiobesity compounds, such as fenfluramine, dexfenfluramine, phentermine, sibutramine, orlistat, neuropeptide Y₁ or Y₅ antagonists, CB1 receptor inverse agonists and antagonists, beta₃ adrenergic receptor agonists, melanocortin-receptor agonists, in particular melanocortin-4 receptor agonists, ghrelin antagonists, bombesin receptor agonists (such as bombesin receptor subtype-3 agonists), and melanin-concentrating hormone (MCH) receptor antagonists;

(m) ileal bile acid transporter inhibitors;

(n) agents intended for use in inflammatory conditions such as aspirin, non-steroidal anti-inflammatory drugs (NSAIDs), glucocorticoids, azulfidine, and selective cyclooxygenase-2 (COX-2) inhibitors;

(o) antihypertensive agents, such as ACE inhibitors (enalapril, lisinopril, captopril, quinapril, tandolapril), A-II receptor blockers (losartan, candesartan, irbesartan, valsartan, telmisartan, and eprosartan), beta blockers and calcium channel blockers;

(p) glucokinase activators (GKAs), such as those disclosed in WO 03/015774; WO 04/076420; and WO 04/081001;

(q) inhibitors of 11 beta-hydroxysteroid dehydrogenase type 1, such as those disclosed in U.S. Pat. No. 6,730,690; WO 03/104207; and WO 04/058741;

(r) inhibitors of cholesteryl ester transfer protein (CETP), such as torcetrapib;

(s) inhibitors of fructose 1,6-bisphosphatase, such as those disclosed in U.S. Pat. Nos. 6,054,587; 6,110,903; 6,284,748; 6,399,782; and 6,489,476;

(t) antibiotic agents, such as tetracycline and clindamycin;

(u) retinoids, such as etretinate, tretinoin, and aliretinoin; and

(v) estrogen and progesterone.

### Kits

Another aspect of this invention is a kit comprising a therapeutically effective amount of at least one Benzo-Fused Oxazepine Compound, or a pharmaceutically acceptable salt of said compound and a pharmaceutically acceptable carrier, vehicle or diluent.

Yet another aspect of this invention is a kit comprising an amount of at least one Benzo-Fused Oxazepine Compound, or a pharmaceutically acceptable salt of said compound and an amount of at least one additional therapeutic agent listed above, wherein the amounts of the two or more active ingredients result in a desired therapeutic effect. In one embodiment, the at least one Benzo-Fused Oxazepine Compound and the at least one additional therapeutic agent are provided in the same container. In one embodiment, the at least one Benzo-Fused Oxazepine Compound and the at least one additional therapeutic agent are provided in separate containers.

Another aspect of this invention is a kit containing the at least one Benzo-Fused Oxazepine Compound (and any additional therapeutic agents) packaged for retail distribution (i.e., an article of manufacture or a kit). Such articles will be labeled and packaged in a manner to instruct the patient how to use the product. Such instructions will include the condition to be treated, duration of treatment, dosing schedule, etc.

The present invention is not to be limited by the specific embodiments disclosed in the examples that are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

A number of references have been cited herein, the entire disclosures of which are incorporated herein by reference.

What is claimed is:

1. A compound having the formula (I): 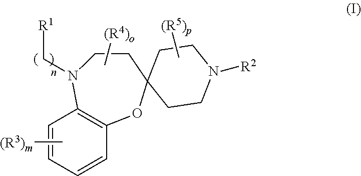
or a pharmaceutically acceptable salt thereof, wherein
R¹ is aryl or heteroaryl, wherein said aryl or heteroaryl of R¹ is unsubstituted or substituted with one to four moieties, wherein said moieties are the same or different, and wherein said moieties are selected from the group consisting of alkyl, alkoxy, haloalkyl, halo, —CN, —C(O)—R⁶, —OCF₃, and R⁷;
R² is heteroaryl, wherein said heteroaryl is unsubstituted or substituted with one to three moieties, which moieties are the same or different, and wherein said moieties are selected from Z, wherein Z is Z¹ or Z²;
Z¹ is alkyl, alkoxy, halo, haloalkyl, —CN, —C(O)—OH, —C(O)—O-alkyl, —C(O)—O-cycloalkyl, —C(O)—N(R¹²)₂, —OCF₃, aryl, heteroaryl, aryl substituted with alkyl, and heteroaryl substituted by alkyl;
Z² is 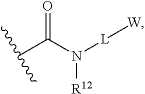
wherein L is a direct bond such that W is bonded directly to the illustrated N atom of —N(R¹²)—, or L is —(CH₂)ₓ—, —CH₂—C(H)(OH)—, or —CH₂—C(H)(OH)—OH₂—;
W is —C(O)OR¹³, —C(O)NR¹², —S(O)alkyl, —S(O)₂alkyl, —CF₃, —C(H)(OH)—CH₂OH, —CH₂OH, —C(H)(CH₃)OH, cycloalkyl, aryl, heteroaryl, heterocyclyl, wherein said cycloalkyl, aryl, heteroaryl, or heterocyclyl of W is unsubstituted or substituted with one to three moieties, wherein said moieties are selected from the group consisting of alkyl, hydroxyl, alkoxy, halo, —CF₃, —OCF₃, or —CN;
with the proviso that when W is —C(O)OR¹³, —C(O)NR¹², —S(O)alkyl, —S(O)₂alkyl, —CF₃, —C(H)(OH)—CH₂OH, or —CH₂OH or, then L must be —(CH₂)ₓ—, —CH₂—C(H)(OH)—, or —CH₂—C(H)(OH)—CH₂—;
each R³ is independently alkyl, alkoxy, or halo;
each R⁴ is independently alkyl or halo;
each R⁵ is independently alkyl or halo;
R⁶ is —OH, —O-alkyl, —O-cycloalkyl, —N(R¹²)₂,
R⁷ is —O-aryl, —O-heteroaryl, —N(R¹²)-aryl, or —N(R¹²)-heteroaryl,
each R¹² is independently H, alkyl, alkoxyethyl, alkoxypropyl, dialkylaminoethyl, dialkylaminopropyl, or wherein two R¹² are geminally substituted on a N atom, the two R¹² together with the N atom on which they are substituted form a 4- to 8-membered heterocyclyl;
R¹³ is H, alkyl, or cycloalkyl;
m is 0, 1, 2, 3, or 4;
n is 1, 2, or 3;
o is 0 or 1;
p is 0, 1, 2, 3, or 4; and
x is 1, 2, 3, 4, or 5.

2. The compound of claim 1, or a pharmaceutically acceptable salt thereof, wherein said heteroaryl of R² comprises at least one nitrogen atom as a ring member, and wherein said heteroaryl is substituted with one to three moieties, which moieties are the same or different, and wherein said moieties are selected from the group consisting of alkyl, alkoxy, halo, haloalkyl, —CN, —C(O)—OH, —C(O)—O-alkyl, —C(O)-β-cycloalkyl, —C(O)—N(R¹²)₂, —OCF₃, aryl, heteroaryl, aryl substituted with alkyl, heteroaryl substituted by alkyl, and Z².

3. The compound of claim 2, or a pharmaceutically acceptable salt thereof, wherein said heteroaryl of R² is substituted with one —CN, —C(O)—N(R¹²)₂, —C(O)—OH, heteroaryl substituted with alkyl, or Z².

4. The compound of claim 3 or a pharmaceutically acceptable salt thereof, wherein said substituted heteroaryl of R² is thiazole, pyridine, or pyridazine.

5. The compound of claim 1 or a pharmaceutically acceptable salt thereof, wherein n is 1.

6. The compound of claim 1 or a pharmaceutically acceptable salt thereof, wherein R¹ is phenyl substituted with one to four moieties, wherein said moieties are the same or different, and wherein said moieties are selected from the group consisting of alkyl, alkoxy, haloalkyl, halo, —CN, —C(O)—R⁶, —OCF₃, and R⁷.

7. The compound of claim 1 or a pharmaceutically acceptable salt thereof, wherein R³ is halo and m is 1.

8. The compound of claim 1 or a pharmaceutically acceptable salt thereof, wherein
R¹ is phenyl substituted with one alkyl, —CF₃, alkoxy, or —O-phenyl;
R² is heteroaryl substituted with one to three moieties, which moieties are the same or different, and wherein said moieties are selected from the group consisting of alkyl, alkoxy, halo, haloalkyl, —CN, —C(O)—OH, —C(O)—O-alkyl, —C(O)—O-cycloalkyl, —C(O)—N(R¹²)₂, —OCF₃, aryl, heteroaryl, aryl substituted with alkyl, heteroaryl substituted by alkyl, and Z²;
R³ is halo;
m is 0 or 1;
n is 1;
o is 0; and
p is 0.

9. The compound of claim 8 or a pharmaceutically acceptable salt thereof, wherein
said heteroaryl of R² is substituted with one Z², wherein R¹² of said Z² is H,
L is —(CH)ₓ— and
W is —C(O)OR¹³, —C(O)N(R¹²)₂, —C(H)(OH)—CH₂OH, —CH₂OH, —C(H)(CH₃)OH, cycloalkyl, or heterocyclyl.

10. The compound of claim 9 or a pharmaceutically acceptable salt thereof, wherein said heteroaryl of R² is thiazole, pyridine, or pyridazine.

11. The compound of claim 1 or a pharmaceutically acceptable salt thereof, wherein the compound has the Formula (Ib): 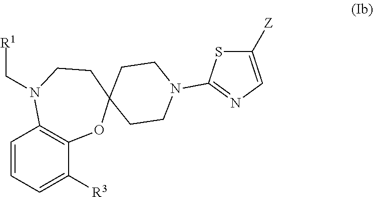
and wherein the compound is selected from the compounds in the table below, wherein R¹, R³, and Z in Formula (Ib) are as described in the table below:

| Compound | R¹ | Z | R³ |
|---|---|---|---|
| 50 | 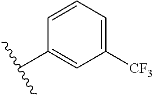 | 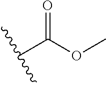 | H |
| 51 | 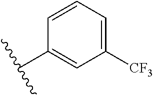 | 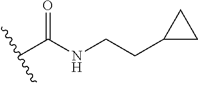 | H |
| 52 | 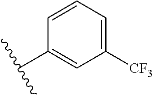 | 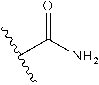 | H |
| 53 | 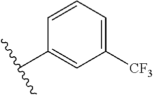 | 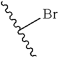 | H |
| 54 | 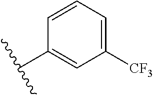 | 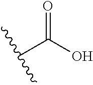 | H |
| 55 | 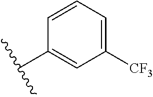 | 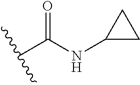 | H |
| 56 | 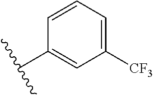 | 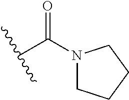 | H |
| 57 | 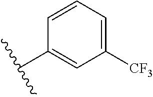 | 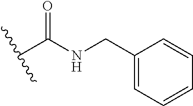 | H |
| 58 | 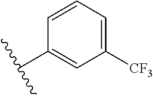 | 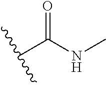 | H |
| 59 | 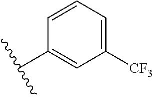 | 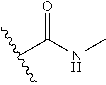 | H |
| 60 | 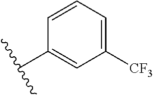 | 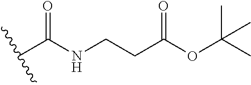 | H |
| 61 | 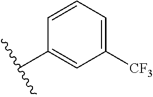 | 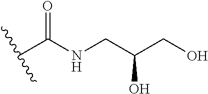 | H |
| 62 | 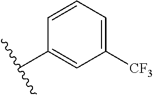 | 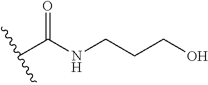 | H |
| 63 | 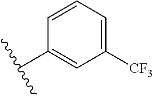 | 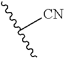 | H |
| 64 | 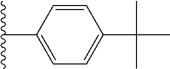 | 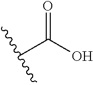 | H |
| 65 | 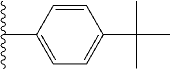 | 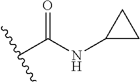 | H |
| 66 | 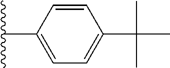 | 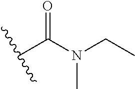 | H |
| 67 | 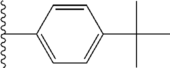 | 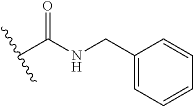 | H |
| 68 | 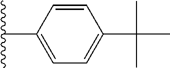 | 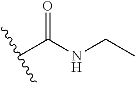 | H |
| 69 | 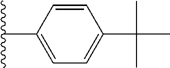 | 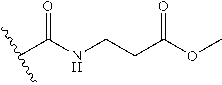 | H |
| 70 | 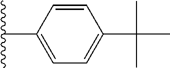 | 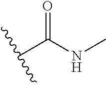 | H |
| 72 | 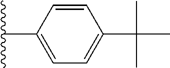 | 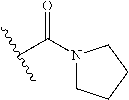 | H |
| 73 | 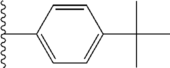 | 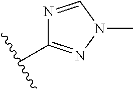 | H |
| 74 | 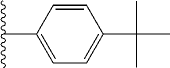 | 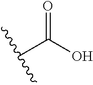 | F |
| 75 | 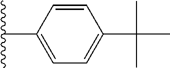 | 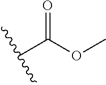 | F |
| 76 | 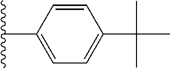 | 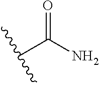 | F |
| 77 | 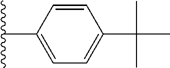 | 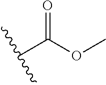 | F |
| 78 | 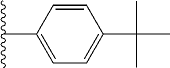 | 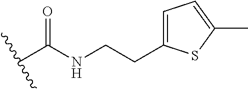 | F |
| 79 | 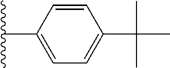 | 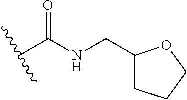 | F |
| 80 | 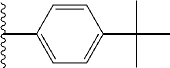 | 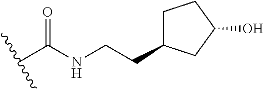 | F |
| 81 | 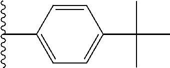 | 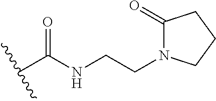 | F |
| 83 | 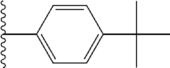 | 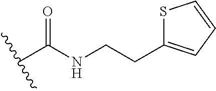 | F |
| 84 | 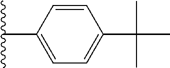 | 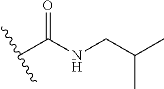 | F |
| 85 | 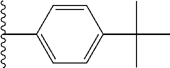 | 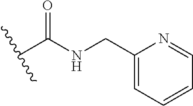 | F |
| 86 | 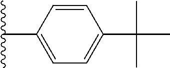 | 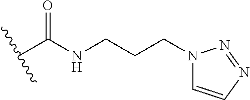 | F |
| 87 | 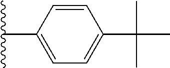 | 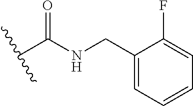 | F |
| 88 | 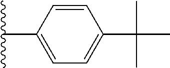 | 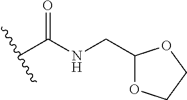 | F |
| 89 | 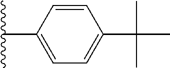 | 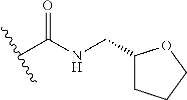 | F |
| 90 | 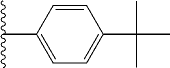 | 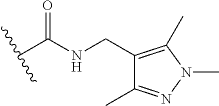 | F |
| 91 | 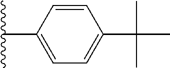 | 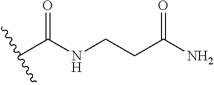 | F |
| 92 | 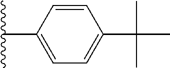 | 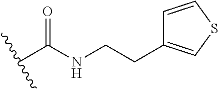 | F |
| 93 | 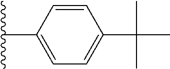 | 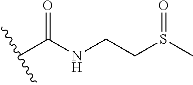 | F |
| 94 | 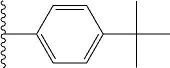 | 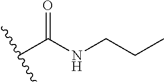 | F |
| 95 | 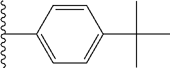 | 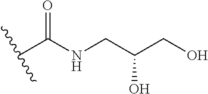 | F |
| 96 | 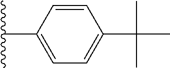 | 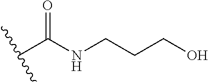 | F |
| 97 | 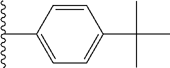 | 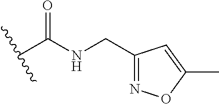 | F |
| 98 | 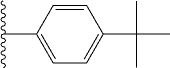 | 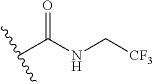 | F |
| 99 | 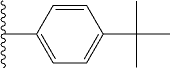 | 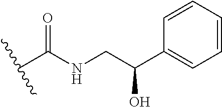 | F |
| 101 | 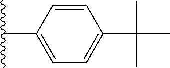 | 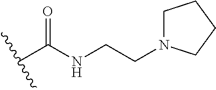 | F |
| 102 | 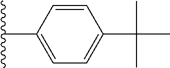 | 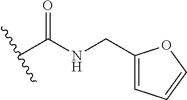 | F |
| 103 | 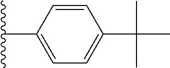 | 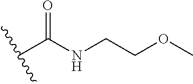 | F |
| 104 | 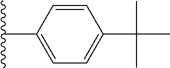 | 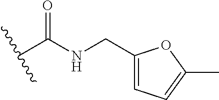 | F |
| 107 | 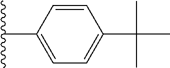 | 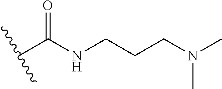 | F |
| 108 | 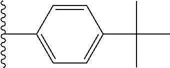 | 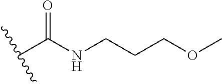 | F |
| 109 | 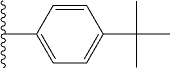 | 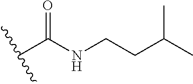 | F |
| 110 | 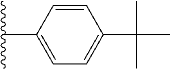 | 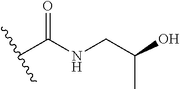 | F |
| 112 | 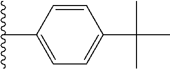 | 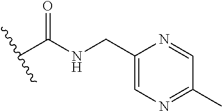 | F |
| 113 | 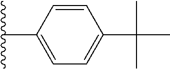 | 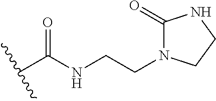 | F |
| 114 | 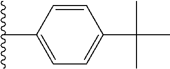 | 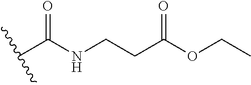 | F |
| 115 | 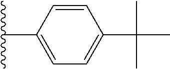 | 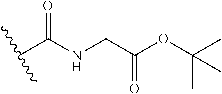 | F |
| 116 | 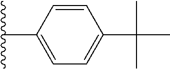 | 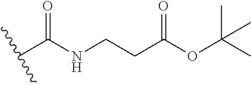 | F |
| 118 | 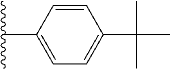 | 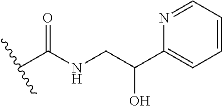 | F |
| 120 | 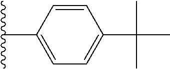 | 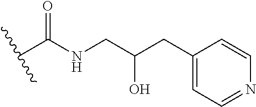 | F |
| 123 | 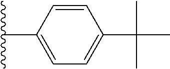 | 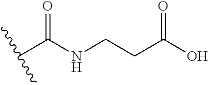 | F |
| 124 | 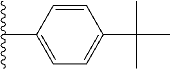 | 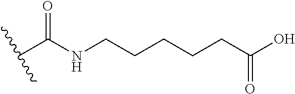 | F |
| 125 | 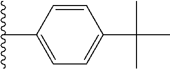 | 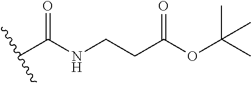 | H |
| 126 | 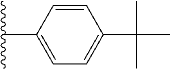 | 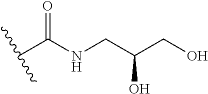 | H |
| 127 | 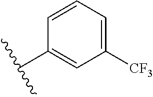 | 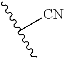 | H |
| 128 | 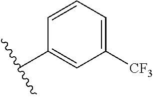 | 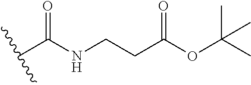 | H |
| 129 | 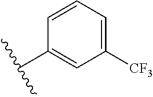 | 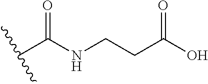 | H |
| 130 | 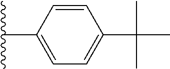 | 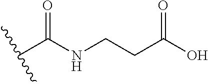 | H |
| 131 | 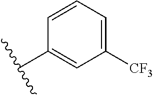 | 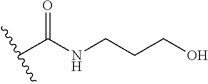 | F |
| 132 | 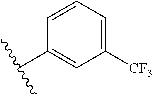 | 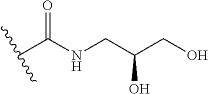 | F |
| 133 | 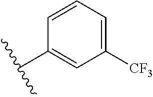 | 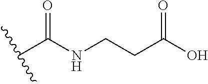 | F |
| 134 | 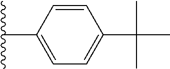 | 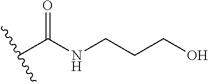 | H |
| 135 | 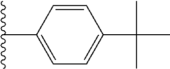 | 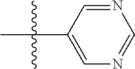 | H |
| 136 | 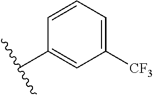 | 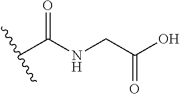 | H |
| 137 | 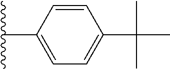 | 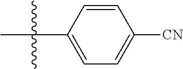 | H |
| 138 | 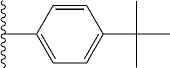 | 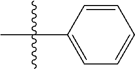 | H. |

12. The compound of claim 1 or a pharmaceutically acceptable salt thereof, wherein the compound has the Formula (Ic) 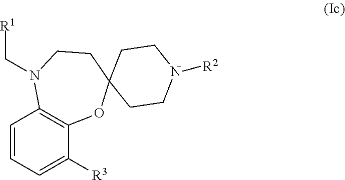
and wherein the compound is selected from the compounds in the table below, wherein R¹, R², and R³ in Formula (Ic) are as described in the table below:

| Compound | R¹ | R² | R³ |
|---|---|---|---|
| 139 | 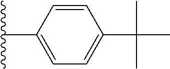 | 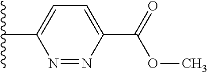 | H |
| 140 | 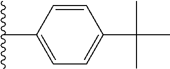 | 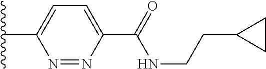 | H |
| 141 | 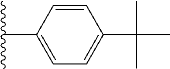 | 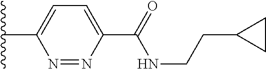 | H |
| 142 | 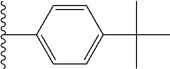 | 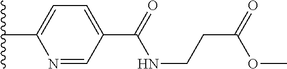 | F |
| 143 | 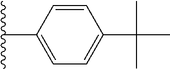 | 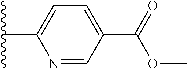 | F |
| 144 | 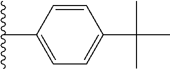 | 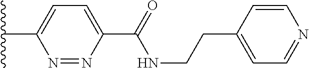 | F |
| 145 | 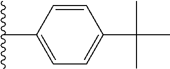 | 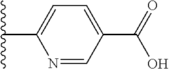 | F |
| 146 | 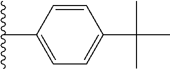 | 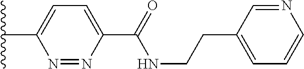 | F |
| 147 | 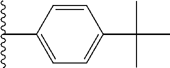 | 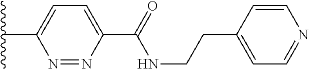 | F |
| 148 | 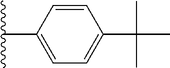 | 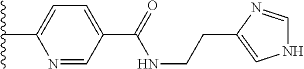 | F |
| 149 | 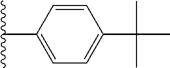 | 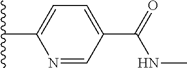 | F |
| 150 | 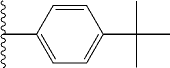 | 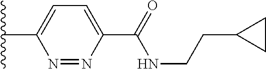 | F |
| 151 | 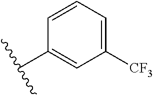 | 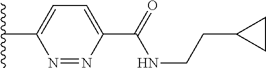 | H |
| 152 | 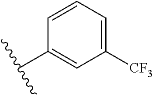 | 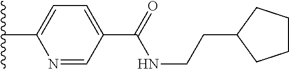 | H |
| 153 | 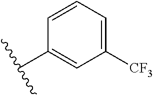 | 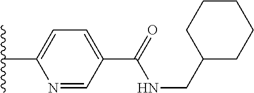 | H |
| 154 | 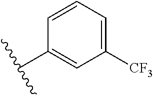 | 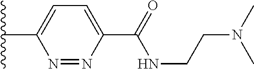 | H |
| 155 | 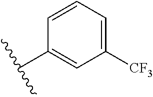 | 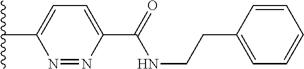 | H |
| 156 | 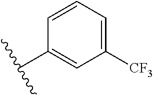 | 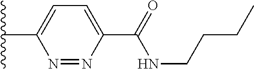 | H |
| 157 | 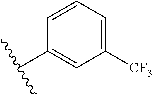 | 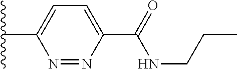 | H |
| 158 | 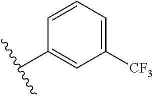 | 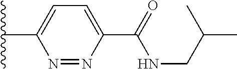 | H |
| 159 | 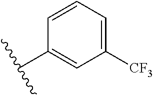 | 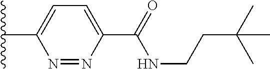 | H |
| 160 | 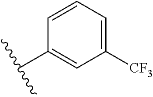 | 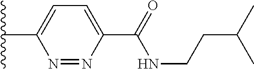 | H |
| 161 | 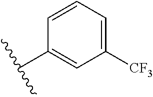 | 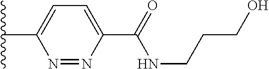 | H |
| 162 | 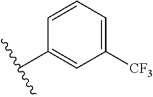 | 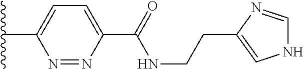 | H |
| 163 | 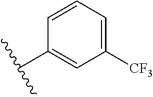 | 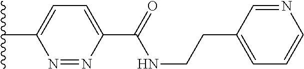 | H |
| 164 | 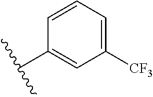 | 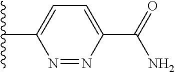 | H |
| 165 | 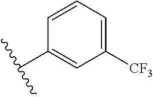 | 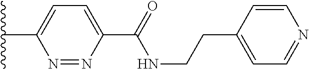 | H |
| 166 | 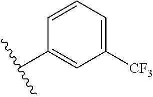 | 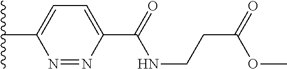 | H |
| 167 | 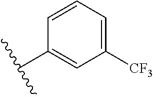 | 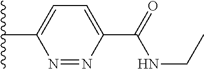 | H |
| 168 | 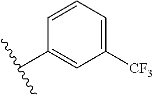 | 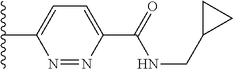 | H |
| 169 | 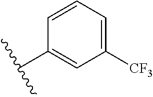 | 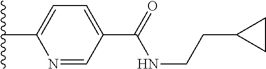 | H |
| 170 | 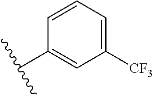 | 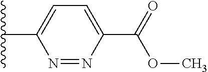 | H |
| 171 | 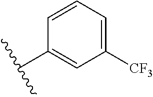 | 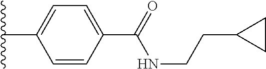 | H |
| 172 | 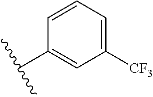 | 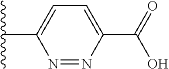 | H |
| 173 | 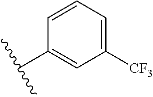 | 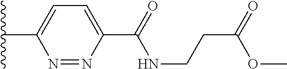 | F |
| 174 | 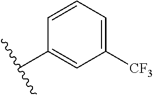 | 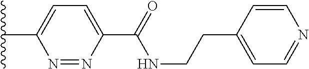 | F |
| 175 | 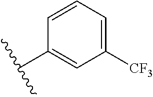 | 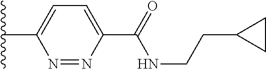 | F |
| 176 | 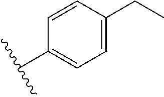 | 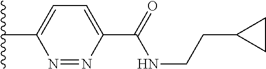 | H |
| 177 | 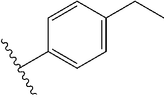 | 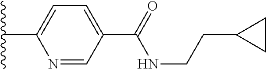 | H |
| 178 | 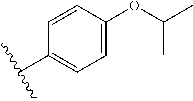 | 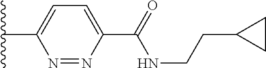 | H |
| 179 | 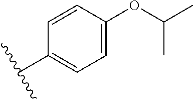 | 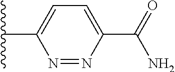 | H |
| 180 | 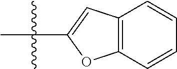 | 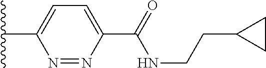 | H |
| 181 | 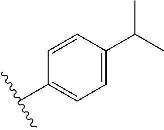 | 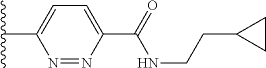 | H |
| 182 | 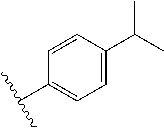 | 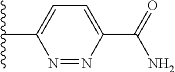 | H |
| 183 | 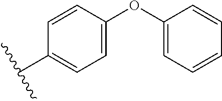 | 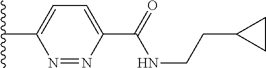 | H |
| 184 | 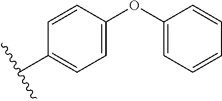 | 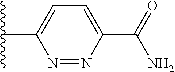 | H. |

13. A pharmaceutical composition comprising the compound of claim 1 or a pharmaceutically acceptable salt thereof, and a pharmaceutically acceptable carrier.

14. A method of treating a disorder selected from non-insulin dependent (Type 2) diabetes, insulin resistance, hyperglycemia, a lipid disorder, obesity, fatty liver disease, or a skin disorder comprising administering a compound of claim 1, or a pharmaceutically acceptable salt thereof, to a patient in need of such treatment.

15. The method of claim 14, wherein said disorder is a lipid disorder, and wherein said lipid disorder is dyslipidemia, hyperlipidemia, atherosclerosis, hypercholesterolemia, low LDL, or high LDL.

16. The method of claim 14, wherein said disorder is a skin disorder.

17. A compound having the formula (I): 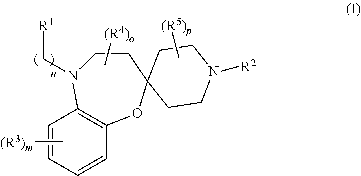
or a pharmaceutically acceptable salt thereof, wherein
R¹ is aryl or heteroaryl, wherein said aryl or heteroaryl of R¹ is unsubstituted or substituted with one to four moieties, wherein said moieties are the same or different, and wherein said moieties are selected from the group consisting of alkyl, alkoxy, haloalkyl, halo, —CN, —C(O)—R⁶, —OCF₃, and R⁷;
R² is —C(O)—Y, wherein Y is alkyl or cycloalkyl, 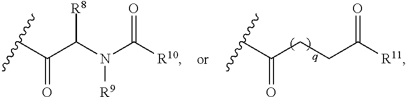
or R² is aryl or heteroaryl, wherein said aryl or heteroaryl is unsubstituted or substituted with one to three moieties, which moieties are the same or different, and wherein said moieties are selected from Z, wherein Z is Z¹ or Z²;
Z¹ is alkyl, alkoxy, halo, haloalkyl, —CN, —C(O)—OH, —C(O)—O-cycloalkyl, —C(O)—N(R¹²)₂, —OCF₃, aryl, heteroaryl, aryl substituted with alkyl, and heteroaryl substituted by alkyl;
Z² is 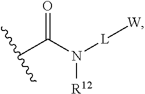
wherein L is a direct bond such that W is bonded directly to the illustrated N atom of —N(R¹²)—, or L is —(CH₂)—, —CH₂—C(H)(OH)—, or —CH₂—C(H)(OH)—CH₂—;
W is —C(O)OR¹³, —C(O)NR¹², —S(O)alkyl, —S(O)₂alkyl, —CF₃, —C(H)(OH)—CH₂OH, —CH₂OH, —C(H)(CH₃)OH, cycloalkyl, aryl, heteroaryl, heterocyclyl, wherein said cycloalkyl, aryl, heteroaryl, or heterocyclyl of W is unsubstituted or substituted with one to three moieties, wherein said moieties are selected from the group consisting of alkyl, hydroxyl, alkoxy, halo, —CF₃, —OCF₃, or
—CN;
with the proviso that when W is —C(O)OR¹³, —C(O)NR¹², —S(O)alkyl, —S(O)₂alkyl, —CF₃, —C(H)(OH)—CH₂OH, or —CH₂OH or, then L must be —(CH₂)—, —CH₂—C(H)(OH)—, or —CH₂—C(H)(OH)—CH₂—;
each R³ is independently alkyl, alkoxy, or halo;
each R⁴ is independently alkyl or halo;
each R⁵ is independently alkyl or halo;
R⁶ is —OH, —O-alkyl, —O-cycloalkyl, —N(R¹²)₂,
R⁷ is —O-aryl, —O-heteroaryl, —N(R¹²)-aryl, or —N(R¹²)-heteroaryl,
R⁸ is H, alkyl, or hydroxyalkyl;
R⁹ is H or alkyl;
R¹⁰ is alkyl, cycloalkyl, aryl, heteroaryl, or heterocyclyl;
R¹¹ is OH, alkyl, or cycloalkyl;
each R¹² is independently H, alkyl, alkoxyethyl, alkoxypropyl, dialkylaminoethyl, dialkylaminopropyl, or wherein two R¹² are geminally substituted on a N atom, the two R¹² together with the N atom on which they are substituted form a 4- to 8-membered heterocyclyl;
R¹³ is H, alkyl, or cycloalkyl;
m is 0, 1, 2, 3, or 4;
n is 1, 2, or 3;
is 0 or 1;
p is 0, 1, 2, 3, or 4;
q is 1 or 2; and
x is 1, 2, 3, 4, or 5;
with the proviso that the compound is other than
N-(2-oxo-2-(5-(4-phenoxybenzyl)-4,5-dihydro-3H-spiro[benzo[b][1,4]oxazepine-2,4′-piperidine]-1′-yl)ethyl)acetamide;
N-(2-(5-(4-isopropoxybenzyl)-4,5-dihydro-3H-spiro[benzo[b][1,4]oxazepine-2,4′-piperidine]-1′-yl)-2-oxoethyl)acetamide;
N-(2-(5-(biphenyl-4-ylmethyl)-4,5-dihydro-3H-spiro[benzo[b][1,4]oxazepine-2,4′-piperidine]-1′-yl)-2-oxoethyl)acetamide;
N-(2-(5-(4-tert-butylbenzyl)-4,5-dihydro-3H-spiro[benzo[b][1,4]oxazepine-2,4′-piperidine]-1′-yl)-2-oxoethyl)acetamide;
N-(2-(5-(4-isopropylbenzyl)-4,5-dihydro-3H-spiro[benzo[b][1,4]oxazepine-2,4′-piperidine]-1′-yl)-2-oxoethyl)acetamide;
4-(5-(4-isopropylbenzyl)-4,5-dihydro-3H-spiro[benzo[b][1,4]oxazepine-2,4′-piperidine]-1′-yl)-4-oxobutanamide;
N-(2-(5-(4-(4-fluorobenzyloxy)benzyl)-4,5-dihydro-3H-spiro[benzo[b][1,4]oxazepine-2,4′-piperidine]-1′-yl)-2-oxoethyl)acetamide;
4-(5-(4-tert-butylbenzyl)-4,5-dihydro-3H-spiro[benzo[b][1,4]oxazepine-2,4′-piperidine]-1′-yl)-4-oxobutanamide;
4-(5-(biphenyl-4-ylmethyl)-4,5-dihydro-3H-spiro[benzo[b][1,4]oxazepine-2,4′-piperidine]-1′-yl)-4-oxobutanamide;
5-(4-ethylbenzyl)-N-(4-(methoxymethyl)phenyl)-4,5-dihydro-3H-spiro[benzo[b][1,4]oxazepine-2,4′-piperidine]-1′-carboxamide;
2-(benzo[c][1,2,5]thiadiazol-4-yl)-1-(5-(4-isopropylbenzyl)-4,5-dihydro-3H-spiro[benzo[b][1,4]oxazepine-2,4′-piperidine]-1′-yl)ethanone;
N-(2-(5-(benzofuran-2-ylmethyl)-4,5-dihydro-3H-spiro[benzo[b][1,4]oxazepine-2,4′-piperidine]-1′-yl)-2-oxoethyl)furan-2-carboxamide;
N-(2-oxo-2-(5-(3-(trifluoromethyl)benzyl)-4,5-dihydro-3H-spiro[benzo[b][1,4]oxazepine-2,4′-piperidine]-1′-yl)ethyl)furan-2-carboxamide;
4-(5-(4-isopropoxybenzyl)-4,5-dihydro-3H-spiro[benzo[b][1,4]oxazepine-2,4′-piperidine]-1-1′-yl)-4-oxobutanamide;
N-(2-(5-(2-ethylbenzyl)-4,5-dihydro-3H-spiro[benzo[b][1,4]oxazepine-2,4′-piperidine]-1′-yl)-2-oxoethyl)acetamide;
N-(benzo[c][1,2,5]thiadiazol-4-yl)-5-(biphenyl-4-ylmethyl)-4,5-dihydro-3H-spiro[benzo[b][1,4]oxazepine-2,4′-piperidine]-1′-carboxamide;
3-(5-(4-tert-butylbenzyl)-4,5-dihydro-3H-spiro[benzo[b][1,4]oxazepine-2,4′-piperidine]-1-yl)-3-oxopropanenitrile;
N-(benzo[c][1,2,5]thiadiazol-4-yl)-5-(4-tert-butylbenzyl)-4,5-dihydro-3H-spiro[benzo[b][1,4]oxazepine-2,4′-piperidine]-1′-carboxamide;
methyl 4-((1′-(2-acetamidoacetyl)-3H-spiro[benzo[b][1,4]oxazepine-2,4′-piperidine]-5(4H)-yl)methyl)benzoate;
N-(2-(5-(2,3-dimethylbenzyl)-4,5-dihydro-3H-spiro[benzo[b][1,4]oxazepine-2,4′-piperidine]-1′-yl)-2-oxoethyl)acetamide;
4-oxo-4-(5-(4-phenoxybenzyl)-4,5-dihydro-3H-spiro[benzo[b][1,4]oxazepine-2,4′-piperidine]-1-yl)butanamide;
N-(2-(5-((5-(3-chlorophenyl)furan-2-yl)methyl)-4,5-dihydro-3H-spiro[benzo[b][1,4]oxazepine-2,4′-piperidine]-1′-yl)-2-oxoethyl)furan-2-carboxamide;
N-(2-oxo-2-(5-(4-(pyridin-2-yl)benzyl)-4,5-dihydro-3H-spiro[benzo[b][1,4]oxazepine-2,4′-piperidine]-1′-yl)ethyl)acetamide;
N-(2-(5-(3-fluoro-2-methylbenzyl)-4,5-dihydro-3H-spiro[benzo[b][1,4]oxazepine-2,4′-piperidine]-1′-yl)-2-oxoethyl)acetamide;
N-(benzo[c][1,2,5]thiadiazol-4-yl)-5-(4-phenoxybenzyl)-4,5-dihydro-3H-spiro[benzo[b][1,4]oxazepine-2,4′-piperidine]-1′-carboxamide;
N-(2-(5-(2,5-difluorobenzyl)-4,5-dihydro-3H-spiro[benzo[b][1,4]oxazepine-2,4′-piperidine]-1′-yl)-2-oxoethyl)acetamide;
N-(3,5-difluorophenyl)-5-(4-isopropylbenzyl)-4,5-dihydro-3H-spiro[benzo[b][1,4]oxazepine-2,4′-piperidine]-1′-carboxamide;
3-oxo-3-(5-(3-(trifluoromethyl)benzyl)-4,5-dihydro-3H-spiro[benzo[b][1,4]oxazepine-2,4′-piperidine]-1′-yl)propanenitrile;
N-(2-(5-(4-fluoro-3-methoxybenzyl)-4,5-dihydro-3H-spiro[benzo[b][1,4]oxazepine-2,4′-piperidine]-1′-yl)-2-oxoethyl)acetamide;
5-(2-oxo-2-(5-(3-(trifluoromethyl)benzyl)-4,5-dihydro-3H-spiro[benzo[b][1,4]oxazepine-2,4′-piperidine]-1′-yl)ethyl)imidazolidine-2,4-dione;
N-(2-(5-(4-ethylbenzyl)-4,5-dihydro-3H-spiro[benzo[b][1,4]oxazepine-2,4′-piperidine]-1-yl)-2-oxoethyl)benzamide;
N-(2-oxo-2-(5-(3-phenylpropyl)-4,5-dihydro-3H-spiro[benzo[b][1,4]oxazepine-2,4′-piperidine]-1′-yl)ethyl)acetamide;
N-(2-(5-(3-methoxybenzyl)-4,5-dihydro-3H-spiro[benzo[b][1,4]oxazepine-2,4′-piperidine]-1′-yl)-2-oxoethyl)acetamide;
N-(2-(5-(2-ethoxybenzyl)-4,5-dihydro-3H-spiro[benzo[b][1,4]oxazepine-2,4′-piperidine]-1′-yl)-2-oxoethyl)acetamide;
4-oxo-4-(5-(3-(trifluoromethyl)benzyl)-4,5-dihydro-3H-spiro[benzo[b][1,4]oxazepine-2,4′-piperidine]-1′-yl)butanamide;
N-(2-oxo-2-(5-(3-(trifluoromethyl)benzyl)-4,5-dihydro-3H-spiro[benzo[b][1,4]oxazepine-2,4′-piperidine]-1′-yl)ethyl)benzamide;
4-(5-((5-(3-chlorophenyl)furan-2-yl)methyl)-4,5-dihydro-3H-spiro[benzo[b][1,4]oxazepine-2,4′-piperidine]-1′-yl)-4-oxobutanamide;
4-(5-(2-ethylbenzyl)-4,5-dihydro-3H-spiro[benzo[b][1,4]oxazepine-2,4′-piperidine]-1′-yl)-4-oxobutanamide;
N-(2-oxo-2-(5-(thiophen-2-ylmethyl)-4,5-dihydro-3H-spiro[benzo[b][1,4]oxazepine-2,4′-piperidine]-1′-yl)ethyl)acetamide;
3-(5-(biphenyl-4-ylmethyl)-4,5-dihydro-3H-spiro[benzo[b][1,4]oxazepine-2,4′-piperidine]-1′-yl)-3-oxopropanenitrile;
3-(5-(4-isopropylbenzyl)-4,5-dihydro-3H-spiro[benzo[b][1,4]oxazepine-2,4′-piperidine]-1′-yl)-3-oxopropanenitrile;
5-(4-ethylbenzyl)-N-(4-fluorophenyl)-4,5-dihydro-3H-spiro[benzo[b][1,4]oxazepine-2,4′-piperidine]-1′-carboxamide;
(2S,3S)-methyl 2-(5-(4-ethylbenzyl)-4,5-dihydro-3H-spiro[benzo[b][1,4]oxazepine-2,4′-piperidine]-1′-ylcarboxamido)-3-methylpentanoate;
N-(2-(5-(4-ethylbenzyl)-4,5-dihydro-3H-spiro[benzo[b][1,4]oxazepine-2,4′-piperidine]-1′-yl)-2-oxoethyl)acetamide; and
N-(2-oxo-2-(5-(3-(trifluoromethyl)benzyl)-4,5-dihydro-3H-spiro[benzo[b][1,4]oxazepine-2,4′-piperidine]-1′-yl)ethyl)acetamide.

18. A compound having the Formula (Ia) 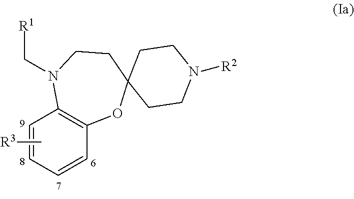
or a pharmaceutically acceptable salt thereof; wherein the compound is selected from the compounds in the table below, wherein R¹, R², and R³ in Formula (Ia) are as described in the table below:

| Compound | R¹ | R² | R³ |
|---|---|---|---|
| 4 | 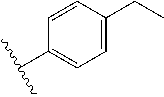 | 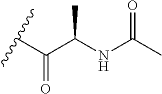 | H |
| 5 | 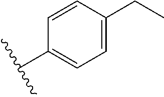 | 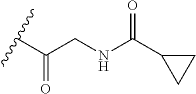 | H |
| 6 | 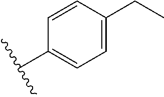 | 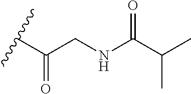 | H |
| 7 | 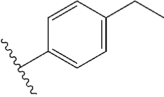 | 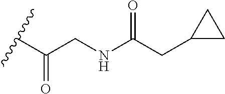 | H |
| 8 | 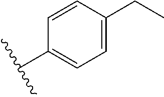 | 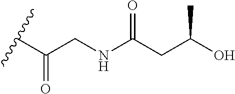 | H |
| 11 | 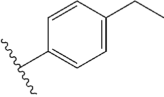 | 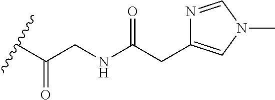 | H |
| 12 | 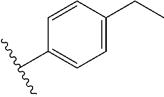 | 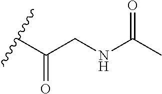 | 9-F |
| 13 | 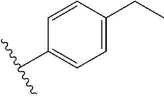 | 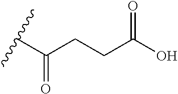 | H |
| 14 | 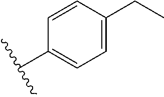 | 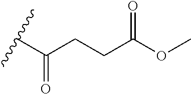 | H |
| 15 | 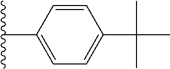 | 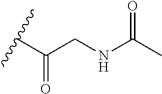 | 6-Cl |
| 16 | 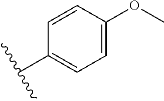 | 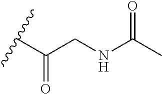 | H |
| 21 | 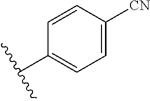 | 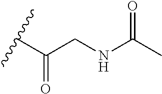 | H |
| 22 | 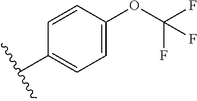 | 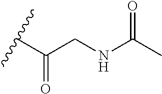 | H |
| 24 | 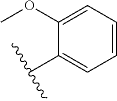 | 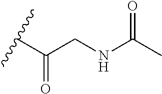 | H |
| 25 | 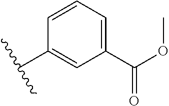 | 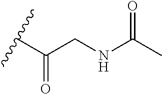 | H |
| 26 | 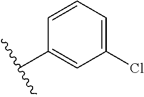 | 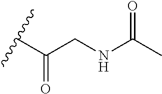 | H |
| 27 | 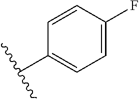 | 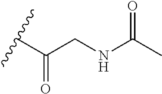 | H |
| 28 | 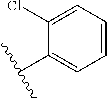 | 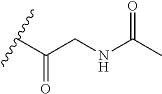 | H |
| 29 | 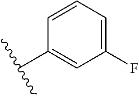 | 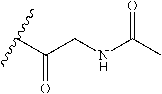 | H |
| 30 | 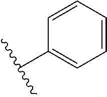 | 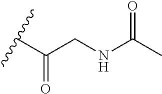 | H |
| 31 | 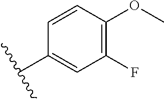 | 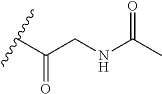 | H |
| 32 | 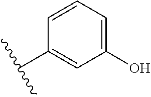 | 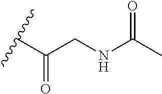 | H |
| 33 | 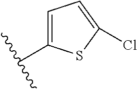 | 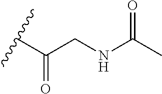 | H |
| 34 | 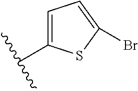 | 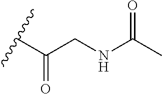 | H |
| 35 | 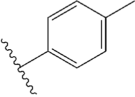 | 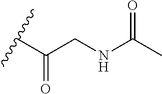 | H |
| 36 | 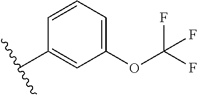 | 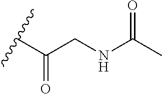 | H |
| 37 | 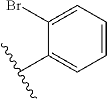 | 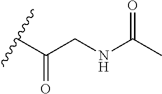 | H |
| 38 | 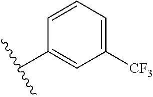 | 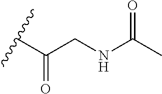 | 7-F |
| 39 | 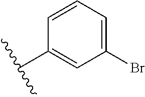 | 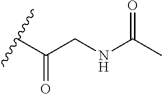 | H |
| 40 | 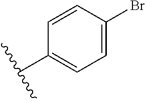 | 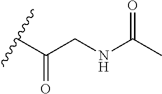 | H |
| 41 | 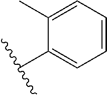 | 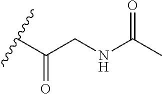 | H |
| 42 | 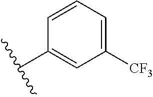 | 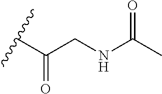 | 9-Cl |
| 43 | 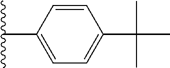 | 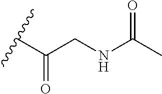 | 9-Cl |
| 44 | 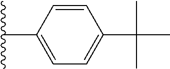 | 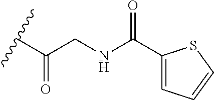 | H |
| 45 | 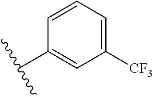 | 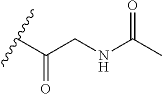 | 6-F |
| 46 | 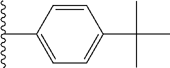 | 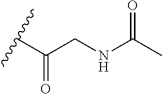 | 6-F |
| 47 | 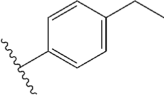 | 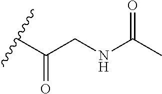 | 6-F |
| 48 | 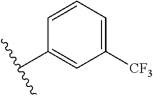 | 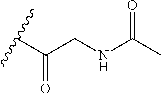 | 9-F |
| 49 | 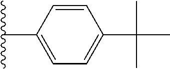 | 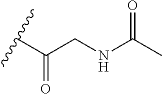 | 9-F. |
